(19) United States
(12) Patent Application Publication
(10) Pub. No.: US 2023/0189819 A1
(43) Pub. Date: Jun. 22, 2023

(54) COMPOSITIONS AND METHODS FOR FUNGAL INHIBITION USING MINICELL-BASED RNAi

(71) Applicant: AgroSpheres, Inc., Charlottesville, VA (US)

(72) Inventors: Payam POURTAHERI, Charlottesville, VA (US); Ameer Hamza SHAKEEL, Charlottesville, VA (US); Lisa CHEN, Charlottesville, VA (US); Joseph FRANK, Charlottesville, VA (US); Sepehr ZOMORODI, Charlottesville, VA (US)

(21) Appl. No.: 17/926,069

(22) PCT Filed: May 19, 2021
(86) PCT No.: PCT/US2021/033208

§ 371 Date: Nov. 17, 2022

Related U.S. Application Data
(60) Provisional application No. 63/027,044, filed on May 19, 2020.
(60) Provisional application No. 63/039,506, filed on Jun. 16, 2020.

(51) Int. Cl. A01N 63/60; C12N 15/113; C12N 15/88; A01N 25/28; A01P 3/00

(52) U.S. Cl. CPC A01N 63/60; A01N 25/28; A01P 3/00; C12N 15/88; C12N 15/1137; C12N 2310/14; C12N 2310/51

(57) ABSTRACT

It is herewith provided a biocontrol composition comprising an intact minicell comprising a nucleic acid that targets a transcript encoding a polypeptide and a method for controlling pests and pathogens with novel systems and compositions to deliver RNA molecules to a subject.

### CROSS REFERENCE TO RELATED APPLICATIONS

This application claims the benefit of priority to U.S. Provisional Application No. 63/027,044 filed on May 19, 2020 and U.S. Provisional Application No. 63/039,506 filed on Jun. 16, 2020, each of which is hereby incorporated by reference in its entirety.

### DESCRIPTION OF THE TEXT FILE SUBMITTED ELECTRONICALLY

The contents of the text file submitted electronically herewith are incorporated herein by reference in their entirety: A computer readable format copy of the Sequence Listing filename: AGRO_006_01WO_SeqList_ST25.txt, date recorded, May 18, 2021, file size ≈ 105 kilobytes.

### FIELD

The present disclosure generally relates to compositions and methods for controlling pests and pathogens, such as fungal and fungal-like pathogens. Also provided are compositions and methods for down-regulating targeted genes using RNA molecules. The disclosure further relates to the biocontrol of pests and pathogens using novel systems, compositions, and methods for the biocontrol of pests and pathogens through minicell technology.

### BACKGROUND

Each year, gray mold disease caused by the fungal pathogen Botrytis cinerea, leads to $10 to $100 billion of global agricultural losses (Petrasch et al 2019). Predominant control strategies for gray mold disease heavily rely on chemical fungicidal products (Rupp et al 2017; Fernández-Ortuño et al 2015). The frequent and prophylactic use of these chemical fungicides has led to ecotoxicological harm, human health risk and resistance development in fungal pathogens (Amiri et al 2013; Weber et al 2011; Panebianco et al 2015; Konstantinou et al 2015).

Due to the prolonged usage of broad-spectrum fungicides, various fungal pathogens have developed resistance to a majority of the fungicides on the market. The need for alternative pesticides, especially those with novel modes of actions, is high. These repercussions evidence the urgent need for alternative biocontrols that are effective and environmentally sustainable.

### SUMMARY OF THE DISCLOSURE

The following embodiments and aspects thereof are described in conjunction with systems, tools and methods which are meant to be exemplary, not limiting in scope. In various embodiments, one or more of the above-described problems have been reduced or eliminated, while other embodiments are directed to other improvements.

The present disclosure provides agricultural compositions or formulations comprising minicells for encapsulation and delivery of biocontrol compounds to a desired subject such as a pest or a pathogen, or a plant suffering therefrom. These compositions can be developed into standalone biocontrol products for application directly to a pest or a pathogen, or a plant suffering therefrom, or can be added into other agricultural products for enhanced plant survival, growth, or yield with a synergistic effect. Also, the present disclosure provide methods of controlling pathogens or preventing plants from harmful damages caused by the pathogens with biocontrol compositions or formulations taught herein.

The present disclosure is drawn to biocontrol compositions and methods for controlling pests and pathogens, such as fungal and fungal-like pathogens. In some embodiments, the biocontrol compositions works for down-regulating targeted genes using RNA interference, which are inhibitory RNA molecules protected and carried by a minicell system.

The present disclosure provides a biocontrol composition comprising: a minicell comprising at least one polynucleotide encoding an inhibitory RNA molecule that are directed to at least one target sequence in a pathogen. In some embodiments, the inhibitory RNA molecule causes downregulation of said at least one target sequence in the pathogen. In some embodiments, the pathogen or a disease caused by the pathogen is suppressed upon application of the composition compared to a control pathogen lacking the application of the composition.

In some embodiments, the pathogen is suppressed, inhibited, limited, controlled or killed upon application of the composition compared to a control pathogen lacking the application of the composition. In some embodiments, a disease or condition caused by the pathogen is prevented, treated or cured upon application of the composition compared to a control pathogen lacking the application of the composition.

In some embodiments, the minicell is derived from a bacterial cell. In some embodiments, the minicell is derived from a bacterial cell having a mislocalized cell division. In further embodiments, the minicell is derived from a bacterial cell having a mislocalized cell division with deficient RNaseIII activity. In further embodiments, the minicell is derived from a bacterial cell having a mislocalized cell division with deficient protease activity. In further embodiments, the minicell is derived from a bacterial cell having a mislocalized cell division with both RNaseIII and protease activities deficient. In some embodiments, the inhibitory RNA molecule is an antisense RNA, a single-stranded RNA (ssRNA), a double-stranded RNA (dsRNA), a hairpin RNA (hpRNA), a short-hairpin RNA (shRNA), a small-interfering RNA (siRNA), a microRNA (miRNA), or combination thereof. In some embodiments, the inhibitory RNA molecule is exogenously prepared and encapsulated by the minicell. In other embodiments, the inhibitory RNA molecule is transcribed from at least one heterologous expression cassette comprising a promoter operably linked to the at least one polynucleotide within the minicell.

In some embodiments, the target sequence is selected from genes encoding Argonaute family proteins, chitin synthases, dicer-like proteins, proteins encoded by resistance genes, ABC family proteins, proteins encoded by meiotic silencing genes, sex-induced silencing proteins, proteins composed of the RISC Complex, and combination thereof. In further embodiments, the target sequence is a Chitin Synthase 3a gene, a Chitin Synthase 3b gene, a Dicer-like 1 gene, a Dicer-like 2 gene or combination thereof.

In some embodiments, the inhibitory RNA molecule targets a nucleic acid sequence encoding an amino acid sequence at least 90% identical to SEQ ID NO: 18 or 20. In some embodiments, the inhibitory RNA molecule targets a nucleic acid sequence encoding an amino acid sequence at least 90% identical to SEQ ID NO:22 or 24.

In some embodiments, the pathogen is selected from the group consisting of Albugo, Alternaria; Aphanomyces; Aspergillus; Ascochyta; Blumeria; Botrytis; Cercospora; Colletotrichum; Diplodia; Erysiphe; Fusarium; Gaeumannomyces; Helminthosporium; Leptosphaeria: Macrophomina; Magnaporthe; Melampsora; Mycospharella; Nectria; Penecillium; Peronospora; Phakopsora; Phialophora, Phoma; Phymatotrichum; Phytophthora; Plasmopara; Plasmodiophora; Podosphaera; Puccinia; Pythium; Pyrenophora; Pyricularia; Rhizoctonia; Scerotium; Sclerotinia; Septoria; Stemphylium; Thielaviopsis; Uncinula; Uromyces; Ustilago; Venturia; and Verticillium. In further embodiments, the pathogen is Alternaria alternata, Botrytis cinerea, Fusarium solani, Peronospora berteroae, Phakospora pachyrhizi, Penicillium expansum, Phialophora gregata, Phytophthora sojae, Sclerotinia sclerotiorum or Verticillium dahliae. In further embodiments, the pathogen is Botrytis cinerea.

In some embodiments, said minicell is applied with at least one agriculturally suitable carrier. In some embodiments, the carrier is a solid, liquid, emulsion or powder form. In further embodiments, the carrier increases stability, wettability, or dispersability. In some embodiments, the composition is applied to a subject by spraying, injecting, soaking, brushing, dressing, dripping, or coating in a solution.

In some embodiments, the subject is a plant selected from the group consisting of: corn, wheat, rice, barley, oat, rye, sorghum, millet, sugar cane, strawberry, blueberry, raspberry, blackberry, apple, grape, pear, peach, melon, cucumber, pumpkin, squash, soybean, sugar beet, spinach, swiss chard, potato, eggplant, tomato, sunflower, safflower, gladiolus, cotton, canola, alfalfa, cannabis, Brassica, peanut, tobacco, banana, duckweed, pineapple, date, onion, cashew, pistachio, citrus, rose, almond, coffee, bean, legume, watermelon, squash, cabbage, turnip, mustard, cacti, pecan, flax, sweet potato, coconut, avocado, cantaloupe, vegetables, and herbs. In further embodiments, the subject is a strawberry plant.

The present disclosure provides a biocontrol composition comprising: a minicell comprising at least one polynucleotide encoding an inhibitory RNA molecule that targets a nucleic acid sequence encoding an amino acid sequence at least 90% identical to SEQ ID NO:18, 20, 22 or 24, wherein the sequence is present in a pathogen. In some embodiments, the pathogen or a disease caused by the pathogen is suppressed upon application of the composition compared to a control pathogen lacking the application of the composition.

The present disclosure provides a method for controlling a pathogen, comprising the steps of: introducing into a minicell at least one polynucleotide encoding an inhibitory RNA molecule that are directed to at least one target sequence in a pathogen, and applying the minicell to a subject. In some embodiments, the inhibitory RNA molecule causes downregulation of said at least one target sequence in the pathogen. In other embodiments, the pathogen or a disease caused by the pathogen is suppressed upon application of the composition compared to a control pathogen lacking the application of the composition. In some embodiments, the method further comprises the step of: introducing at least one additional polynucleotide encoding an inhibitory RNA molecule that are directed to at least one different target sequence in a pathogen. In some embodiments, the suppression lasts at least 1 hour, at least 12 hours, at least 1 day, at least 2 days, at least 3 days, at least 4 days, at least 5 days, at least one week or at least two weeks.

The present disclosure provides a method for preventing, treating or curing a disease or condition in a subject suffering therefrom a pathogen, comprising the steps of: introducing into a minicell at least one polynucleotide encoding an inhibitory RNA molecule that targets a nucleic acid sequence encoding an amino acid sequence at least 90% identical to SEQ ID NO:18, 20, 22 or 24, and applying the minicell to the subject, wherein the inhibitory RNA molecule causes downregulation of said at least one target sequence in the pathogen, and wherein the pathogen or a disease caused by the pathogen is suppressed upon application of the composition compared to a control pathogen lacking the application of the composition.

In addition to the exemplary aspects and embodiments described above, further aspects and embodiments will become apparent by the following descriptions.

### BRIEF DESCRIPTION OF THE FIGURES

FIGS. 1A-1C illustrates characterization and purification of minicells. FIG. 1A illustrates a scanning electron microscopy of a rod-shaped bacterial parental cell and a relatively uniform particle size of minicells (scale bar 1 µm). FIG. 1B illustrates size distribution of the un-purified minicell production batch. Two humps represent different populations composed by smaller anucleated minicells (mean diameter about 0.5 µm or less) and larger replicating parent cells (mean diameter about 1.0 µm). The first peak of the multisizer data represents the minicells and the second peak is for the E. coli parental cells. FIG. 1C illustrates size distribution of the purified minicell production batch. The peak for the parental cells disappeared after the purification step. The size distribution of the purified minicell production shows that only small anucleated minicells are present (purity > 99%).

FIG. 2A illustrates the sequence length of dsRNAs molecules encapsulated into minicells, that is, minicells-encapsulated dsRNAs (ME-dsRNAs) such as ME-CHS3a; ME-CHS3b1; ME-CHS3b2; ME-DCL1; and ME-DCL2. FIG. 2B illustrates treatment of ME-dsRNA and naked-dsRNA, respectively, with the RNase A. The treated and untreated ME-dsRNA were extracted prior to gel electrophoresis.

FIGS. 3A-3F illustrate effects of the minicells-encapsulated dsRNAs (ME-dsRNAs) on mycelial growth and silencing of respective genes in Botrytis cinerea at different time points. FIG. 3A illustrates inhibition of fungal growth in response to single ME-dsRNAs targeting cell wall integrity related-genes chitin synthase 3a (Chs3a), and chitin synthase 3b (Chs3b) and RNAi-machinery related-genes dicer-like protein 1 (DCL1), and dicer-like protein 2 (DCL2) of B. cinerea at different concentrations, respectively. FIG. 3B illustrates inhibition of fungal growth in response to double dsRNAs ((i) CHS3b1 dsRNA and CHS3b2 dsRNA (ME-CHS3b1+2) or (ii) DCL1 dsRNA and DCL2 dsRNA (ME-DCL1+2)) encapsulated by minicells. FIGS. 3C and 3D illustrate mycelial growth inhibition rate (%) in response to ME-dsRNAs, which was quantified from FIGS. 3A and 3B, respectively. FIGS. 3E-3F illustrate relative normalized expressions of the B. cinereal genes in responses to single and double ME-dsRNAs at different time points (24, 48, and 72 hours after ME-dsRNA treatment) FIG. 3E: relative expression of Chs3a and Chs3b;

FIG. 3F: relative expression of DCL1 and DCL2. Data represents the mean ± SEM. Bars labeled with different letters are significantly different at P < 0.05 according to Duncan’s multiple range test. Asterisks indicate significant differences between minicells and ME-dsRNA-treated sample at each time point according to t-test; *P < 0.05, **P < 0.01, ***P < 0.001.

FIG. 4A illustrates the mycelial growth inhibitory activity of the minicells-encapsulated dsRNAs (ME-dsRNAs) targeting various genes of B. cinerea on two other fruit rot causing fungi (Alternaria alternata and Penicillium expansum). Different concentrations of single or double ME-dsRNAs targeting CHS3 a, CHS3b1, CHS3b2, DCL1, and/or DCL2 genes were tested to see effects on mycelial growth of Alternaria alternata (FIGS. 4A and 4C), and Penicillium expansum (FIGS. 4E and 4G) at 72 hours after treatment. The mycelial growth inhibition rate (%) of Alternaria alternata (FIGS. 4B and 4D), and Penicillium expansum (FIGS. 4F and 4H). Data represents the mean ± SEM. Bars labeled with different letters are significantly different at P < 0.05 according to Duncan’s multiple range test.

FIGS. 5A-5C illustrates effectiveness of topically applied minicells-encapsulated dsRNAs (ME-dsRNAs) and naked dsRNAs in inhibiting gray mold diseases under greenhouse conditions. FIG. 5A illustrates schematic representation of applications of ME-dsRNAs and naked-dsRNA on strawberry fruit at 1 hour prior to B. cinerea inoculations. The strawberry fruits were observed from the strawberry plants 5 days after the inoculation for testing disease severity. FIG. 5B illustrates effects of ME-dsRNAs and naked-dsRNA sprayed onto strawberry fruits after 5 days from B. cinerea inoculations, which reduced gray mold disease symptoms and lesion diameter when compared with the application of empty minicells as a control (1st strawberry; left). FIG. 5C illustrates lesion areas affected by B. cinerea, which are quantified from FIG. 5B. ME-dsRNAs and naked-dsRNA sprayed onto fruits at 1 hour prior to B. cinerea inoculations show significantly smaller lesion area 5 days after inoculations when compared with empty minicells (i.e. dsRNAs not encapsulated). Data represent the mean ±SEM. Bars labeled with the different letters are significantly different atP < 0.05 according to Duncan’s multiple range test.

FIGS. 6A-6C illustrates effectiveness of topically applied minicells-encapsulated dsRNAs (ME-dsRNAs) and naked dsRNAs in inhibiting gray mold diseases under greenhouse conditions.

FIG. 6A illustrates schematic representation of applications of ME-dsRNAs and naked-dsRNA on strawberry fruit at 7 days prior to B. cinerea inoculations. The strawberry fruits were observed from the strawberry plants 5 days after the inoculation for testing disease severity. FIG. 6B illustrates effects of ME-dsRNAs and naked-dsRNA sprayed onto strawberry fruits 5 days after B. cinerea inoculations. ME-dsRNAs showed significant reduction of gray mold disease symptoms and lesion diameter when compared with the application of empty minicells and naked-dsRNAs (Naked-CHS3b1+2 and Naked-DCL1+2), respectively. FIG. 6C illustrates lesion areas affected by B. cinerea, which are quantified from FIG. 6B. ME-dsRNAs sprayed onto fruits at 7 days prior to B. cinerea inoculations show significantly smaller lesion area 5 days after inoculations when compared with empty minicells (i.e. dsRNAs not encapsulated). FIGS. 6B and 6C also show ME-dsRNA-sustained protections against gray mold disease when compared to naked-dsRNAs as significantly smaller disease lesion area was observed on B. cinereal inoculated strawberry fruits at day 12 (i.e. 7 days dsRNAs treatment + 5 days post-inoculation). Data represent the mean ±SEM. Bars labeled with the different letters are significantly different atP < 0.05 according to Duncan’s multiple range test.

### DESCRIPTION OF THE INVENTION

Biological pesticides are quickly gaining momentum in the crop protection market, but there are not many, if any, cost-effective solutions on the market that can provide the same protection from fungal pathogens that chemical pesticides do. Botrytis cinerea, for example, causes 10 to 100 billion dollars in damage a year.

A lot of biological fungicides that are commercial or in development have many challenges associated with them. Most biological fungicides are developed as microbial mixtures of various beneficial bacteria, yeast, and fungi. Although they have great lab efficacy, that efficacy is rarely translated into field conditions. The major challenges are standardized production of commercial biocontrol products, developing a product for a multi-host pathogen, and inconsistent field performance due to the harsh effects of environmental pressures.

RNAi is a post-transcription gene regulation mechanism that is present in all known eukaryotes. The cellular RNAi machinery is initiated by dsRNAs that are initially processed into small interfering RNAs (siRNAs) by DCL proteins and eventually leads to the degradation of target mRNAs through the action of the gene silencing complex (RISC). RNAi-based genetic transformation technology has widely been utilized to control several insect pests, and diseases, in what is collectively coined as ‘host-induced gene silencing’ (HIGS) (Fire at al. 1998; Baulcombe et al, 2015). For instance, the expression of dsRNAs targeting dcl½ or target of the rapamycin (TOR) genes of B. cinerea significantly suppressed gray mold disease progression in transgenic Arabidopsis, potato and tomato plants, respectively. However, technical limitations of generating stable transformed plants and the public concern surrounding genetically modified (GM) crops restrict the broad application of HIGS technology to a majority of horticultural crops.

The present disclosure teaches a spray-induced gene silencing (SIGS), which involves the exogenous application of the RNAi as an appealing alternative to HIGS. In some embodiments, SIGS does not incorporate foreign genes in the treated species.

To address the applicability issues associated with sprayable dsRNAs, the present disclosure teaches a dsRNA bioproduction platform that is based on bacterial minicell carrier systems.

The present disclosure relates to systems, compositions and methods for controlling pests or pathogens (e.g. fungal pathogens) by delivering inhibitory RNA molecules to a pathogen or a plant suffering therefrom, or a pathogen-infected plant. The disclosure teaches a minicell platform to deliver one or more inhibitory RNA molecules to a pathogen or a plant suffering therefrom, or a pathogen-infected plant, thereby suppressing expression of target genes associated with pathogen’s growth, lifecycle, metabolism and/or survival. This will lead to improving the sustainability of crop protection from pests and diseases. The disclosure teaches new minicell carriers for biological pesticides (including inhibitory RNA molecules) to ensure a safe, scalable, environment-friendly and cost-effective delivery of biopesticides to plants.

Disclosed herein are compositions and methods for genetic control of plant pathogens and pest infestations. In aspects, one or more genes essential to the lifecycle of a plant pathogen and/or pest are identified as a target gene for RNA interference. In aspects, recombinant vectors encoding inhibitory RNA molecules are designed to suppress, inhibit, repress, control expression of one or more target gene(s) essential for growth, lifecycle, survival, development, and/or reproduction of a plant pathogen and/or pest. In aspects, minicells derived from a parental cells are prepared to efficiently deliver such inhibitory RNA molecules to a subject.

### Definitions

While the following terms are believed to be well understood by one of ordinary skill in the art, the following definitions are set forth to facilitate explanation of the presently disclosed subject matter.

The term “a” or “an” refers to one or more of that entity, i.e. can refer to a plural referents. As such, the terms “a” or “an”, “one or more” and “at least one” are used interchangeably herein. In addition, reference to “an element” by the indefinite article “a” or “an” does not exclude the possibility that more than one of the elements is present, unless the context clearly requires that there is one and only one of the elements.

As used herein, the terms “applying” or “application” of a substance (such as a minicell or minicell-encapsulated dsRNA) taught herein to a subject includes any route of introducing or delivering to a subject a compound, a composition, an agent, a formulation, a platform or a system to perform its intended function. The applying or application can be carried out by any suitable route, including orally, intranasally, parenterally (intravenously, intramuscularly, intraperitoneally, or subcutaneously), rectally, or topically. Applying or application includes self-application, application by another, or application with other ingredients or products.

As used herein, “artificially manipulated” means to move, arrange, operate or control by the hands or by mechanical means or recombinant means, such as by genetic engineering techniques, a host or host cell, so as to produce a host or host cell that has a different biological, biochemical, morphological, or physiological phenotype and/or genotype in comparison to unmanipulated, naturally-occurring counterpart. In some embodiments, genetic engineering methods may rely on the introduction of foreign, not-endogenous nucleic acids, including regulatory elements such as promoters and terminators, and genes that are involved in the expression of a new trait or function as markers for identification and selection of transformants, from viruses, bacteria and plants. In one embodiment, the genetic engineering techniques refers to an “anti-sense” technology encompassing RNA interference, the sequence of native genes is inverted to silence the expression of the gene in a host. In another embodiment, the genetic engineering techniques refers to gene or genome editing techniques such as the ones involving the uses of engineered nuclease to enhance the efficacy and precision of gene editing in combination with oligonucleotides including, but not limited to Zinc Finger Nucleases (ZFN), TAL effector nucleases (TALENs), chemical nucleases, meganucleases, homing nucleases, and clustered regularly interspaced short palindromic repeats (CRISPR)-associated endonuclease Cas9 system (using such as Cas9, Cas12a/Cpf1, Cas13/C2c2, CasX and CasY nucleasesCRISPR-Cas9), which can be used to generate genetic variability and introduce desired traits into a host in a targeted manner. The genetic engineering tools can directly alter, change, edit, or mutate the host’s underlying genetic architecture in a targeted manner, in order to bring about a phenotypic trait of interest.

The term “an biologically active agent,” (synonymous with “an bioactive agent”) indicates that a composition or compound itself has a biological effect, or that it modifies, causes, promotes, enhances, blocks, reduces, limits the production or activity of, or reacts with or binds to an endogenous molecule that has a biological effect. In this disclosure, a biologically active agent includes, but are not limited to, an inhibitory RNA molecule as a biologically pesticidal compound. A “biological effect” may be but is not limited to one that impacts a biological process in/onto a plant; one that impacts a biological process in and/or onto a pest, pathogen or parasite. An active agent may be used in agricultural applications. An active agent acts to cause or stimulate a desired effect upon a plant, an insect, a worm, bacteria, fungi, or virus; one that generates or causes to be generated a detectable signal; and the like. Non-limiting examples of desired effects include, for example, (i) suppressing, inhibiting, limiting, or controlling growth of or killing one or more pests, pathogens or parasites that infect plants and (ii) preventing, treating or curing a disease or condition in a plant suffering from one or more pests, pathogens or parasites. The disclosure teaches that biologically active compositions, complexes or compounds are used in agricultural applications and compositions.

The term “biologically pesticidal compound” or “biological pesticide” or “biopesticide” indicates that the composition, complex or compound has a pesticidal activity that impacts a plant suffering from a disease or disorder in a positive sense and/or impacts a pest, pathogen or parasite in a negative sense. Thus, a biologically pesticidal composition, complex or compound can cause or promote a biological, biochemical, catalytic or metabolic activity to a plant that is detrimental to the growth and/or maintenance of a pest, pathogen or parasite; negatively impact a pest, a pathogen or a parasite that causes a disease or disorder within a host such as a plant.

As used herein the term “biocontrol” or “biological control” refers to control of pests by interference with their ecological status. Successful biological control reduces the population density of the target species. The term “biocontrol” as a biocontrol agent refers to a compound or composition which originates in a biological matter and is effective in treating, preventing, ameliorating, inhibiting, eliminating or delaying the onset of at least one of bacterial, fungal, viral, insect, or any other plant pest infections or infestations and inhibition of spore germination and hyphae growth. It is appreciated that any biocontrol agent is environmentally safe, that it, it is detrimental to the target species, but does not substantially damage other species in a non-specific manner. Furthermore, it is understood that the term “biocontrol agent” or “biocontrol compound” also encompasses the term “biopesticide”, “biological pesticide” or “biologically pesticidal compound”. In this disclosure, biocontrols refer to biologically active compounds such as a nucleic acid encoding an inhibitory RNA biomolecule including, but not limited to, an antisense RNA, a single-stranded RNA (ssRNA), a double-stranded RNA (dsRNA), a hairpin RNA (hpRNA), a short-hairpin RNA (hRNA), a small-interfering RNA (siRNA), a microRNA (miRNA), ribozyme, and aptamer.

As used herein the terms “biopesticide” or “biopesticides” refers to a substance or mixture of substances intended for preventing, destroying or controlling any pest. Specifically, the term relates to substances or mixtures which are effective for treating, preventing, ameliorating, inhibiting, eliminating or delaying the onset of bacterial, fungal, viral, insect- or other pest-related infection or infestation, spore germination and hyphae growth. Also used as substances applied to crops either before or after harvest to protect the commodity from deterioration during storage and transport. As a contraction of ‘biological pesticides’, biopesticides include several types of pest management intervention through predatory, parasitic, or chemical relationships. The term has been associated historically with biological control – and by implication – the manipulation of living organisms. In this disclosure, biopesticides refer to biologically active compounds such as a nucleic acid encoding an inhibitory RNA biomolecule including, but not limited to, an antisense RNA, a single-stranded RNA (ssRNA), a double-stranded RNA (dsRNA), a hairpin RNA (hpRNA), a short-hairpin RNA (shRNA), a small-interfering RNA (siRNA), a microRNA (miRNA), ribozyme, and aptamer.

The term “plant pathogen” or “pathogen” refers to an organism (bacteria, virus, protist, algae or fungi) that infects plants or plant components. Examples include molds, fungi and rot that typically use spores to infect plants or plant components (e.g fruits, vegetables, grains, stems, roots). A “plant pathogen” also includes all genes necessary for the pathogenicity or pathogenic effects in the plant, or that by their suppression or elimination, such effects are reduced or eliminated.

The term “pest” is defined herein as encompassing vectors of plant, humans or livestock disease, unwanted species of bacteria, fungi, viruses, insects, nematodes mites, ticks or any organism causing harm during or otherwise interfering with the production, processing, storage, transport or marketing of food, agricultural commodities, wood and wood products.

The term “subject” can be any singular or plural subject, including, but not limited to plants, crops, vegetables, and herbs. Said subjects can be healthy subjects or any subjects suffering or going to suffer from an disease caused by a pest, pathogen, or parasite. In some embodiments, the subject is a plant. In other embodiments, the subject is a pest, pathogen, or parasite.

The term “plant” or “target plant” includes any plant sustainable to a pathogen. It further includes whole plants, plant organs, progeny of whole plants or plant organs, embryos, somatic embryos, embryo-like structures, protocorms, protocorm-like bodies (PLBs), and suspensions of plant cells. Plant organs comprise, e.g., shoot vegetative organs/structures (e.g., leaves, stems and tubers), roots, flowers and floral organs/structures (e.g., bracts, sepals, petals, stamens, carpels, anthers and ovules), seed (including embryo, endosperm, and seed coat) and fruit (the mature ovary), plant tissue (e.g., vascular tissue, ground tissue, and the like) and cells (e.g., guard cells, egg cells, trichomes and the like). The class of plants that can be used in the disclosure is generally as broad as the class of higher and lower plants amenable to the molecular biology and plant breeding techniques, specifically angiosperms (monocotyledonous (monocots) and dicotyledonous (dicots) plants including eudicots. It includes plants of a variety of ploidy levels, including aneuploid, polyploid, diploid, haploid and hemizygous. The genetically altered plants described herein can be monocot crops, such as, sorghum, maize, wheat, rice, barley, oats, rye, millet, and triticale. The disclosure may also include Cannabaceae and other Cannabis strains, such as C. sativa generally.

Examples of additional plants species of interest include, but are not limited to, corn, wheat, rice, barley, oat, rye, sorghum, millet, sugar cane, strawberry, blueberry, raspberry, blackberry, apple, grape, pear, peach, melon, cucumber, pumpkin, squash, soybean, sugar beet, spinach, swiss chard, potato, eggplant, tomato, sunflower, safflower, gladiolus, cotton, canola, alfalfa, cannabis, Brassica, peanut, tobacco, banana, duckweed, pineapple, date, onion, cashew, pistachio, citrus, rose, almond, coffee, bean, legume, watermelon, squash, cabbage, turnip, mustard, cacti, pecan, flax, sweet potato, coconut, avocado, cantaloupe, vegetables, and herbs.

As used herein the terms “cellular organism” “microorganism” or “microbe” should be taken broadly. These terms are used interchangeably and include, but are not limited to, the two prokaryotic domains, Bacteria and Archaea, as well as certain eukaryotic fungi and protists.

The term “prokaryotes” is art recognized and refers to cells that contain no nucleus or other cell organelles. The prokaryotes are generally classified in one of two domains, the Bacteria and the Archaea. The definitive difference between organisms of the Archaea and Bacteria domains is based on fundamental differences in the nucleotide base sequence in the 16S ribosomal RNA.

The term “Archaea” refers to a categorization of organisms of the division Mendosicutes, typically found in unusual environments and distinguished from the rest of the prokaryotes by several criteria, including the number of ribosomal proteins and the lack of muramic acid in cell walls. On the basis of ssrRNA analysis, the Archaea consist of two phylogenetically-distinct groups: Crenarchaeota and Euryarchaeota. On the basis of their physiology, the Archaea can be organized into three types: methanogens (prokaryotes that produce methane); extreme halophiles (prokaryotes that live at very high concentrations of salt (NaCl); and extreme (hyper) thermophilus (prokaryotes that live at very high temperatures). Besides the unifying archaeal features that distinguish them from Bacteria (i.e., no murein in cell wall, ester-linked membrane lipids, etc.), these prokaryotes exhibit unique structural or biochemical attributes which adapt them to their particular habitats. The Crenarchaeota consists mainly of hyperthermophilic sulfur-dependent prokaryotes and the Euryarchaeota contains the methanogens and extreme halophiles.

“Bacteria” or “eubacteria” refers to a domain of prokaryotic organisms. Bacteria include at least 11 distinct groups as follows: (1) Gram-positive (gram+) bacteria, of which there are two major subdivisions: (1) high G+C group (Actinomycetes, Mycobacteria, Micrococcus, others) (2) low G+C group (Bacillus, Clostridia, Lactobacillus, Staphylococci, Streptococci, Mycoplasmas); (2) Proteobacteria, e.g., Purple photosynthetic+non-photosynthetic Gram-negative bacteria (includes most “common” Gram-negative bacteria); (3) Cyanobacteria, e.g., oxygenic phototrophs; (4) Spirochetes and related species; (5) Planctomyces; (6) Bacteroides, Flavobacteria; (7) Chlamydia; (8) Green sulfur bacteria; (9) Green non-sulfur bacteria (also anaerobic phototrophs); (10) Radioresistant micrococci and relatives; (11) Thermotoga and Thermosipho thermophiles.

A “eukaryote” is any organism whose cells contain a nucleus and other organelles enclosed within membranes. Eukaryotes belong to the taxon Eukarya or Eukaryota. The defining feature that sets eukaryotic cells apart from prokaryotic cells (the aforementioned Bacteria and Archaea) is that they have membrane-bound organelles, especially the nucleus, which contains the genetic material, and is enclosed by the nuclear envelope.

The terms “genetically modified host cell,” “recombinant host cell,” and “recombinant strain” are used interchangeably herein and refer to host cells that have been genetically modified by the cloning and transformation methods of the present disclosure. Thus, the terms include a host cell (e.g., bacteria, yeast cell, fungal cell, CHO, human cell, etc.) that has been genetically altered, modified, or engineered, such that it exhibits an altered, modified, or different genotype and/or phenotype (e.g., when the genetic modification affects coding nucleic acid sequences of the microorganism), as compared to the naturally-occurring organism from which it was derived. It is understood that in some embodiments, the terms refer not only to the particular recombinant host cell in question, but also to the progeny or potential progeny of such a host cell.

The term “wild-type microorganism” or “wild-type host cell” describes a cell that occurs in nature, i.e. a cell that has not been genetically modified. In the disclosure, “wild type strain” or “wild strain” or “wild type cell line” refers to a cell strain/line that can produce minicells. In some embodiments, wild type bacterial strains and/or cell lines such as E. coli strain p678-54 and B. subtilis strain CU403 can make miniature cells deficient in chromosomal DNA. Methods for producing such minicells are known in the art. See, for example, Adler et al., 1967, Proc. Natl. Acad. Sci. USA 57:321-326; Inselburg J, 1970 J. Bacteriol. 102(3):642-647; Frazer 1975, Curr. Topics Microbiol. Immunol. 69:1-84, Reeve et al 1973, J. Bacteriol. 114(2):860-873; and Mendelson et al 1974 J. Bacteriol. 117(3):1312-1319.

The term “genetically engineered” may refer to any manipulation of a host cell’s genome (e.g. by insertion, deletion, mutation, or replacement of nucleic acids).

The term “control” or “control host cell” refers to an appropriate comparator host cell for determining the effect of a genetic modification or experimental treatment. In some embodiments, the control host cell is a wild type cell. A control host cell is genetically identical to the genetically modified host cell, save for the genetic modification(s) differentiating the treatment host cell. In other embodiments, the control minicell is an empty minicell without any biologically active agent present.

As used herein, the term “genetically linked” refers to two or more traits that are co-inherited at a high rate during breeding such that they are difficult to separate through crossing.

A “recombination” or “recombination event” as used herein refers to a chromosomal crossing over or independent assortment.

As used herein, the term “phenotype” refers to the observable characteristics of an individual cell, cell culture, organism, or group of organisms which results from the interaction between that individual’s genetic makeup (i.e., genotype) and the environment.

As used herein, the term “chimeric” or “recombinant” when describing a nucleic acid sequence or a protein sequence refers to a nucleic acid, or a protein sequence, that links at least two heterologous polynucleotides, or two heterologous polypeptides, into a single macromolecule, or that rearranges one or more elements of at least one natural nucleic acid or protein sequence. For example, the term “recombinant” can refer to an artificial combination of two otherwise separated segments of sequence, e.g., by chemical synthesis or by the manipulation of isolated segments of nucleic acids by genetic engineering techniques known in the art.

As used herein, a “synthetic nucleotide sequence” or “synthetic polynucleotide sequence” is a nucleotide sequence that is not known to occur in nature or that is not naturally occurring. Generally, such a synthetic nucleotide sequence will comprise at least one nucleotide difference when compared to any other naturally occurring nucleotide sequence.

As used herein, a “synthetic amino acid sequence” or “synthetic peptide” or “synthetic protein” is an amino acid sequence that is not known to occur in nature or that is not naturally occurring. Generally, such a synthetic protein sequence will comprise at least one amino acid difference when compared to any other naturally occurring protein sequence.

As used herein, the term “nucleic acid” refers to a polymeric form of nucleotides of any length, either ribonucleotides or deoxyribonucleotides, or analogs thereof. This term refers to the primary structure of the molecule, and thus includes double- and single-stranded DNA, as well as double- and single-stranded RNA. It also includes modified nucleic acids such as methylated and/or capped nucleic acids, nucleic acids containing modified bases, backbone modifications, and the like. The terms “nucleic acid” and “nucleotide sequence” are used interchangeably.

As used herein, the term “gene” refers to any segment of DNA associated with a biological function. Thus, genes include, but are not limited to, coding sequences and/or the regulatory sequences required for their expression. Genes can also include non-expressed DNA segments that, for example, form recognition sequences for other proteins. Genes can be obtained from a variety of sources, including cloning from a source of interest or synthesizing from known or predicted sequence information, and may include sequences designed to have desired parameters.

As used herein, the term “homologous” or “homologue” or “ortholog” is known in the art and refers to related sequences that share a common ancestor or family member and are determined based on the degree of sequence identity. For example, homologous sequences may have from about 70%-100, or more generally 80% to 100% sequence identity, such as about 81%; about 82%; about 83%; about 84%; about 85%; about 86%; about 87%; about 88%; about 89%; about 90%; about 91%; about 92%; about 93%; about 94% about 95%; about 96%; about 97%; about 98%; about 98.5%; about 99%; about 99.5%; and about 100%. The property of substantial homology is closely related to specific hybridization. For example, a nucleic acid molecule is specifically hybridizable when there is a sufficient degree of complementarity to avoid non-specific binding of the nucleic acid to non-target sequences under conditions where specific binding is desired, for example, under stringent hybridization conditions. The terms “homology,” “homologous,” “substantially similar” and “corresponding substantially” are used interchangeably herein. They refer to nucleic acid fragments wherein changes in one or more nucleotide bases do not affect the ability of the nucleic acid fragment to mediate gene expression or produce a certain phenotype. These terms also refer to modifications of the nucleic acid fragments of the instant disclosure such as deletion or insertion of one or more nucleotides that do not substantially alter the functional properties of the resulting nucleic acid fragment relative to the initial, unmodified fragment. It is therefore understood, as those skilled in the art will appreciate, that the disclosure encompasses more than the specific exemplary sequences. These terms describe the relationship between a gene found in one species, subspecies, variety, cultivar or strain and the corresponding or equivalent gene in another species, subspecies, variety, cultivar or strain. For purposes of this disclosure homologous sequences are compared. “Homologous sequences” or “homologues” or “orthologs” are thought, believed, or known to be functionally related. A functional relationship may be indicated in any one of a number of ways, including, but not limited to: (a) degree of sequence identity and/or (b) the same or similar biological function. Also, both (a) and (b) are indicated. Homology can be determined using software programs readily available in the art, such as those discussed in Current Protocols in Molecular Biology (F.M. Ausubel et al., eds., 1987) Supplement 30, section 7.718, Table 7.71. An example of a local alignment algorithm utilized for the comparison of sequences is the NCBI Basic Local Alignment Search Tool (BLAST®) (Altschul et al. 1990 J. Mol. Biol. 215: 403-10), which is available from several sources, including the National Center for Biotechnology Information (NCBI, Bethesda, Md.) and on the Internet, for use in connection with the sequence analysis programs blastp, blastn, blastx, tblastn and tblastx. It can be accessed on the internet via the National Library of Medicine (NLM)′s world-wide-web URL. A description of how to determine sequence identity using this program is available at the NLM’s website on BLAST tutorial. Another example of a mathematical algorithm utilized for the global comparison of sequences is the Clustal W and Clustal X (Larkin et al. 2007 Bioinformatics, 23, 2947-294, Clustal W and Clustal X version 2.0) as well as Clustal omega. Unless otherwise stated, references to sequence identity used herein refer to the NCBI Basic Local Alignment Search Tool (BLAST®).

As used herein, the term “endogenous” or “endogenous gene,” refers to the naturally occurring gene, in the location in which it is naturally found within the host cell genome. In the context of the present disclosure, operably linking a heterologous promoter to an endogenous gene means genetically inserting a heterologous promoter sequence in front of an existing gene, in the location where that gene is naturally present. An endogenous gene as described herein can include alleles of naturally occurring genes that have been mutated.

As used herein, the term “exogenous” is used interchangeably with the term “heterologous,” and refers to a substance coming from some source other than its native source. For example, the terms “exogenous protein,” or “exogenous gene” refer to a protein or gene from a non-native source or location, and that have been artificially supplied to a biological system.

As used herein, “promoter” refers to a DNA sequence capable of controlling the expression of a coding sequence or functional RNA. A promoter may be operably linked to a coding sequence for expression in a cell, or a promoter may be operably linked to a nucleotide sequence encoding a signal sequence which may be operably linked to a coding sequence for expression in a cell. A “plant promoter” may be a promoter capable of initiating transcription in plant cells. Examples of promoters under developmental control include promoters that preferentially initiate transcription in certain tissues, such as leaves, roots, seeds, fibers, xylem vessels, tracheids, or sclerenchyma. Such promoters are referred to as “tissue-preferred.” Promoters which initiate transcription only in certain tissues are referred to as “tissue-specific.” A “cell type-specific” promoter primarily drives expression in certain cell types in one or more organs, for example, vascular cells in roots or leaves. An “inducible” promoter may be a promoter which may be under environmental control. Examples of environmental conditions that may initiate transcription by inducible promoters include anaerobic conditions and the presence of light. Tissue-specific, tissue-preferred, cell type specific, and inducible promoters constitute the class of “non-constitutive” promoters. A “constitutive” promoter is a promoter which may be active under most environmental conditions or in most cell or tissue types. In some embodiments, the promoter sequence consists of proximal and more distal upstream elements, the latter elements often referred to as enhancers. Accordingly, an “enhancer” is a DNA sequence that can stimulate promoter activity, and may be an innate element of the promoter or a heterologous element inserted to enhance the level or tissue specificity of a promoter. Promoters may be derived in their entirety from a native gene, or be composed of different elements derived from different promoters found in nature, or even comprise synthetic DNA segments. It is understood by those skilled in the art that different promoters may direct the expression of a gene in different tissues or cell types, or at different stages of development, or in response to different environmental conditions. It is further recognized that since in most cases the exact boundaries of regulatory sequences have not been completely defined, DNA fragments of some variation may have identical promoter activity.

As used herein, the phrases “recombinant construct”, “expression construct”, “chimeric construct”, “construct”, and “recombinant DNA construct” are used interchangeably herein. Also, “construct”, “vector”, and “plasmid” are used interchangeably herein. A recombinant construct comprises an exogenous, heterologous expression cassette such an artificial combination of nucleic acid fragments, e.g., regulatory and coding sequences that are not found together in nature. In some embodiments, the heterologous expression cassette contains a promoter operably linked to at least one polynucleotide of interest (such as a dsRNA target sequence in the present disclosure). For example, a chimeric construct may comprise regulatory sequences and coding sequences that are derived from different sources, or regulatory sequences and coding sequences derived from the same source, but arranged in a manner different than that found in nature. Such construct may be used by itself or may be used in conjunction with a vector. If a vector is used then the choice of vector is dependent upon the method that will be used to transform host cells. For example, a plasmid vector can be used. The skilled artisan is well aware of the genetic elements that must be present on the vector in order to successfully transform, select and propagate host cells comprising any of the isolated nucleic acid fragments of the disclosure. The skilled artisan will also recognize that different independent transformation events will result in different levels and patterns of expression (Jones et al., (1985) EMBO J. 4:2411-2418; De Almeida et al.,

Mol. Gen. Genetics 218:78-86), and thus that multiple events must be screened in order to obtain lines displaying the desired expression level and pattern. Such screening may be accomplished by Southern analysis of DNA, Northern analysis of mRNA expression, immunoblotting analysis of protein expression, or phenotypic analysis, among others. Vectors can be plasmids, viruses, bacteriophages, pro-viruses, phagemids, transposons, artificial chromosomes, and the like, that replicate autonomously or can integrate into a chromosome of a host cell. A vector can also be a naked RNA polynucleotide, a naked DNA polynucleotide, a polynucleotide composed of both DNA and RNA within the same strand, a poly-lysine-conjugated DNA or RNA, a peptide-conjugated DNA or RNA, a liposome-conjugated DNA, or the like, that is not autonomously replicating. As used herein, the term “expression” refers to the production of a functional end-product e.g., an mRNA or a protein (precursor or mature).

“Operably linked” means in this context the sequential arrangement of the promoter polynucleotide according to the disclosure with a further oligo- or polynucleotide, resulting in transcription of said further polynucleotide.

As used herein, the term “transformation” or “transform” refers to the transfer of one or more nucleic acid molecule(s) into a cell. In some embodiments, the term “transformation” or “transform” encompasses all techniques by which a nucleic acid molecule can be introduced into a minicell.

The term “suppression”, “repression”, “silencing” or “inhibition” is used interchangeably to denote the down-regulation of the expression of the product of a target sequence relative to its normal expression level in a wild type organism. Suppression includes expression that is decreased by about 5%, 10%, 15%, 20%, 25%, 30%, 35%, 40%, 45%, 50%, 55%, 60%, 65%, 70%, 75%, 80%, 85%, 90%, 95%, or 100% relative to the wild type expression level. An “effective amount” is an amount of inhibitory RNA sufficient to result in suppression or inhibition of a plant pathogen.

As used herein, the term “protease-deficient strain” refers to a strain that is deficient in one or more endogenous proteases. For example, protease deficiency can be created by deleting, removing, knock-out, silencing, suppressing, or otherwise downregulating at lease on endogenous protease. Said proteases can include catastrophic proteases. For example, BL21 (DE3) E. coli strain is deficient in proteases Lon and OmpT. E. coli strain has cytoplasmic proteases and membrane proteases that can significantly decrease protein production and localization to the membrane. In some embodiments, a protease-deficient strain can maximize production and localization of a protein of interest to the membrane of the cell. “Protease-deficient” can be interchangeably used as “protease-free” in the present disclosure.

As used herein, the term “ribonuclease-deficient strain” refers to a strain that is deficient in one or more endogenous ribonuclease. For example, ribonuclease deficiency can be created by deleting, removing, knock-out, silencing, suppressing, or otherwise downregulating at lease on endogenous ribonuclease. Said ribonuclease can include ribonuclease III. For example, HT115 E. coli strain is deficient in RNase III. In some embodiments, a ribonuclease-deficient strain is unable to and/or has a reduced capability of recognizing dsRNA and cleaving it at specific targeted locations. “Ribonuclease-deficient” can be interchangeably used as “ribonuclease-free” in the present disclosure.

As used herein, the term “anucleate cell” refers to a cell that lacks a nucleus and also lacks chromosomal DNA and which can also be termed as an “anucleate cell”. Because eubacterial and archaebacterial cells, unlike eukaryotic cells, naturally do not have a nucleus (a distinct organelle that contains chromosomes), these non-eukaryotic cells are of course more accurately described as being “without chromosomes” or “achromosomal.” Nonetheless, those skilled in the art often use the term “anucleate” when referring to bacterial minicells in addition to other eukaryotic minicells. Accordingly, in the present disclosure, the term “minicells” encompasses derivatives of eubacterial cells that lack a chromosome; derivatives of archaebacterial cells that lack their chromosome(s), and anucleate derivatives of eukaryotic cells that lack a nucleus and consequently a chromosome. Thus, in the present disclosure, “anucleated cell” or “anucleate cell” can be interchangeably used with the term “achromosomal cell.”

As used herein, “carrier,” “suitable carrier,” or “agriculturally suitable carrier” refers to a diluent, adjuvant, excipient, or vehicle with which a composition can be applied to its target or subject, which does not detrimentally effect the composition.

As used herein, “plants” and “plant derivatives” can refer to any portion of a growing plant, including the roots, stems, stalks, leaves, branches, seeds, flowers, fruits, and the like.

### Overview

The preset disclosure provides a minicell platform, which is a highly modular and tunable biological microcapsule that can encapsulate, stabilize, and effectively deliver a bioactive agents to a subject for controlling a pest or a pathogen. The key to the minicell technology is that it harnesses the capabilities of synthetic biology to produce a bioencapsulation technology that is environmentally compatible, modular in its functionality, and scalable for agricultural applications. Active, non-pathogenic microbial cells are engineered to produce a bioparticle through asymmetric cell division. These bioparticles are small (less than 1 µm in diameter; about 0.5 µm in average), spherical versions of their parent microbial cells and they maintain the properties of the parent cell with a major difference: they lack chromosomal DNA. In this disclosure, the minicell also loses RNaseIII activity or have a suppressed RNaseIII activity for delivering RNA molecules to a subject. Therefore, the biological particles retain the benefits of the parent microbe except for small in size and/or lack of RNaseIII activity, but do not risk contaminating the environment with modified DNA or outcompeting native species since they do not propagate.

In some embodiments, the minicells taught herein are naturally occurring anucleate cells.

In other embodiments, the present disclose teaches novel minicells that are derived from a parental cell that is genetically engineered to form a minicell with RNaseIII activity deficient or reduced. In further embodiments, the present disclose teaches novel minicells that are derived from a parental cell that is genetically engineered to form a minicell with protease activity deficient or reduced. In some embodiments, the present disclose teaches novel minicells that are derived from a parental cell that is genetically engineered to form a minicell with both RNaseIII and protease activity deficient or reduced.

In some embodiments, the minicell can exogenously encapsulate high-payload capacities of biocontrols (e.g. inhibitory RNA molecules). In other embodiments, In some embodiments, the minicell can have the inhibitory RNA molecules expressed from a recombinant vector, and the minicell deliver the encapsulated inhibitory RNA molecules to a subject.

The present disclosure further provides that the minicells taught herein serves as a carrier that protects biocontrol agents/ingredients from environmental stresses until it delivers its high-payload capacity to a subject through the natural breakdown of its biodegradable membrane. This bioencapsulation technology overcomes many of the problems of bioactive agent delivery (including single-stranded RNA or double-stranded RNA) and can serve as the much-needed replacement to traditional techniques using plastic microcapsules.

In some embodiments, the minicell technology can be engineered in various ways to improve stability of biocontrol agents (such as inhibitory RNA molecules) encapsulated into the minicell and provide extended release profiles of the biocontrol agents.

### Minicells

Minicells are bacterially-derived achromosomal microparticles, generally produced as a result of aberrant cell divisions. Similar to the parental cells, minicells contain membranes, ribosomes, RNA and proteins; but unlike normal cells, they cannot divide or grow (Farley et al 2016). That is, minicells are the result of aberrant, asymmetric cell division, and contain membranes, peptidoglycan, ribosomes, RNA, protein, and often plasmids but no chromosome. Because minicells lack chromosomal DNA, minicells cannot divide or grow, but they can continue other cellular processes, such as ATP synthesis, replication and transcription of plasmid DNA, and translation of mRNA. Although chromosomes do not segregate into minicells, extrachromosomal and/or episomal genetic expression elements may segregate, or may be introduced into minicells after segregation from parent cells.

The disclosure teaches that minicells are derived from bacterial parental cells whose cell division is impaired by naturally-occurring spontaneous mutation(s) or artificially manipulated mutations by genetic engineering techniques known in the art.

The disclosure teaches that minicells are derived from bacterial parental cells whose RNaseIII activity is deficient by naturally-occurring spontaneous mutation(s) or artificially manipulated mutations by genetic engineering techniques known in the art.

The disclosure teaches that minicells are derived from bacterial parental cells whose protease activity is deficient by naturally-occurring spontaneous mutation(s) or artificially manipulated mutations by genetic engineering techniques known in the art.

The disclosure teaches that minicells are derived from bacterial parental cells whose cell division is impaired and RNaseIII activity is deficient by naturally-occurring spontaneous mutation(s) or artificially manipulated mutations by genetic engineering techniques known in the art.

The disclosure teaches that minicells are derived from bacterial parental cells whose cell division is impaired and protease activity is deficient by naturally-occurring spontaneous mutation(s) or artificially manipulated mutations by genetic engineering techniques known in the art.

The disclosure teaches that minicells are derived from bacterial parental cells whose cell division is impaired and both RNaseIII and protease activities are deficient by naturally-occurring spontaneous mutation(s) or artificially manipulated mutations by genetic engineering techniques known in the art.

The disclosure teaches that minicells are derived from a E. coli parental cell that is genetically engineered to have any one of minCDE genes mutated and/or rnc gene mutated.

The disclosure teaches that minicells are derived from a E. coli parental cell that is genetically engineered to have any one of minCDE genes mutated and/or gene(s) encoding protease(s) mutated.

The disclosure teaches that minicells are derived from a E. coli parental cell that is genetically engineered to have any one of minCDE genes mutated, rnc gene mutated and/or gene(s) encoding protease(s) mutated.

### Eubacterial Minicells

One type of minicell is a eubacterial minicell. When DNA replication and/or chromosomal partitioning is altered, membrane-bounded vesicles “pinch off” from parent cells before transfer of chromosomal DNA is completed. As a result of this type of dysfunctional division, minicells are produced which contain an intact outer membrane, inner membrane, cell wall, and all of the cytoplasm components but do not contain chromosomal DNA.

In some embodiments, the bacterially-derived minicells are produced from a strain, including, but are not limited to a strain of Escherichia coli, Bacillus spp., Salmonella spp., Listeria spp., Mycobacterium spp., Shigella spp., or Yersinia spp. In some embodiments, the bacterially-derived minicells are produced from a strain that naturally produces minicells. Such natural minicell producing strains produce minicells, for example, at a 2:1 ratio (2 bacterial cells for every one minicell). In certain embodiments, exemplary bacterial strains that naturally produce minicells include, but are not limited to E. coli strain number P678-54, Coli Genetic Stock Center (CGSC) number: 4928 and B. subtilis strain CU403.

As one example, mutations in B. subtilis smc genes result in the production of minicells (Britton et al., 1998, Genes and Dev. 12:1254-1259; Moriya et al., 1998, Mol Microbiol 29:179-87). Disruption of smc genes in various cells is predicted to result in minicell production therefrom.

As another example, mutations in the divIVA gene of Bacillus subtilis results in minicell production. When expressed in E. coli, B. subtilis or yeast Schizosaccharomyces pombe, a DivIVA-GFP protein is targeted to cell division sites therein, even though clear homologs of DivIVA do not seem to exist in E. coli, B. subtilis or S. pombe (David et al., 2000, EMBO J. 19:2719-2727. Over- or under-expression of B. subtilis DivIVA or a homolog thereof may be used to reduce minicell production in a variety of cells.

In some embodiments, the minicell-producing bacteria is a Gram-negative bacteria. The Gram-negative bacteria includes, but is not limited to, Escherichia coli, Salmonella spp. including Salmonella typhimurium, Shigella spp. including Shigella flexneri, Pseudomonas aeruginosa, Agrobacterium, Campylobacter jejuni, Lactobacillus spp., Neisseria gonorrhoeae, and Legionella pneumophila,. In some embodiments, the minicell-producing gram-negative bacteria can produce minicells naturally caused by endogenous or exogenous mutation(s) associated with cell division and/or chromosomal partitioning. In some embodiments, the minicell-producing bacteria comprises endogenous or exogenous gene(s) that is involved in cell division and/or chromosomal partitioning, where the gene is genetically modified such as by homologous recombination, compared to a corresponding wild-type gene. In some embodiments, the minicell-producing gram-negative bacteria is deficient in protease and/or its activity naturally and/or by genetic engineering techniques disclosed herein. In some embodiments, the protease-deficient minicell-producing gram-negative bacteria comprises a recombinant expression vector comprising a gene or genes that is involved in a protein of interest disclosed in the present disclosure.

In some embodiments, the minicell-producing bacteria can be a Gram-positive bacteria. The Gram-positive bacteria includes, but is not limited to, Bacillus subtilis, Bacillus cereus, Corynebacterium Glutamicum, Lactobacillus acidophilus, Staphylococcus spp., or Streptococcus spp. In some embodiments, the minicell-producing gram-positive bacteria can produce minicells naturally caused by endogenous or exogenous mutation(s) associated with cell division and/or chromosomal partitioning. In some embodiments, the minicell-producing gram-positive bacteria comprises endogenous or exogenous gene(s) that is involved in cell division and/or chromosomal partitioning, where the gene is genetically modified such as by homologous recombination, compared to a corresponding wild-type gene. In some embodiments, the minicell-producing gram-positive bacteria is deficient in protease and/or its activity naturally and/or by genetic engineering techniques disclosed herein. In some embodiments, the protease-deficient minicell-producing gram-positive bacteria comprises a recombinant expression vector comprising a gene or genes that is involved in a protein of interest disclosed in the present disclosure.

The minicell-producing bacteria can be a Extremophilic bacteria. The Extremophilic bacteria includes, but is not limited to, Thermophiles including Thermus aquaticus, Psychrophiles, Piezophiles, Halophilic bacteria, Acidophile, Alkaliphile, Anaerobe, Lithoautotroph, Oligotroph, Metallotolerant, Oligotroph, Xerophil or Polyextremophile. In some embodiments, the minicell-producing Extremophilic bacteria can produce minicells naturally caused by endogenous or exogenous mutation(s) associated with cell division and/or chromosomal partitioning. In some embodiments, the minicell-producing Extremophilic bacteria comprises endogenous or exogenous gene(s) that is involved in cell division and/or chromosomal partitioning, where the gene is genetically modified such as by homologous recombination, compared to a corresponding wild-type gene. In some embodiments, the minicell-producing Extremophilic bacteria is deficient in protease and/or its activity naturally and/or by genetic engineering techniques disclosed herein. In some embodiments, the protease-deficient minicell-producing Extremophilic bacteria comprises a recombinant expression vector comprising a gene or genes that is involved in a protein of interest disclosed in the present disclosure.

### Eukaryotic Minicells

Achromosomal eukaryotic minicells (i.e., anucleate cells) are within the scope of the disclosure. Yeast cells are used to generate fungal minicells. See, e.g., Lee et al., Ibd1p, a possible spindle pole body associated protein, regulates nuclear division and bud separation in Saccharomyces cerevisiae, Biochim Biophys Acta 3:239-253, 1999; Kopecka et al., A method of isolating anucleate yeast protoplasts unable to synthesize the glucan fibrillar component of the wall J Gen Microbiol 81:111-120, 1974; and Yoo et al., Fission yeast Hrp1, a chromodomain ATPase, is required for proper chromosome segregation and its overexpression interferes with chromatin condensation, Nucl Acids Res 28:2004-2011, 2000. Cell division in yeast is reviewed by Gould and Simanis, The control of septum formation in fission yeast, Genes & Dev 11:2939-51, 1997).

In some embodiments, the eukaryotic minicells can be produced from yeast cells, such as Saccharomyces cerevisiae, Pichia pastoris and/or Schizosaccharomyces pombe.

As one example, mutations in the yeast genes encoding TRF topoisomerases result in the production of minicells, and a human homolog of yeast TRF genes has been stated to exist (Castano et al., 1996, Nucleic Acids Res 24:2404-10). Mutations in a yeast chromodomain ATPase, Hrp1, result in abnormal chromosomal segregation; (Yoo et al., 2000 Nuc. Acids Res. 28:2004-2011). Disruption of TRF and/or Hrp1 function is predicted to cause minicell production in various cells. Genes involved in septum formation in fission yeast (see, e.g., Gould et al., 1997 Genes and Dev. 11:2939-2951) can be used in like fashion.

Platelets are a non-limiting example of eukaryotic minicells. Platelets are anucleate cells with little or no capacity for de novo protein synthesis. The tight regulation of protein synthesis in platelets (Smith et al., 1999, Vasc Med 4:165-72) may allow for the over-production of exogenous proteins and, at the same time, under-production of endogenous proteins. Thrombin-activated expression elements such as those that are associated with Bcl-3 (Weyrich et al., Signal-dependent translation of a regulatory protein, Bcl-3, in activated human platelets, Cel Biology 95:5556-5561, 1998) may be used to modulate the expresion of exogneous genes in platelets.

As another non-limiting example, eukaryotic minicells are generated from tumor cell lines (Gyongyossy-Issa and Khachatourians, Tumour minicells: single, large vesicles released from cultured mastocytoma cells (1985) Tissue Cell 17:801-809; Melton, Cell fusion-induced mouse neuroblastomas HPRT revertants with variant enzyme and elevated HPRT protein levels (1981) Somatic Cell Genet. 7: 331-344).

Yeast cells are used to generate fungal minicells. See, e.g., Lee et al., Ibd1p, a possible spindle pole body associated protein, regulates nuclear division and bud separation in Saccharomyces cerevisiae, Biochim Biophys Acta 3:239-253, 1999; Kopecka et al., A method of isolating anucleate yeast protoplasts unable to synthesize the glucan fibrillar component of the wall J Gen Microbiol 81:111-120, 1974; and Yoo et al., Fission yeast Hrp1, a chromodomain ATPase, is required for proper chromosome segregation and its overexpression interferes with chromatin condensation, Nucl Acids Res 28:2004-2011, 2000. Cell division in yeast is reviewed by Gould and Simanis, The control of septum formation in fission yeast, Genes & Dev 11:2939-51, 1997). In some embodiments, the present disclosure teaches production of yeast minicells.

### Archaebacterial Minicells

The term “archaebacterium” is defined as is used in the art and includes extreme thermophiles and other Archaea (Woese, C.R., L. Magrum. G. Fox. 1978. Archaebacteria. Journal of Molecular Evolution. 11:245-252). Three types of Archaebacteria are halophiles, thermophiles and methanogens. By physiological definition, the Archaea (informally, archaes) are single-cell extreme thermophiles (including thermoacidophiles), sulfate reducers, methanogens, and extreme halophiles. The thermophilic members of the Archaea include the most thermophilic organisms cultivated in the laboratory. The aerobic thermophiles are also acidophilic; they oxidize sulfur in their environment to sulfuric acid. The extreme halophiles are aerobic or microaerophilic and include the most salt tolerant organisms known. The sulfate-reducing Archaea reduce sulfate to sulfide in extreme environment. Methanogens are strict anaerobes, yet they gave rise to at least two separate aerobic groups: the halophiles and a thermoacidophilic lineage. Non-limiting examples of halophiles include Halobacterium cutirubrum and Halogerax mediterranei. Non-limiting examples of methanogens include Methanococcus voltae; Methanococcus vanniela; Methanobacterium thermoautotrophicum; Methanococcus voltae; Methanothermus fervidus; and Methanosarcina barkeri. Non-limiting examples of thermophiles include Azotobacter vinelandii; Thermoplasma acidophilum; Pyrococcus horikoshii; Pyrococcus furiosus; and Crenarchaeota (extremely thermophilic archaebacteria) species such as Sulfolobus solfataricus and Sulfolobus acidocaldarius.

Archaebacterial minicells are within the scope of the disclosure. Archaebacteria have homologs of eubacterial minicell genes and proteins, such as the MinD polypeptide from Pyrococcus furiosus (Hayashi et al., EMBO J. 20:1819-28, 2001). It is thus possible to create Archaebacterial minicells by methods such as, by way of non-limiting example, overexpressing the product of a min gene isolated from a prokaryote or an archaebacterium; or by disrupting expression of a min gene in an archaebacterium of interest by, e.g., the introduction of mutations thereof or antisense molecules thereto. See, e.g., Laurence et al., Genetics 152:1315-1323, 1999.

By physiological definition, the Archaea (informally, archaes) are single-cell extreme thermophiles (including thermoacidophiles), sulfate reducers, methanogens, and extreme halophiles. The thermophilic members of the Archaea include the most thermophilic organisms cultivated in the laboratory. The aerobic thermophiles are also acidophilic; they oxidize sulfur in their environment to sulfuric acid. The extreme halophiles are aerobic or microaerophilic and include the most salt tolerant organisms known. The sulfate-reducing Archaea reduce sulfate to sulfide in extreme environment. Methanogens are strict anaerobes, yet they gave rise to at least two separate aerobic groups: the halophiles and a thermoacidophilic lineage. In some embodiments, the present disclosure teaches production of archaeal minicells.

### Bacterial Minicell Production

Minicells are produced by parent cells having a mutation in, and/or overexpressing, or under expressing a gene involved in cell division and/or chromosomal partitioning, or from parent cells that have been exposed to certain conditions, that result in aberrant fission of bacterial cells and/or partitioning in abnormal chromosomal segregation during cellular fission (division). The term “parent cells” or “parental cells” refers to the cells from which minicells are produced. Minicells, most of which lack chromosomal DNA (Mulder et al., Mol Gen Genet, 221: 87-93, 1990), are generally, but need not be, smaller than their parent cells.

Minicells are achromosomal, membrane-encapsulated biological nanoparticles that are formed by bacteria following a disruption in the normal division apparatus of bacterial cells. In essence, minicells are small, metabolically active replicas of normal bacterial cells with the exception that they contain no chromosomal DNA and as such, are non-dividing and non-viable. Although minicells do not contain chromosomal DNA, the ability of plasmids, RNA, native and/or recombinantly expressed proteins, and other metabolites have all been shown to segregate into minicells. Some methods of construction of minicell-producing bacterial strains are discussed in detail in U.S. Pat. Application Ser. No. 10/154,951(U.S. Publication No. US/2003/0194798 A1), which is hereby incorporated by reference in its entirety.

Disruptions in the coordination between chromosome replication and cell division lead to minicell formation from the polar region of most rod-shaped prokaryotes. Disruption of the coordination between chromosome replication and cell division can be facilitated through the overexpression of some of the genes involved in septum formation and binary fission. Alternatively, minicells can be produced in strains that harbor mutations in genes that modulate septum formation and binary fission. Impaired chromosome segregation mechanisms can also lead to minicell formation as has been shown in many different prokaryotes.

A description of methods of making, producing, and purifying bacterial minicells can be found, for example, in International Patent application No. WO2018/201160, WO2018/201161, and WO2019/060903, which are incorporated herein by reference.

Also, a description of strains for producing minicells an be found, for example, in International Patent application No. WO2018/201160, WO2018/201161, and WO2019/060903, which are incorporated herein by reference.

In some embodiments, the present disclosure teaches a composition comprising: a minicell and an active agent. In some embodiments, the minicell is derived from a bacterial cell. In some embodiments, the minicell is less than or equal to 1 µm in diameter. The minicell is about 10 nm – about 1000 nm in size, about 20 nm - about 900 nm in size, about 30 nm – about 800 nm in size, about 400 nm – about 700 nm in size, about 50 nm – about 600 nm in size, about 60 nm – about 500 nm in size, about 70 nm – about 550 nm in size, about 80 nm – about 500 nm in size, about 90 nm – about 450 nm in size, about 100 nm – about 400 nm in size, and about 100 nm – about 300 nm in size. In other embodiments, the minicell is about 500 nm in size or less.

In some embodiments, minicell production can be achieved by the overexpression or mutation of genes involved in the segregation of nascent chromosomes into daughter cells. For example, mutations in the parC or mukB loci of E. coli have been demonstrated to produce minicells. The overexpression or mutation of a cell division gene capable of driving minicell production in one family member, can be used to produce minicells in another. For example, it has been shown that the overexpression E. coli ftsZ gene in other Enterobacteriacea family members such as Salmonella spp. and Shigella spp as well as other class members such as Pseudomonas spp. will result in similar levels of minicell production.

In some embodiments, minicells can be produced in E. coli by the overproduction of the protein FtsZ which is an essential component of the Min division system by which E. coli operates. Overproduction of this protein in E. coli results in the inability for this ring to be spatially restricted to the midsection of the cell, thus resulting in production of minicells upon cell division. Because the overproduction of FtsZ can create minicells, it can be overexpressed using a plasmid based system.

The same can be demonstrated in the mutation-based minicell producing bacterial strains. For example, deletion of the Min locus in any of bacterial strains results in minicell production. Cell division genes in which mutation can lead to minicell formation include but are not limited to the min genes (such as minC, minD, and minE).

In some embodiments, E. coli rely on the min system in order to ensure proper replication of parent cells into daughter cells. This min system (known as the minB operon) consists of 3 parts, minD, minC, and minE. These genes work together in order to control the placement of the Z-ring which is comprised of polymerized FtsZ protein. MinC consists of two distinct domains, both of which interact directly with the FtsZ protein in order to inhibit polymerization (Z-ring formation). MinD is a protein that is associated with the membrane that forms at one of the cell’s poles and polymerizes toward the cell’s mid-point. It binds MinC which is distributed throughout the cytoplasm. MinE is a protein that binds to MinD as well and releases MinC. It polymerizes into a ring like shape and oscillates from pole to pole in the cell.

In some embodiments, this system can be manipulated in order to shift the Z-ring to a polar end of the cell which excludes the nucleoid DNA upon completion of replication. The Z-ring can be shifted by not allowing the cell to sequester MinC to the polar ends of the cell. In the absence of MinC or MinD, or overexpression of MinE, E. coli cells will form achromosomal and/or anucleate cells. The FtsZ and the Min systems for causing asymmetrical cell division are exemplified by Piet et al, 1990, Proc. Natl. Acad. Sci. USA 87:1129-1133 and Xuan-Chuan et al, 2000, J. Bacteriol. 182(21):6203-62138, each of which is incorporated herein by reference.

Genes can be introduced in a site directed fashion using homologous recombination. Homologous recombination permits site specific modifications in endogenous genes and thus inherited or acquired mutations may be corrected, and/or novel alterations may be engineered into the genome. Homologous recombination and site-directed integration in plants are discussed in, for example, U.S. Pat. Nos. 5,451,513; 5,501,967 and 5,527,695.

In some embodiments, minicells are produced by deleting, mutating, knocking out, or disrupting minC, minD, and/or minC and minD gene(s) in bacteria by traditional gene engineering techniques including homologous recombination. In other embodiments, minicells are produced by overexpressing certain genes such as ftsZ and/or minE in bacteria.

In some embodiments, the present disclosure teaches mutating cell populations by introducing, deleting, or replacing selected portions of genomic DNA. Thus, in some embodiments, the present disclosure teaches methods for targeting mutations to a specific locus such as ftsZ, minC, minD, minC/D, and minE. In other embodiments, the present disclosure teaches the use of gene editing technologies such as ZFNs, TALENS, CRISPR/Cas system or homing endonucleases, to selectively edit target DNA regions. In aspects, the targeted DNA regions is ftsZ, minC, minD, minC/D, and minE.

Engineered nucleases such as zinc finger nucleases (ZFNs), Transcription Activator Like Effector Nucleases (TALENs), engineered homing endonucleases, and RNA or DNA guided endonucleases, such as CRISPR/Cas such as Cas9 or CPF1, are particularly appropriate to carry out some of the methods of the present disclosure. Additionally or alternatively, RNA targeting systems can use used, such as CRISPR/Cas systems have RNA targeting nucleases.

In some embodiments, one skilled in the art can appreciate that the Cas9 disclosed herein can be any variant described in the literature, including but not limited to the functional mutations described in: Fonfara et al. Nucleic Acids Res. 2014 Feb;42(4):2577-90; Nishimasu H. et al. Cell. 2014 Feb 27; 156(5):935-49; JinekM. et al. Science. 2012 337:816-21; and JinekM. et al. Science. 2014 Mar 14; 343(6176); see also U.S. Pat. App. No. 13/842,859 filed Mar. 15, 2013, which is hereby incorporated by reference; further, see U.S. Pat. Nos. 8,697,359; 8,771,945; 8,795,965; 8,865,406; 8,871,445; 8,889,356; 8,895,308; 8,906,616; 8,932,814; 8,945,839; 8,993,233; and 8,999,641, which are all hereby incorporated by reference. Thus, in some embodiments, the systems and methods disclosed herein can be used with the wild type Cas9 protein having double-stranded nuclease activity, Cas9 mutants that act as single stranded nickases, deactivated Cas9 (dCas9) that has no nuclease activity, or other mutants with modified nuclease activity.

In some examples, a Type II nuclease may be catalytically dead (e.g. dCas9, “dead Cas9,” “deactivated Cas9”) such that it binds to a target sequence, but does not cleave. dCAS9 is a variant of the CAS9 protein (CRISPR) that has had its active site altered to no longer be able to edit genomes, but can still bind to highly specific segments of the genome using a guide RNA. This protein can stop transcription of the gene if bound. In some embodiments, the dCAS9 gene can be placed under inducible control so that its expression would be controlled. The guide RNA corresponding to the knockout within the Min system could be included on a plasmid or cut into the genome and placed under inducible control. Upon induction with this system, the guide RNA would direct the dCAS9 protein to the gene within the Min system in order to stop its expression. The stopping of expression of this gene such as minC, minD, and minC/D would result in the formation of minicells.

In some embodiments, the present disclosure teaches uses of the genetic manipulation technique using Lambda-Red recombination system in order to edit genome integrated with exogenous, heterologous expression cassette such as an selectable marker such as antibiotic resistant gene. In some embodiments, an selectable marker such as antibiotic resistant gene is integrated into the host genome (e.g. bacteria) in order to knockout minC/D/CD gene for inducing minicell production. If the marker with antibiotic resistance is no longer desired after successfully selecting the minicells in which the target gene (such as minC/D/CD) is knocked out, the flippase can be used to remove the integrated antibiotic resistant gene cassette from the host genome. A fragment of linear DNA is inserted into the genome directed by that fragment homology to the genome. This can be used to knock in genes of interest or to knockout genes of interest by replacing them with an antibiotic resistance cassette such as Chloramphenicol-resistant gene, kanamycin-resistant gene, spectinomycin-resistant gene, streptomycin-resistant gene, ampicillin-resistant gene, tetracycline-resistant gene, erythromycin-resistant gene, bleomycin-resistant gene, and bleomycin-resistant gene. A successful genetic manipulation is then selected for using this antibiotic resistance cassette. If a flippase recombination target (FRT) site is included within the resistance cassette for further genetic manipulations, it can be used for removing the antibiotic resistant gene integrated into the genome in vivo after selection of target minicells. The enzyme used for this is recombinase flippase and is often expressed from a plasmid that can be removed from the cell line using a temperature sensitive origin of replication. Recombinase flippase recognizes two identical FRT sites on both the 5′ and 3′ ends of the antibiotic resistance cassette and removes the DNA between the two sites. In some embodiments, the FRT site can be included within an antibiotic resistance cassette to remove the antibiotic resistance cassette after its use.

The present disclosure teaches that rnc gene encoding ribonucleaseIII or genes encoding protease in E. coli, or its homolog or ortholog in other bacteria is deleted, mutated, knocked out, or disrupted by the genetic modification methods described above (such as gene engineering techniques including homologous recombination; gene editing technologies such as ZFNs, TALENS, CRISPR/Cas system or homing endonucleases; genetic engineering/manipulation technique using Lambda-Red recombination system).

In some embodiments, the minicell is derived from a bacterial cell having a mislocalized cell division. In other embodiments, the minicell is derived from a bacterial cell having a mislocalized cell division with deficient RNaseIII and/or protease activity.

### Strains for Minicell Production

In some embodiments, a E. coli P678-54 strain is obtained from Coli Genetic Stock Center (CGSC), and is used to produce minicells (Adler et al., 1967, Proc. Natl. Acad. Sci. USA 57:321-326; Inselburg J, 1970 J. Bacteriol. 102(3):642-647; Frazer 1975, Curr. Topics Microbiol. Immunol. 69:1-84).

In some embodiments, an anucleated cell is produced from a P678-54 E. coli parental strain. The anucleated cell produced from P678-54 parental bacterial strain is used as an anucleated cell-based platform and/or an agricultural formulation for the encapsulation and delivery of biologically active compounds such as inhibitory RNA molecules taught herein.

### Ribonuclease-Deficient Bacterial Strains

The present disclosure also provides the production of minicells from HT115 (DE3) using genetically-engineering techniques. HT115 (DE3) is a RNAi Feeding strain, which is an RNase III-deficient E. coli strain with IPTG-inducible T7 Polymerase activity. To induce dsRNA production from these plasmids, the HT115 bacteria is grown on special RNAi NGM feeding plates that contain IPTG and the ampicillin analog carbenicillin. Carbenicillin is preferred over ampicillin because it tends to be more stable. Accordingly, HT115 strain as a ribonuclease-deficient strains can be utilized to create ribonuclease-deficient and/or ribonuclease-free minicells. The DE3 designation means that respective strains contain the λDE3 lysogen that carries the gene for T7 RNA polymerase under control of the lacUV5 promoter. IPTG is required to maximally induce expression of the T7 RNA polymerase in order to express recombinant genes cloned downstream of a T7 promoter. HT115 (DE3) is suitable for expression from a T7 or T7-lac promoter or promoters recognized by the E.coli RNA polymerase: e.g. lac, tac, trc, ParaBAD, PrhaBAD and also the T5 promoter. The genotype of HT115 (DE3) is: F-, mcrA, mcrB, IN(rrnDrrnE)1, rnc14::Tn10(DE3 lysogen: lavUV5 promoter -T7 polymerase) (IPTG-inducible T7 polymerase) (RNAse III minus). This strain grows on LB or 2XYT plates. This strain is tetracycline resistant. Researchers using this strain can test for expression by transforming in one of the plasmids from the Fire Vector Kit (1999) (pLT76, e.g.) using standard CaCl2 transformation techniques. This strain is resistant to tetracycline, and can be cultivated at 37° C., LB, and aerobic. Researchers also use this strain to test the interference experiment of nematodes.

In some embodiments, ribonuclease-deficient minicells disclosed herein are produced from ribonuclease-deficient parental strains including, but are not limited to, HT115 (DE3). In other embodiments, HT115 (DE3) strain is genetically engineered by deleting, mutating, knocking out, or disrupting minC, minD, and/or minC and minD gene(s) to induce minicell production. In other embodiments, HT115 (DE3) strain is genetically engineered by overexpressing ftsZ and/or minE genes to induce minicell production.

In some embodiments, ribonuclease-deficient minicells disclosed herein can be produced from protease-deficient parental strains including, but are not limited to, BL21 (DE3), BL21-AI and LPS-modified BL21 (DE3), genetically engineered by deleting, mutating, knocking out, or disrupting gene(s) encoding ribonuclease III. In other embodiments, BL21 (DE3), BL21-AI and LPS-modified BL21 (DE3) strains, in which ribonuclease III expression is suppressed, disrupted and/or nullified, are further genetically engineered by deleting, mutating, knocking out, or disrupting minC, minD, and/or minC and minD gene(s) to induce minicell production. In other embodiments, BL21 (DE3), BL21-AI and LPS-modified BL21 (DE3) strains, in which ribonuclease III expression is suppressed, disrupted and/or nullified, are also genetically engineered by overexpressing ftsZ and/or minE genes to induce minicell production.

Minicells that have segregated from parent cells lack chromosomal and/or nuclear components, but retain the cytoplasm and its contents, including the cellular machinery required for protein expression. In some embodiments, minicells are ribonuclease-deficient because the parent cells are ribonuclease-deficient strains. Although chromosomes do not segregate into minicells, extrachromosomal and/or episomal genetic expression elements may segregate, or may be introduced into minicells after segregation from parent cells. In some embodiments, the disclosure is drawn to ribonuclease-deficient minicells comprising an expression element, which may be an inducible expression element. The inducible expression element such as an inducible promoter can be introduced to a recombinant plasmid used for homologous recombination to knock out and/or delete gene(s) involved to cell division and/or chromosomal partitioning such as minC, minD, and minC/D, a recombinant expression vector to overexpress gene(s) involved to cell division and/or chromosomal partitioning such as ftsZ and minE, and a recombinant expression vector for expressing an enzymatically active polypeptide including a protein of interest disclosed herein. In further embodiments, the inducible expression element comprises expression sequences operably linked to an open reading frame (ORF) that encodes proteins of interest disclosed herein. Optionally, at any point in the method, an inducing agent is provided in order to induce expression of an ORF that encodes proteins of interest disclosed herein.

In some embodiments, the disclosure teaches methods of making a ribonuclease-deficient bacterial minicell comprising at least one polynucleotide encoding an inhibitory RNA molecule that are directed to at least one target sequence in a pathogen. In some embodiment, the disclosure teaches method of preparing protease-deficient minicells from the host cells.. In some embodiment, the disclosure teaches method of preparing ribonuclease-deficient minicells from the host cells.

In further embodiments, an anucleated cell is produced from an eukaryotic cell. In further embodiments, the anucleated cell produced as described above is used as an anucleated cell-based platform and/or an agricultural formulation for the encapsulation and delivery of biologically active compounds such as inhibitory RNA molecules taught herein.

In some embodiments, minicells taught in the present disclosure is protease deficient or ribonuclease deficient. In some embodiments, said minicell is protease deficient. In some embodiments, said minicell is ribonuclease deficient. In some embodiments, said minicell is protease deficient and ribonuclease deficient. In some embodiments, said minicell is ribonuclease-deficient, and wherein said biologically active compound is a nucleic acid. In some embodiments, said biologically active compound is said nucleic acid is selected from the group consisting of an antisense nucleic acid, a single-stranded RNA, a double-stranded RNA, a hairpin RNA (hpRNA), a short-hairpin RNA (shRNA), a small-interfering RNA (siRNA), a microRNA (miRNA), a ribozyme, an aptamer, and combination thereof.

In some embodiments, the minicell taught herein is derived from a bacterial cell. In other embodiments, the minicell is derived from a bacterial cell having a mislocalized cell division. In further embodiments, the minicell is derived from a bacterial cell having a mislocalized cell division with deficient RNaseIII activity.

In some embodiments, said minicell is ribonuclease deficient. In some embodiments, minicells taught in the present disclosure is protease deficient or ribonuclease deficient.

### Protease-Deficient Bacterial Strains

The present disclosure also provides the production of minicells from B strains using genetically-engineering techniques including B strains including BL21, BL21 (DE3), and BL21-AI, all of which are deficient in Lon protease (cytoplasm) and OmpT protease (outer membrane). Accordingly, B strains as protease-deficient strains can be utilized to create protease-deficient and/or protease-deficient minicells. The DE3 designation means that respective strains contain the λDE3 lysogen that carries the gene for T7 RNA polymerase under control of the lacUV5 promoter. IPTG is required to maximally induce expression of the T7 RNA polymerase in order to express recombinant genes cloned downstream of a T7 promoter. BL21(DE3) is suitable for expression from a T7 or T7-lac promoter or promoters recognized by the E.coli RNA polymerase: e.g. lac, tac, trc, ParaBAD, PrhaBAD and also the T5 promoter. The genotype of BL21 (DE3) is: fhuA2 [lon] ompT gal (λ DE3) [dcm] ΔhsdS λ DE3 = λ sBamHIo ΔEcoRI-B int::(lacI::PlacUV5::T7 gene1) i21 Δnin5.

BL21-AIE. coli contains a chromosomal insertion of the gene encoding T7 RNA polymerase (RNAP) into the araB locus of the araBAD operon, placing regulation of T7 RNAP under the control of the arabinose-inducible araBAD promoter. Therefore, this strain is especially useful for the expression of genes that may be toxic to other BL21 strains where basal expression of T7 RNAP is leaky. The BL21-AI strain does not contain the Ion protease and is deficient in the outer membrane protease, OmpT. The genotype of BL21-AI is F⁻ompT hsdS_{B} (r_{B}⁻ m_{B}⁻) gal dcm araB::T7RNAP-tetA. The BL21-AI has an arabinose promoter that controls the production T7 RNA Polymerase, while the BL21 (DE3) has a lac promoter that controls the production of the T7 RNA Polymerase. This is significant because the lac promotion system is leaky. Therefore, the BL21-AI protein production is more tightly regulated due to the arabinose promotion system.

The present disclosure teaches that LPS (Lipopolysaccharide) modified BL21 (DE3) cells can be used. The LPS of the E. Coli is modified to be significantly less toxic. This LPS modified BL21 (DE3) cells if necessary. This could also be branched out to other gram-negative bacterial cells. Safe usage of gram-negative cells can be beneficial for anucleated cell-based platform and/or an industrial formulation.

ClearColi® BL21(DE3) cells are the commercially available competent cells with a modified LPS (Lipid IVA) that does not trigger the endotoxic response in diverse cells. For example, ClearColi cells lack outer membrane agonists for hTLR4/MD-2 activation; therefore, activation of hTLR4/MD-2 signaling by ClearColi® is several orders of magnitude lower as compared with E. coli wild-type cells. Heterologous proteins prepared from ClearColi® are virtually free of endotoxic activity. After minimal purification from ClearColi cells, proteins or plasmids (which may contain Lipid IVA) can be used in most applications without eliciting an endotoxic response in human cells. In ClearColi cells, two of the secondary acyl chains of the normally hexa-acylated LPS have been deleted, eliminating a key determinant of endotoxicity in eukaryotic cells. The six acyl chains of the LPS are the trigger which is recognized by the Toll-like receptor 4 (TLR4) in complex with myeloid differentiation factor 2 (MD-2), causing activation of NF-ƙB and production of proinflammatory cytokines. The deletion of the two secondary acyl chains results in lipid IVA, which does not induce the formation of the activated heterotetrameric TLR4/MD-2 complex and thus does not trigger the endotoxic response. In ClearColi® BL21(DE3) Electrocompetent Cells 4 MA145 Rev. 31OCT2016 addition, the oligosaccharide chain is deleted, making it easier to remove the resulting lipid IVA from any downstream product.

In some embodiments, protease-deficient minicells disclosed herein are produced from protease-deficient parental strains including, but are not limited to, BL21 (DE3), BL21-AI and LPS-modified BL21 (DE3). In other embodiments, BL21 (DE3), BL21-AI and LPS-modified BL21 (DE3) strains are genetically engineered by deleting, mutating, knocking out, or disrupting minC, minD, and/or minC and minD gene(s) to induce minicell production. In other embodiments, BL21 (DE3), BL21-AI and LPS-modified BL21 (DE3) strains are genetically engineered by overexpressing ftsZ and/or minE genes to induce minicell production.

In further embodiments, the present disclosure provides a new minicell-producing strain named as B8. This strain is the protease-deficient minicell-producing strain without the T7 RNA Polymerase. This minicell strain is produced from the BL21 (DE3) strain. While knocking out minC/D/CD, the T7 RNA Polymerase was silenced due to the homology of the introduced knockout via Lambda Red Transformation. This strain can be used for a need of a protease-deficient minicell, but not having the T7 RNA Polymerase. In some embodiments, minicells displayed an enzymatically active polypeptide such as complicated or toxic proteins on their surface, need to be more controlled and slower expression of the desired but complicated or toxic proteins.

Minicells that have segregated from parent cells lack chromosomal and/or nuclear components, but retain the cytoplasm and its contents, including the cellular machinery required for protein expression. In some embodiments, minicells are protease-deficient because the parent cells are protease-deficient strains. Although chromosomes do not segregate into minicells, extrachromosomal and/or episomal genetic expression elements may segregate, or may be introduced into minicells after segregation from parent cells. In some embodiments, the disclosure is drawn to protease-deficient minicells comprising an expression element, which may be an inducible expression element. The inducible expression element such as an inducible promoter can be introduced to a recombinant plasmid used for homologous recombination to knock out and/or delete gene(s) involved to cell division and/or chromosomal partitioning such as minC, minD, and minC/D, a recombinant expression vector to overexpress gene(s) involved to cell division and/or chromosomal partitioning such as ftsZ and minE, and a recombinant expression vector for expressing an enzymatically active polypeptide including a protein of interest disclosed herein. In further embodiments, the inducible expression element comprises expression sequences operably linked to an open reading frame (ORF) that encodes proteins of interest disclosed herein. Optionally, at any point in the method, an inducing agent is provided in order to induce expression of an ORF that encodes proteins of interest disclosed herein.

In some embodiments, the disclosure teaches methods of making a protease-deficient bacterial minicell comprising at least one polynucleotide encoding an inhibitory RNA molecule that are directed to at least one target sequence in a pathogen. In some embodiment, the disclosure teaches method of preparing protease-deficient minicells from the host cells.

In other embodiments, the present disclosure teaches production of protease-deficient minicells from B. subtilis strains such as CU403 DIVIVA, CU403,DIVIVB,SPO-, CU403,DIVIVB and CU403,DIVIVB 1 using by deleting, mutating, knocking out, or disrupting gene encoding WprA protease.

B. subtilis genetic manipulations work slightly differently than genetic manipulations in E. coli. B. subtilis is known to readily undergo homologous recombination if DNA containing homology to the existing genome is inserted. This is unlike E. coli; E. coli has mechanisms in place to degrade any non-natural linear DNA present. This difference can be utilized in order to knockout genes by designing an antibiotic resistance cassette flanked by homologous arms which correspond to the start and end of the gene that is desired to be knockout out.

The present disclosure provides the production of minicells from B. subtilis using genetically-engineering techniques. In some embodiments, B. subtilis strains including, but are not limited to CU403 DIVIVA (BGSC No. 1A196), CU403,DIVIVB,SPO- (BGSC No. 1A197), CU403,DIVIVB (BGSC No. 1A292), CU403,DIVIVB1 (BGSC No. 1A513), KO7 can be used as parental bacterial cells to produce minicells. B. subtilis strains are the commercially available and can be obtained from Bacillus Genetic Stock Center (BGSC). The catalog of strains is available on the document provided by publicly accessible BGSC webpage.

In some embodiments, Bacillus Subtilis stains including, but are not limited to CU403 DIVIVA, CU403,DIVIVB,SPO-, CU403,DIVIVB and CU403,DIVIVB1 can be genetically modified by knocking out gene encoding WprA Protease in these strains. WprA protease is known as one of the harshest proteases.

B. subtilis secretes no fewer than seven proteases during vegetative growth and stationary phase. Strains in which multiple protease genes have been inactivated have proved to be superior to wild type strains in production of foreign proteins. The KO7 is prototrophic, free of secreted proteases, and have marker-free deletions in PY79 genetic background. This KO7 is available from the BGSC as accession number 1A1133. KO7 Genotype: ΔnprE ΔaprE Δepr Δmpr ΔnprB Δvpr Δbpr.

In some embodiments, a seven-protease deletion strain, B. subtilis KO7, can be used for B. subtilis minicell production by knocking out DIV-IVA and DIV-IVB using genetic engineering techniques known in the art.

In some embodiments, an anucleated cell is produced from a P678-54 E. coli wild strain. In other embodiments, an anucleated cell is produced from a protease-deficient E. coli strain including BL21, BL21(DE3), BL21-AI, LPS-modified BL21 (DE3) and B8. In some embodiments, an anucleated cell is produced from a parental bacterial cell deficient in WprA protease. In some embodiments, an anucleated cell is produced from a protease deficient B. subtilis parental bacterial cell. In some embodiments, an anucleated cell is produced from produced from a protease deficient KO7 B. subtilis parental bacterial cell. In other embodiments, an anucleated cell is produced from a protease deficient B. subtilis parental bacterial cell selected from the group consisting of: (1) CU403,DIVIVA; (2) CU403,DIVIVB,SPO-; (3) CU403,DIVIVB; and (4) CU403,DIVIVB1, wherein at least one protease encoding gene has been repressed, deleted, or silenced. In further embodiments, an anucleated cell is produced from an eukaryotic cell. In further embodiments, the anucleated cell produced as described above is used as an anucleated cell-based platform and/or an agricultural formulation for the encapsulation and delivery of biologically active compounds such as inhibitory RNA molecules taught herein.

In some embodiments, the minicell taught herein is derived from a bacterial cell. In other embodiments, the minicell is derived from a bacterial cell having a mislocalized cell division. In further embodiments, the minicell is derived from a bacterial cell having a mislocalized cell division with protease activity.

In some embodiments, said minicell is protease deficient. In some embodiments, minicells taught in the present disclosure is protease deficient or ribonuclease deficient. In some embodiments, said minicell is protease deficient and ribonuclease deficient.

### Agriculturally Suitable Carrier

Compositions described herein can comprise an agriculturally suitable carrier. The composition useful for these embodiments may include at least one member selected from the group consisting of a tackifier, a microbial stabilizer, a fungicide, an antibacterial agent, a preservative, a stabilizer, a surfactant, an anti-complex agent, an herbicide, a nematicide, an insecticide, a plant growth regulator, a fertilizer, a rodenticide, a dessicant, a bactericide, a nutrient, or any combination thereof. In some examples, compositions may be shelf-stable. For example, any of the compositions described herein can include an agriculturally suitable carrier (e.g., one or more of a fertilizer such as a non-naturally occurring fertilizer, an adhesion agent such as a non-naturally occurring adhesion agent, and a pesticide such as a non-naturally occurring pesticide). A non-naturally occurring adhesion agent can be, for example, a polymer, copolymer, or synthetic wax. For example, any of the coated seeds, seedlings, or plants described herein can contain such an agriculturally suitable carrier in the seed coating. In any of the compositions or methods described herein, an agriculturally suitable carrier can be or can include a non-naturally occurring compound (e.g., a non-naturally occurring fertilizer, a non-naturally occurring adhesion agent such as a polymer, copolymer, or synthetic wax, or a non-naturally occurring pesticide).

In some embodiments, an anucleated cell-based platform described herein can be mixed with an agriculturally suitable carrier. The carrier can be a solid carrier or liquid carrier, and in various forms including microspheres, powders, emulsions and the like. The carrier may be any one or more of a number of carriers that confer a variety of properties, such as increased stability, wettability, or dispersability. Wetting agents such as natural or synthetic surfactants, which can be nonionic or ionic surfactants, or a combination thereof can be included in the composition. Water-in-oil emulsions can also be used to formulate a composition that includes the isolated bacteria (see, for example, U.S. Pat. No. 7,485,451). Suitable formulations that may be prepared include wettable powders, granules, gels, agar strips or pellets, thickeners, liquids such as aqueous flowables, aqueous suspensions, water-in-oil emulsions, etc. The formulation may include grain or legume products, for example, ground grain or beans, broth or flour derived from grain or beans, starch, sugar, or oil.

In some embodiments, the agricultural carrier may be soil or a plant growth medium. Other agricultural carriers that may be used include water, fertilizers, plant-based oils, humectants, or combinations thereof. Alternatively, the agricultural carrier may be a solid, such as diatomaceous earth, loam, silica, alginate, clay, bentonite, vermiculite, seed cases, other plant and animal products, or combinations, including granules, pellets, or suspensions. Mixtures of any of the aforementioned ingredients are also contemplated as carriers, such as but not limited to, pesta (flour and kaolin clay), agar or flour-based pellets in loam, sand, or clay, etc. Formulations may include food sources for the bacteria, such as barley, rice, or other biological materials such as seed, plant parts, sugar cane bagasse, hulls or stalks from grain processing, ground plant material or wood from building site refuse, sawdust or small fibers from recycling of paper, fabric, or wood.

Additional examples of agriculturally suitable carriers include dispersants (e.g., polyvinylpyrrolidone/vinyl acetate PVPIVA S-630), surfactants, binders, and filler agents.

Persons having skill in the art will appreciate that, unless otherwise noted, all references to an anucleated cell-based platform in the present disclosure can be read as referring to an agricultural formulation. Therefore, embodiments described in the present disclosure which refer to an anucleated cell-based platform will also be understood to refer to an agricultural formulation.

In some embodiments, provided is a biocontrol composition comprising a minicell comprising at least one polynucleotide encoding an inhibitory RNA molecule that are directed to at least one target sequence in a pathogen. In one embodiments, the minicell is applied with at least one agriculturally suitable carrier. In one embodiments, the carrier is a solid, liquid, emulsion or powder form. In one embodiments, the carrier increases stability, wettability, or dispersability. In some embodiments, the composition is applied to a subject by spraying (including use of drone or plane), injecting, soaking, brushing, dressing, dripping, or coating in a solution.

### Biocontrols

The present disclosure teaches the biologically active compounds as a biocontrol such as an inhibitory RNA molecule (interchangeably used as “RNAi molecule”)using RNA interference (RNAi). RNAi related nucleic acids, RNAi biomolecule, including an antisense, ssRNA, dsRNA, hpRNA, shRNA, miRNA, siRNA, and miRNA. These inhibitory RNA molecules can be achieved via internal production from a recombinant construct within the minicells or via external production and being loaded into the minicells. The RNAi molecules are applied for i) biotic stress by controlling insects, weeds, fungi, viruses, or parasites by targeted delivery of RNAi molecules to a target transcript within a target cell and release over time. In some embodiments, a biologically active compound as a biocontrol is a nucleic acid that is selected from the group consisting of an antisense nucleic acid, a single-stranded RNA (ssRNA), a double-stranded RNA (dsRNA), a hairpin RNA (hpRNA), a short-hairpin RNA (shRNA), a small-interfering RNA (siRNA), a microRNA (miRNA), a ribozyme, an aptamer, and combination thereof.

As used herein, RNA interference (RNAi) is a biological mechanism which leads to post transcriptional gene silencing (PTGS) triggered by double-stranded RNA (dsRNA) molecules to prevent the expression of specific genes. For example, in one embodiment, RNA interference may be accomplished as short hairpin RNA molecules may be imported directly into the cytoplasm, anneal together to form a dsRNA, and then cleaved to short fragments by the Dicer enzyme. This enzyme Dicer may processes the dsRNA into ^{~}21-22-nucleotide fragment with a 2-nucleotide overhang at the 3′ end, small interfering RNAs (siRNAs). The antisense strand of siRNA become specific to endonuclease-protein complex, RNA-induced silencing complex (RISC), which then targets the homologous RNA and degrades it at specific site that results in the knock-down of protein expression.

The present disclosure teaches a biocontrol composition comprising: a minicell comprising at least one polynucleotide encoding an inhibitory RNA molecule that are directed to at least one target sequence in a pathogen. In some embodiments, the biocontrol composition is a minicell encapsulating at least one polynucleotide encoding an inhibitory RNA molecule that are directed to at least one target sequence in a pathogen. In other embodiments, the biocontrol composition is at least one polynucleotide encoding an inhibitory RNA molecule encapsulated or protected by a minicell for RNAi against a pest or a pathogen. In further embodiments, the biocontrol composition is a minicell-encapsulated dsRNA (ME-dsRNA) taught herein.

In some embodiments, the inhibitory RNA molecule causes downregulation of said at least one target sequence in the pathogen. In other embodiments, the pathogen or a disease caused by the pathogen is suppressed upon application of the composition compared to a control pathogen lacking the application of the composition.

In some embodiments, a minicell platform disclosed herein can encapsulate biologically active compounds as biocontrols (e.g. RNAi molecule) and deliver them in a scalable, targeted, cost-effective manner.

A polynucleotide active agent may comprise one or more of an oligonucleotide, an antisense nucleic acid, a dsRNA, a ssRNA, a siRNA, a miRNA, a hpRNA, a shRNA, an enzymatic RNA, a recombinant DNA construct, an expression vector, and mixtures thereof. The minicell delivery system of the present disclosure may be useful for in vivo or in vitro delivery of the polynucleotide active agent.

The present disclose teaches a composition comprising: a minicell comprising a biocontrol agent. The biocontrol agent is encapsulated or encompassed by the minicell. The biocontrol agent is a biologically active agent. In some embodiments, the biocontrol agent is a nucleic acid encoding an inhibitory RNA molecule, which is an antisense RNA, a single-stranded RNA (ssRNA), a double-stranded RNA (dsRNA), a hairpin RNA (hpRNA), a short-hairpin RNA (shRNA), a small-interfering RNA (siRNA), a microRNA (miRNA), or combination thereof.

In some embodiments, the inhibitory RNA molecule is exogenously prepared and encapsulated by the minicell.

In some embodiments, the inhibitory RNA molecule is transcribed from at least one heterologous expression construct of a recombinant vector comprising a promoter operably linked to the at least one polynucleotide in a parental cell transformed with the recombinant vector, and the inhibitory RNA molecule is present within the minicell.

In some embodiments, the present disclosure teaches topical applications of RNA interference (RNAi)-based biocontrols (e.g. biofungicides) that target fungal genes through double-strand RNAs (dsRNAs). In some embodiments, the RNAi-based biocontrol is encapsulated and/or protected by a minicell taught herein, which could represent an effective, safe and species-specific biofungicidal alternative. In some embodiments, Escherichia coli derived anucleated minicells can be utilized as a cost-effective, scalable platform for dsRNA production and encapsulation. In other embodiments, minicell-encapsulated dsRNA (ME-dsRNA) is shielded from RNase degradation and stabilized even when exposed to outward environmental conditions (such as a field where a crop or fruit is easily infected by plant pathogens). The ME-dsRNA enables the persistence of dsRNA in field-like conditions. In further embodiments, ME-dsRNAs directed to target genes of a plant pathogen selectively can knock down the target genes and lead to significant fungal growth inhibition.

In one aspect, ME-dsRNAs in the present disclosure can be applied to one or more of the following non-limiting group of plant viruses, including pathogen gene targets, generally referred to as gene targets, or essential genes, which would be recognized and available to those of ordinary skill in the art without undue experimentation: member species of the genera Becurtovirus, Begomovirus, Curtovirus, Eragrovirus, Mastrevirus, Topocuvirus or Turncurtovirus, as well as Beet Curly Top Iran virus, Spinach Severe Curly Top Virus, Bean Golden Mosaic Virus, Beet Curly Top Virus, Eragrostis curvula Streak Virus, Maize Streak Virus, Tomato Pseudo-Curly Top Virus, Turnip Curly Top Virus, Tobacco Mosaic Virus, Tomato Spotted-Wilt Virus, Tomato Yellow Leaf Curl Virus, Cucumber Mosaic Virus, Potato Virus Y, Cauliflower Mosaic Virus, African Cassava Mosaic Virus, Plum Pox Virus, Brome Mosaic Virus, Potato Virus X, Citrus Tristeza Virus, Barley Yellow Dwarf Virus, Potato Leafroll Virus and Tomato and Bushy Stunt Virus.

In another aspect, ME-dsRNAs in the present disclosure can be applied to one or more of the following non-limiting group of plant fungal pathogens, including pathogen gene targets, generally referred to as gene targets, or essential genes, which would be recognized and available to those of ordinary skill in the art without undue experimentation: Albugo, Alternaria; Aphanomyces; Aspergillus; Ascochyta; Blumeria; Botrytis; Cercospora; Colletotrichum; Diplodia; Erysiphe; Fusarium; Gaeumannomyces; Helminthosporium; Leptosphaeria: Macrophomina; Magnaporthe; Melampsora; Mycospharella; Nectria; Penecillium; Peronospora; Phakopsora; Phoma; Phymatotrichum; Phytophthora; Plasmopara; Plasmodiophora; Podosphaera; Puccinia; Pythium; Pyrenophora; Pyricularia; Rhizoctonia; Scerotium; Sclerotinia; Septoria; Stemphylium; Thielaviopsis; Uncinula; Uromyces; Ustilago; Venturia; and Verticillium.

In one aspect, ME-dsRNAs in the present disclosure can be applied to one or more of the following non-limiting group of The biocontrol composition according to claim 13, wherein the pathogens: Alternaria alternata, Botrytis cinerea, Fusarium solani, Peronospora berteroae, Phakospora pachyrhizi, Penicillium expansum, Phialophora gregata, Phytophthora sojae, Sclerotinia sclerotiorum or Verticillium dahliae.

In some embodiments, resistance to fungal infections can be obtained in different plant species that receive a nucleic acid (i.e. dsRNA targeting a gene or genes associated with survival or growth of fungi causing the aforementioned fungal infections) via the minicell platform taught herewith.

In some embodiments, a minicell-mediated delivery of inhibitory RNA molecules targeting pathogen coding RNAs for degradation, resulting in the reduction of the encoded protein levels. In some embodiments, the minicell-encapsulated inhibitory RNA molecules (i.e. ME-dsRNAs) in the present disclosure target pathogen RNAs encoding proteins including but are not limited to Argonaute family proteins, chitin synthases, dicer-like proteins, proteins encoded by resistance genes, ABC family proteins, proteins encoded by meiotic silencing genes, sex-induced silencing proteins, proteins composed of the RISC Complex, proteins and/or receptors associated with development of pesticide resistance in a pest, and combination thereof.

In some embodiments, the target sequence is a Chitin Synthase 3a gene, a Chitin Synthase 3b gene, a Dicer-like 1 gene, a Dicer-like 2 gene or combination thereof. In some embodiments, ME-dsRNA targeting the target sequence taught herein can recognize a homolog or an ortholog or a paralog of the target sequence, having at least 80%, at least 85%, at least 90%, or at least 95% sequence identity.

In some embodiments, the biocontrol composition comprise more than one ME-dsRNA, which allows for suppression or degradation of more than one pathogen RNA target. In some embodiments, at least one additional polynucleotide encoding an inhibitory RNA molecule are directed to at least one different target sequence in a pathogen. In other embodiments, at least two polynucleotides encoding at least two different inhibitory RNA molecules are delivered to a subject by minicells.

In some embodiments, a parental bacterial cell producing a minicell can be transformed with at least two recombinant vectors each of which comprise a polynucleotide encoding a different inhibitory RNA molecule.

In other embodiments, a biocontrol composition comprises a minicell comprising at least two recombinant vectors each of which comprises a polynucleotide encoding a different inhibitory RNA molecule targeting a different pathogen RNA.

In further embodiments, a biocontrol composition comprises at least two minicells each of which comprises a different recombinant vector comprising a polynucleotide encoding a different inhibitory RNA molecule targeting a different pathogen RNA.

In some embodiments, the inhibitory RNA molecule targets at least one nucleic acid sequence encoding an amino acid sequence at least 80%, at least 81%, at least 82%, at least 83%, at least 84%, at least 85%, at least 86%, at least 87%, at least 88%, at least 89%, 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, at least 99.9%, or 100% identical to SEQ ID NO: 18, 20, 22 or 24.

In some embodiments, the inhibitory RNA molecule targets at least one nucleic acid sequence having at least 70%, at least 75%, at least 80%, at least 81%, at least 82%, at least 83%, at least 84%, at least 85%, at least 86%, at least 87%, at least 88%, at least 89%, 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, at least 99.9%, or 100% sequence identity to SEQ ID NO:1 or 17.

In some embodiments, the inhibitory RNA molecule targets at least one nucleic acid sequence having at least 70%, at least 75%, at least 80%, at least 81%, at least 82%, at least 83%, at least 84%, at least 85%, at least 86%, at least 87%, at least 88%, at least 89%, 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, at least 99.9%, or 100% sequence identity to SEQ ID NO:6 or 19.

In some embodiments, the inhibitory RNA molecule targets at least one nucleic acid sequence having at least 70%, at least 75%, at least 80%, at least 81%, at least 82%, at least 83%, at least 84%, at least 85%, at least 86%, at least 87%, at least 88%, at least 89%, 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, at least 99.9%, or 100% sequence identity to SEQ ID NO:9 or 21.

In some embodiments, the inhibitory RNA molecule targets at least one nucleic acid sequence having at least 70%, at least 75%, at least 80%, at least 81%, at least 82%, at least 83%, at least 84%, at least 85%, at least 86%, at least 87%, at least 88%, at least 89%, 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, at least 99.9%, or 100% sequence identity to SEQ ID NO: 13 or 23.

In some embodiments, a sense strand of the inhibitory RNA molecule forming dsRNA for RNAi is a nucleic acid sequence having at least 70%, at least 75%, at least 80%, at least 81%, at least 82%, at least 83%, at least 84%, at least 85%, at least 86%, at least 87%, at least 88%, at least 89%, 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, at least 99.9%, or 100% sequence identity to SEQ ID NO:4, 5, 7, 8, 12 or 16.

In other embodiments, an antisense strand of the inhibitory RNA molecule forming dsRNA for RNAi is a complementary nucleic acid sequence having at least 70%, at least 75%, at least 80%, at least 81%, at least 82%, at least 83%, at least 84%, at least 85%, at least 86%, at least 87%, at least 88%, at least 89%, 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, at least 99.9%, or 100% sequence identity to SEQ ID NO:4, 5, 7, 8, 12 or 16.

In some embodiments, a nucleic acid sequence used for producing inhibitory RNA molecule is taught herein, which share at least 70%, at least 75%, at least 80%, at least 81%, at least 82%, at least 83%, at least 84%, at least 85%, at least 86%, at least 87%, at least 88%, at least 89%, 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, at least 99.9%, or 100% sequence identity to SEQ ID NOs:2-5 or 7-8.

In some embodiments, a nucleic acid sequence used for producing inhibitory RNA molecule is taught herein, which share at least 70%, at least 75%, at least 80%, at least 81%, at least 82%, at least 83%, at least 84%, at least 85%, at least 86%, at least 87%, at least 88%, at least 89%, 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, at least 99.9%, or 100% sequence identity to SEQ ID NOs:10-12 or 14-16.

In some embodiments, a nucleic acid sequence used for producing inhibitory RNA molecule is taught herein, which share at least 70%, at least 75%, at least 80%, at least 81%, at least 82%, at least 83%, at least 84%, at least 85%, at least 86%, at least 87%, at least 88%, at least 89%, 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, at least 99.9%, or 100% sequence identity to SEQ ID NOs:4-5, 7-8, 12 or 16.

Methods of alignment of sequences for comparison are well known in the art. Various programs and alignment algorithms are described in: Smith and Waterman (Adv. Appl. Math., 2:482, 1981); Needleman and Wunsch (J. Mol. Biol., 48:443, 1970); Pearson and Lipman (Proc. Natl. Acad. Sci., 85:2444, 1988); Higgins and Sharp (Gene, 73:237 44, 1988); Higgins and Sharp (CABIOS, 5:151 53, 1989); Corpet et al. (Nuc. Acids Res., 16:10881 90, 1988); Huang et al. (Comp. Appls Biosci., 8:155 65, 1992); and Pearson et al. (Meth. Mol. Biol., 24:307 31, 1994). Altschul et al. (Nature Genet., 6:119 29, 1994) presents a detailed consideration of sequence alignment methods and homology calculations.

In the case of pathogens, the target or essential gene may, for example, be a house-keeping or other gene, which is essential for viability or proliferation of the pathogen. The attenuation or silencing of the target gene may have various effects (also depending on the nature of the target gene). Silencing or attenuating said target gene results in loss or reduction of the pathogen’s harmful effects, i.e., pathogenicity in pathogens, or gain or increase of an agronomic trait in plants. Said agronomic trait can be selected from the group consisting of disease resistance, herbicide resistance, resistance against biotic or abiotic stress, and improved nutritional value. In this context, the target gene may, for example, be selected from the group consisting of genes involved in the synthesis and/or degradation of proteins, peptides, fatty acids, lipids, waxes, oils, starches, sugars, carbohydrates, flavors, odors, toxins, carotenoids, hormones, polymers, flavinoids, storage proteins, phenolic acids, alkaloids, lignins, tannins, celluloses, glycoproteins, and glycolipids. All these sequences are well known to the person skilled in the art and can be easily obtained from DNA data bases by those of ordinary skill in the art (e.g., GenBank).

The present disclosure teaches the advantage of the minicell platform, which is that the encapsulation capsule and biomolecule of interest, in this case dsRNA, can both be produced in one fermentation batch. Once the dsRNA is produced and encapsulated in the minicell, the dsRNA is significantly more stable than dsRNA on its own. In some embodiments, the minicell platform has proven to significantly enhance the stability of dsRNA.

The present disclosure provides the development and applicability of minicell-based RNAi technology, as a nontoxic alternative to chemical fungicides. The disclosure presents a robust, scalable platform for producing Minicell-encapsulated-dsRNAs (ME-dsRNAs) using a prokaryotic expression system that sufficiently addresses the major shortcomings of exogenous dsRNA delivery; especially those related to stability, efficacy and scalability. Using this platform, inventors were able to produce a minicell-encapsulated dsRNA delivery system, which demonstrated efficacy and specificity of ME-dsRNAs in inhibiting fungal growth. This synthetic biology platform has the potential to be incorporated into commercial disease management programs against B. cinerea and other economically-important phytopathogenic fungi.

The present disclosure teaches the use of minicell platform for RNAi technology in integrated pest/disease management programs for controlling pests, viruses, and other fungal pathogens, in a sustainable way.

In some embodiments, bacteria-derived minicells can be utilized as a cost-effective, scalable platform for dsRNA production and encapsulation.

For example, minicell-encapsulated dsRNA (ME-dsRNA) is shielded from RNase degradation and stabilized, enabling the persistence of dsRNA to a subject for a long-term period at least 1 hour, at least 12 hours, at least 1 day, at least 2 days, at least 3 days, at least 4 days, at least 5 days, at least one week or at least two weeks after application of the ME-dsRNA. The present disclosure teaches that ME-dsRNAs targeting chitin synthase class III (Chs3a, Chs3b) and DICER-like proteins (DCL1 and DCL2) genes of B. cinerea selectively knocked-down the target genes and led to significant fungal growth inhibition.

In some embodiments, the potential of ME-dsRNAs to enable the commercial application of RNAi based species-specific biocontrol agents that are comparable in efficacy to conventional synthetics. ME-dsRNAs offer a platform that can readily be translated to large-scale production and deployed to control pests.

The exogenous application of dsRNA or siRNA to reduce B. cinerea infection has already been reported in many pathobiological systems (Wang et al 2016; Mcloughlin et al 2018). However, due to limited field stability and high production cost, sprayable dsRNA biofungicides are not yet practically feasible at a large scale. Instead, the present disclosure provide solutions to the applicability issues associated with sprayable dsRNAs by teaching a dsRNA bioproduction platform that is based on bacterial minicell carrier systems.

As described in Example 4, it was proven in greenhouse trials in which naked-dsRNA and minicells with encapsulated dsRNA were applied 5 days prior to inoculation of B. cinerea, respectively. Minicells with dsRNA provide full coverage for 5 days after inoculation, showing protection for a full 10 days, while the dsRNA on its own fails. The minicell platform has proven to significantly enhance the stability of dsRNA.

In some embodiments, the suppressive or inhibitory effects of ME-dsRNAs on growth, survival, development, and/or reproduction of a plant pathogen and/or pest can last at least 1 hour, at least 12 hours, at least 1 day, at least 2 days, at least 3 days, at least 4 days, at least 5 days, at least one week or at least two weeks after treatment, application, administration, or introduction of ME-dsRNAs onto a subject.

The present disclosure provides the development and applicability of minicell-based RNAi technology in agriculture, as a nontoxic alternative to chemical fungicides. The disclosure presents a robust, scalable platform for producing ME-dsRNAs using a prokaryotic expression system that sufficiently addresses the major shortcomings of exogenous dsRNA-based biofungicides; especially those related to stability, efficacy and scalability. Using this platform, inventors were able to produce a 33 mg/liter minicell-encapsulated dsRNA delivery system, which demonstrated efficacy and specificity of ME-dsRNAs in inhibiting fungal growth under greenhouse conditions. This synthetic biology platform has the potential to be incorporated into commercial disease management programs against B. cinerea and other economically-important phytopathogenic fungi.

The present disclosure teaches the use of minicell-based RNAi technology in integrated pest/disease management programs for controlling pests, viruses, and other fungal pathogens, in a sustainable way that addresses public concerns around GMOs and overuse of synthetic chemicals.

In some embodiments, a compensatory relationship between DCL1 and DCL2 gene transcripts, where the silencing of one gene upregulated the expression of the other.

### Methods for Controlling a Pathogen

In some embodiments, the controlling of a pest or a pathogen refers to (i) the suppressing, inhibiting, limiting, or controlling the growth of or killing one or more a pest or a pathogen that infects a plant, a crop, a vegetable, a herb, a fruit and the like and (ii) the preventing, treating or curing of a disease or condition in a plant suffering therefrom.

The present disclosure teaches the use, application, introduction or administration of ME-dsRNAs as a biocontrol agent or a biopesticide for suppression or degradation of target pathogen RNAs to control a pest or a pathogen. The topical application of ME-dsRNA is one way for the pathogen control by contacting the ME-dsRNA with a subject.

The term “contact with” or “uptake by” an organism (e.g., a plant or pest or herbivore), with regard to a nucleic acid molecule encapsulated by a minicell, includes internalization of the nucleic acid molecule (e.g. inhibitory RNA molecule) into the organism, for example and without limitation: ingestion of the molecule by the organism (e.g., by feeding); contacting the organism with a composition comprising the minicell comprising the nucleic acid molecule; soaking of organisms with a solution comprising the minicell comprising the nucleic acid molecule; injecting the organism with a composition comprising the minicell comprising the nucleic acid molecule; and spraying the organism with an aerosol composition comprising the minicell comprising the nucleic acid molecule.

The present disclosure provides a method for controlling a pathogen comprising the steps of: introducing into a minicell at least one polynucleotide encoding an inhibitory RNA molecule that are directed to at least one target sequence in a pathogen. In some embodiments, the minicell is applied, introduced, administered to a subject or contacted with a subject. In some embodiments, the inhibitory RNA molecule causes downregulation of said at least one target sequence in the pathogen, and the pathogen or a disease caused by the pathogen is suppressed upon application of the composition compared to a control pathogen lacking the application of the composition. In some embodiments, said minicell is applied with at least one agriculturally suitable carrier. In some embodiments, the carrier is a solid, liquid, emulsion or powder form. In some embodiments, the carrier increases stability, wettability, or dispersability. In some embodiments, the method further comprises the step of: introducing at least one additional polynucleotide encoding an inhibitory RNA molecule that are directed to at least one different target sequence in a pathogen. In some embodiments, the suppression lasts at least 1 hour, at least 12 hours, at least 1 day, at least 2 days, at least 3 days, at least 4 days, at least 5 days, at least one week or at least two weeks.

According to the method of the present disclosure, the minicell is derived from a bacterial cell having a bacterial cell having a mislocalized cell division with deficient RNaseIII activity.

According to the method of the present disclosure, the inhibitory RNA molecule is an antisense RNA, a single-stranded RNA (ssRNA), a double-stranded RNA (dsRNA), a short-hairpin RNA (shRNA), a small-interfering RNA (siRNA), a microRNA (miRNA), or combination thereof. In some embodiments, the inhibitory RNA molecule is exogenously prepared and encapsulated by the minicell. In some embodiments, the inhibitory RNA molecule is transcribed from at least one heterologous expression construct comprising a promoter operably linked to the at least one polynucleotide within the minicell.

According to the method of the present disclosure, the target sequence is selected from genes encoding Argonaute family proteins, chitin synthases, dicer-like proteins, proteins encoded by resistance genes, ABC family proteins, proteins encoded by meiotic silencing genes, sex-induced silencing proteins, proteins composed of the RISC Complex, and combination thereof. In some embodiments, the target sequence is a Chitin Synthase 3a gene, a Chitin Synthase 3b gene, a Dicer-like 1 gene, a Dicer-like 2 gene or combination thereof. In some embodiments, the inhibitory RNA molecule targets a nucleic acid sequence encoding an amino acid sequence at least 90% identical to SEQ ID NO: 18 or 20. In some embodiments, the inhibitory RNA molecule targets a nucleic acid sequence encoding an amino acid sequence at least 90% identical to SEQ ID NO:22 or 24.

According to the method of the present disclosure, the pathogen is selected from the group consisting of Albugo, Alternaria; Aphanomyces; Aspergillus; Ascochyta; Blumeria; Botrytis; Cercospora; Colletotrichum; Diplodia; Erysiphe; Fusarium; Gaeumannomyces; Helminthosporium; Leptosphaeria: Macrophomina; Magnaporthe; Melampsora; Mycospharella; Nectria; Penecillium; Peronospora; Phakopsora; Phialophora, Phoma; Phymatotrichum; Phytophthora; Plasmopara; Plasmodiophora; Podosphaera; Puccinia; Pythium; Pyrenophora; Pyricularia; Rhizoctonia; Scerotium; Sclerotinia; Septoria; Stemphylium; Thielaviopsis; Uncinula; Uromyces; Ustilago; Venturia; and Verticillium. In some embodiments, the pathogen is Botrytis cinerea, Fusarium solani, Peronospora berteroae, Phakospora pachyrhizi, Phialophora gregata, Phytophthora sojae, Sclerotinia sclerotiorum or Verticillium dahliae.

According to the method of the present disclosure, the subject is a plant selected from the group consisting of: corn, wheat, rice, barley, oat, rye, sorghum, millet, sugar cane, strawberry, blueberry, raspberry, blackberry, apple, grape, pear, peach, melon, cucumber, pumpkin, squash, soybean, sugar beet, spinach, swiss chard, potato, eggplant, tomato, sunflower, safflower, gladiolus, cotton, canola, alfalfa, cannabis, Brassica, peanut, tobacco, banana, duckweed, pineapple, date, onion, cashew, pistachio, citrus, rose, almond, coffee, bean, legume, watermelon, squash, cabbage, turnip, mustard, cacti, pecan, flax, sweet potato, coconut, avocado, cantaloupe, vegetables, and herbs.

According to the method of the present disclosure, the minicell is applied to the subject by spraying, injecting, soaking, brushing, dressing, dripping, or coating in a solution.

The present disclosure provides a method for preventing, treating or curing a disease or condition in a subject suffering therefrom a pathogen, comprising the steps of: introducing into a minicell at least one polynucleotide encoding an inhibitory RNA molecule that targets a nucleic acid sequence encoding an amino acid sequence at least 90% identical to SEQ ID NO:18, 20, 22 or 24, and applying the minicell to the subject. In some embodiments, the inhibitory RNA molecule causes downregulation of said at least one target sequence in the pathogen, and the pathogen or a disease caused by the pathogen is suppressed upon application of the composition compared to a control pathogen lacking the application of the composition.

### EXAMPLE

The following examples are given for the purpose of illustrating various embodiments of the disclosure and are not meant to limit the present disclosure in any fashion. Changes therein and other uses which are encompassed within the spirit of the disclosure, as defined by the scope of the claims, will occur to those skilled in the art.

### Example 1. Characterization, Purification, and Stability of Minicells Encapsulated-dsRNAs

### Minicell Production

Deletions of the E. coli cell cycle-related genes minCDE produce a large number of intact and stable minicells (MacDiarmid et al 2007; Hale et al 1983). To produce a large number of minicells with compromised (or deficient) RNase-III activity, inventors generated E. coli mutants in which minCDE and rnc are knocked out. These mutations were made using Lambda Red mediated recombination (Datsenko et al, 2000).

One E. coli minicells-producing bacterial colony (i.e. the E. coli mutants with minCDE and rnc knocked out) was picked from an agar plate and inoculated in a culture of Luria Broth (~450 mL) in a shaking incubator at 37° C. for overnight growth. During the overnight growth process, the bacterial cells produced anucleate minicells. The sample was then removed from the shaking incubator and poured into three 250 mL of centrifuge tubes; 150 mL of sample in each. The tubes containing the bacterial/minicells suspension was subjected to initial centrifugation at 2,000 x g to remove bacterial cells. The supernatant was transferred to three clean 250 mL of centrifuge tubes and centrifuged at 10,000 x g to form the pellet of the minicells. The sizing and purity were analyzed using Beckman-Coulter Multisizer. For scanning electron microscopic (SEM) observations, minCDE mutant bacterial cells and minicells samples were processed. The samples were first fixed with glutaraldehyde 2-2.5% for 1 h and washed three times with 0.2 M sodium cacodylate buffer (pH 7.4). The sample were then post-fixed in 1% osmium tetraoxide (OsO4) for 1h, dehydrated using increasing ethanol gradient from 30% to 100% for 10 min each and finally incubated with 50, 75, and 100% hexamethyldisilazane (HMDS) in ethanol 10 min each. All liquids were aspirated off to make sure surface is completely dry and the samples was gold-coated and observed under a scanning electron microscope at an operating voltage of 3 kV (Zeiss Sigma VP HD field emission SEM).

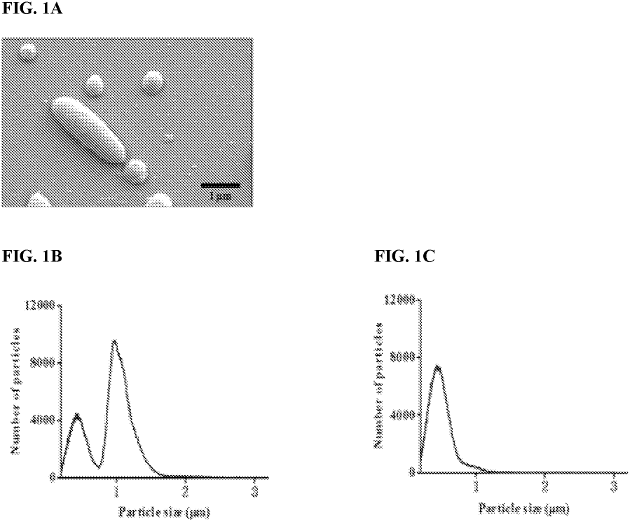

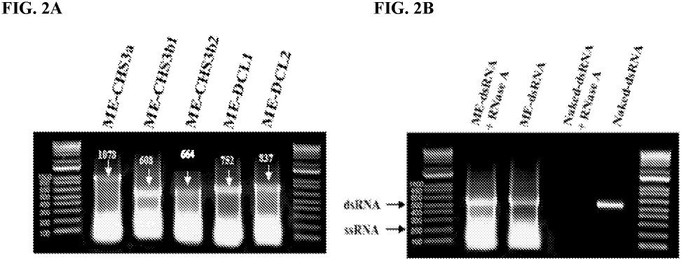

FIG. 1A shows the minicells derived from the E. coli mutants in which minCDE and rnc are knocked out, thus allowing the expression of dsRNAs as RNaseIII activity is suppressed due to the rnc mutation/knock-out.

### Construct Design and Expression of Minicells-Encapsulated dsRNAs

A T7 RNA polymerase/promoter system was used to express all dsRNAs constructs from a modified pGEX backbone. In the constructs, two promoters with T7 RNA polymerase binding sites were added to each side of the dsRNA expression cassette (comprising a promoter operably linked to a dsRNA target sequence) to produce a non-hairpin double stranded RNA that binds due to binding affinity between sense anti-sense strands of RNA. A convergent transcription from opposing promoters is used to induce RNAi-mediated gene inhibition. A convergent transcription using convergent promoters can be used in bacterial cells to invoke gene-specific silencing via RNAi .

The T7 RNA polymerase expression was controlled by the PBAD promoter and expressed from the genome of the minicell-producing E. coli. The T7 RNA polymerase was co-expressed with a novel dsRNA binding protein located on the pGEX construct to stabilize the RNA in vivo. All dsRNA targets were produced from the same foundational modified pGEX backbone, but with different targets located between the flanking T7 RNA polymerase/promoter systems. In vivo expression of dsRNAs was done in the BioFlo® 120 bioreactor system (Eppendorf, Enfield CT, USA). A seed culture was first grown from a colony on an agar plate in TB media selecting for the modified pGEX plasmid with ampicillin (100 µg/mL). After the overnight growth, the seed culture was inoculated into the bioreactor system and the desired dsRNA was expressed using the above promotion systems and encapsulated within the minicell. Inventors transformed this E. coli mutant with DNA constructs to express dsRNAs targeting B. cinerea genes that are essential for pathogenicity. The first group of genes inventors used are cell-wall integrity-related genes, including two isoforms of chitin synthase class III; Chs3a and Chs3b. Chitin is the rigid carbohydrate polymer that constitutes the cell wall of fungi. In B. cinerea, seven different classes of the chitin synthase genes have been identified, with duplications present in class III (Chs3a and Chs3b). Deletions of Chs3a significantly reduce the virulence and radial growth of B. cinerea (Soulie et al 2006).

ME-CHS3a is a minicell transformed with the expression construct producing dsRNA Chs3a target from the convergent transcription of a polynucleotide of SEQ ID NO:4 (BCC1; 1078 bp). ME-CHS3b 1 is a minicell transformed with the expression construct expressing dsRNA Chs3b target from the convergent transcription of a polynucleotide of SEQ ID NO:7 (BCC3; 608 bp). ME-CHS3b2 is a minicell transformed with the expression construct expressing dsRNA Chs3b target from the convergent transcription of a polynucleotide of SEQ ID NO:8 (BCC4; 664 bp). T7 promoter on each side of DNA sequence allows for expressing RNA sequence of interest in a convergent manner, resulting in non-hairpin dsRNA sequence with sense and antisense binding.

The second group of genes inventors targeted are the B. cinerea RNAi-machinery related genes, DCL1 and DCL2. DCL1 and DCL2 are involved in the synthesis of fungal siRNA-effectors to suppress plant immunity and facilitate gray mold disease progression (Wang et al 2016).

ME-DCL1 is a minicell transformed with an expression construct expressing dsRNA Dc11 target from the convergent transcription of a polynucleotide of SEQ ID NO:12 (752 bp). ME-DCL2 is a minicell transformed with the expression construct expressing dsRNA Dc12 target from the convergent transcription of a polynucleotide of SEQ ID NO: 16 (837 bp). T7 promoter on each side of DNA sequence allows for expressing RNA sequence of interest in a convergent manner, resulting in non-hairpin dsRNA sequence with sense and antisense binding.

The transformed E. coli mutants were cultured in batch phase, induced for dsRNA expression and processed via differential centrifugation, to recover purified minicells at the level of purity depicted in FIG. 1C, a single peak representing the purified minicells.

### Stability of Minicells-Encapsulated dsRNA

As a control, naked-dsRNAs were produced in vitro. The IVT-dsRNAs were synthesized using the same constructs as the in vivo production. A linear template for the IVT reaction was generated via a polymerase chain reaction (PCR) with the following primers 5′-TTCCTCAAGACGCGGAGATG-3′ and 5′-CAGACAAGCTGTGACCGTCT-3′. Phusion Polymerase® was used in this PCR reaction according to the manufacturers’ recommendation (New England Biolabs, Ipswich, MA, USA). The PCR reactions were cleaned up using the Monarch® DNA Cleanup Kit (New England Biolabs, Ipswich, MA, USA) according to the manufacturer’s recommendation. The entirety of the elution was visualized using agarose gel electrophoresis. The band was visualized at the expected product size and extracted using the Monarch® DNA Gel Extraction kit according to the manufacturer’s recommendations (New England Biolabs). The PCR template was used in the HiScribe™ T7 High Yield RNA Synthesis Kit according to the manufacturers’ protocol (New England Biolabs, Ipswich, MA, USA). The IVT reaction was cleaned up using the Direct-zol™ RNA MiniPrep Plus Kit (Zymo Research, Irvine CA, USA) according to a manufacturers’ protocol. The manufacturers’ protocol was followed with the addition of an RNase T1 digest (ThermoFisher Scientific, Waltham MA, USA) in conjunction with a DNase digest.

The total RNA extracts for the minicell produced dsRNA were obtained using the Direct-zol™ RNA MiniPrep Plus Kit (Zymo Research, Irvine CA, USA) according to the manufacturers’ protocol with the modification above. A portion of this total RNA extract was visualized using native agarose gel electrophoresis. The band that was visualized at the expected product size was extracted from the gel using the Zymoclean Gel RNA Recovery Kit (Zymo Research, Irvine CA, USA) according to the manufacturers’ protocol. The RNA extracted from the gel was quantified using the average from both a nanodrop (Biotek Take3™) and the Quant-iT™ RiboGreen™ RNA Assay Kit (ThermoFisher Scientific, Waltham MA, USA) according to the manufactures recommendation. The high range quantification protocol was used for the RiboGreen™ RNA Assay Kit (ThermoFisher Scientific, Waltham MA, USA) at a 200x in well dilution. The RNA remaining in the total RNA was quantified using the ratio of volumes between the elution volume and the volume visualized on the gel. The gel slice was eluted in the same volume as was run on the gel. The amount of product RNA from the entire minicell fraction was calculated from the ratio of volumes between the analysis fraction and the remaining minicell fraction. The in vitro produced dsRNA was quantified using the same procedure but the total RNA fraction was obtained from the IVT reaction cleanup detailed above.

The dsRNA from the pure minicell solution was extracted and size-verified. As shown in FIG. 2A, DsRNA Chs3a (1078 bp) is present in ME-CHS3a; DsRNA Chs3b1 (608 bp) is present in ME-CHS3b1; DsRNA Chs3b2 (664 bp) is present in ME-CHS3b2; DsRNA Dcl1 (752 bp) is present in ME-DCL1; and DsRNA Dcl2 (837 bp) is present in ME-DCL2.

The protection capacity of the ME-dsRNA from the RNase A degradation was examined using the ME-CHS3b1. The equal amount (2.8 µg) of ME- and naked-dsRNA was treated with 100 µg of RNase A (ThermoFisher Scientific, Waltham MA, USA) for 30 minutes at 37° C. The RNase A treated ME-encapsulated dsRNA was extracted as describe previously. Nuclease treated and untreated ME- or naked-dsRNA were then resolved on agarose gel.

The ability of minicells to protect dsRNAs from RNase A degradation was confirmed in vitro (FIG. 2B). Compared to naked-dsRNAs, minicells were resistant to RNase A treatment and yielded an intact dsRNA band on the agarose gel, as shown in FIG. 2B. ME-dsRNA remained unaffected by RNase A treatment, with both treated and untreated conditions producing a band of the same relative intensity, visualized with native agarose gel electrophoresis. These results indicate that the RNase cannot access dsRNA that is minicell-encapsulated. The naked-dsRNA was degraded beyond the limit of detection given the same RNase A treatment, while the non-treated naked-dsRNA band was visualized using the same method indicating that the RNase A was responsible for the degradation.

### Example 2. Effects of the Minicells Encapsulated dsRNAs (ME-dsRNAs) as a Biofungicide on B. Cinerea

The potential of ME-dsRNAs to function as an effective biofungicide was examined in vitro by exposing actively growing fungal mycelia of B. cinerea to ME-dsRNAs. In vitro mycelial growth inhibition assay was performed as follows.

To study the effects of minicells-encapsulated dsRNA (ME-dsRNA) targeting various genes of Botrytis cinerea on mycelial growth, different concentrations of ME-dsRNA for the single (500, 250 and 125 ng/mL) and combined applications (1000, 500, and 250 ng/mL), respectively, were used. The mycelial growth inhibition assay was performed in 24-wells plate in which 1 ml of the potato dextrose broth (PDB) containing different concentrations of ME-dsRNA was added in each well. The mycelial plug (4 mm) from the leading edge of 5d-old potato dextrose agar (PDA) plate culture was cut using cork-borer and placed in each well. The diameter of the fungal growth was measured after 72 h after incubation at 25° C. Mycelial growth-inhibitory rates (IR, %), were calculated as IR = (1-x/y) × 100, where x and y were the diameter of fungal mycelium in the ME-dsRNA treated and empty-minicells-treated (control) samples, respectively. Three independent trials with three biological replicates in each trial were conducted for each treatment.

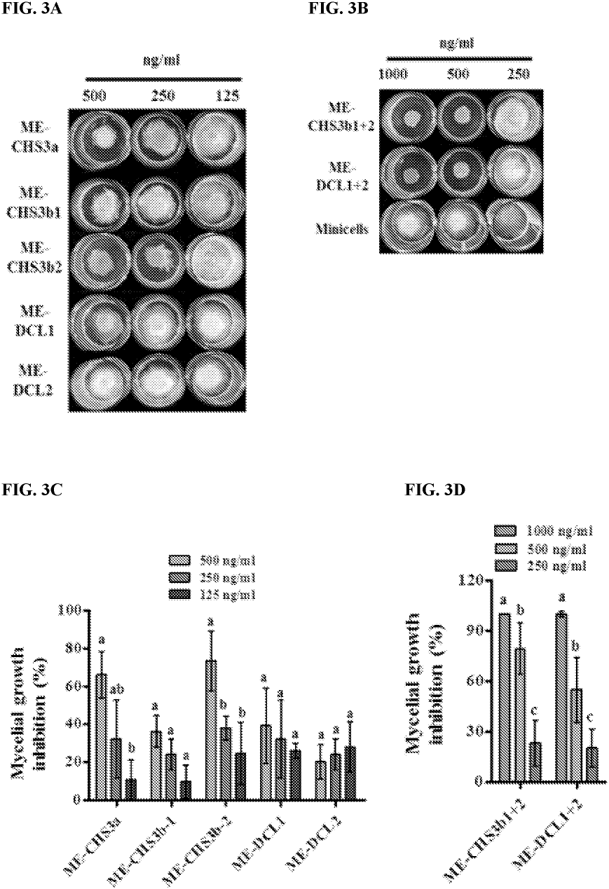

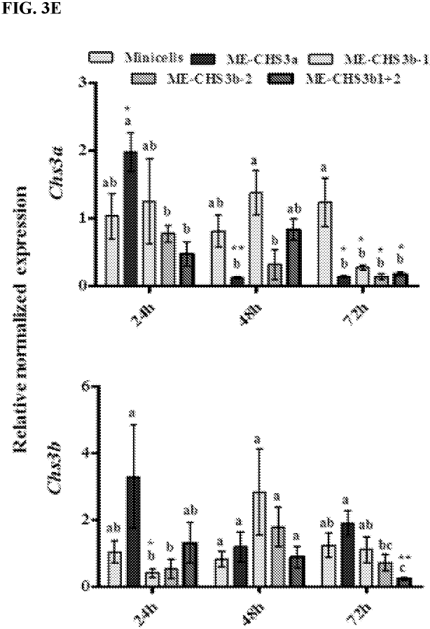

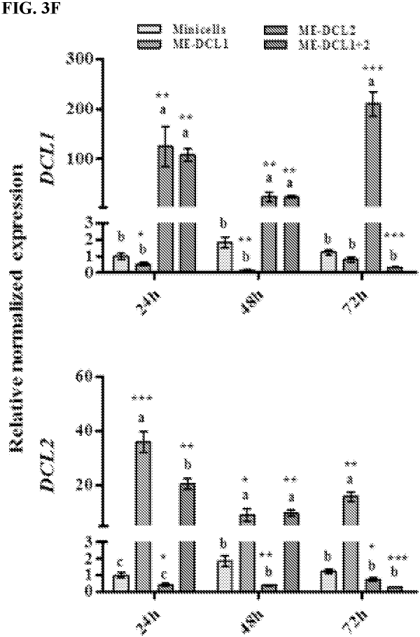

As shown in FIGS. 3A and 3C, both 500 ng/mL of ME-CHS3a and ME-CHS3b2 showed significant antifungal activity, and the combination of the two constructs targeting Chs3b (CHS3b1+2 at 1000 ng/mL) completely inhibited the mycelial growth (FIGS. 3B and 3D).

To further validate these findings at the molecular level, the relative expression of target genes was evaluated by qRT-PCR, using B. cinerea β-actin and Tubulin genes as housekeeping genes and an empty-minicell treatment (without dsRNAs present) as a negative control.

For the quantification of relative transcript abundance following ME-dsRNA treatments in vitro, a 4 mm mycelial plug was taken from the outer margin of freshly cultured (5-days-old) and placed in each well of the 24-wells plate containing 1 mL of PDB. ME-dsRNAs was applied at a dose of 1000 ng/mL for the combined treatments of ME-CHS3b1+2 and ME-DCL1+2, and 500 ng/mL for the single ME-dsRNA treatment. The mycelial tissues were then collected at 24, 48, and 72 h after treatment, frozen in liquid nitrogen and stored in -80° C. until RNA extraction. Total RNA from treated and control samples were extracted using a CTAB method with some minor modifications³². Briefly, the frozen fungal tissue were ground using Geno-grinder in 4 ml vials and 1.2 mL of CTAB extraction buffer [2% (w/v) CTAB (cetyl trimethyl ammonium bromide), 2% (w/v) PVP (polyvinylpyrrolidone, mol wt 40,000), 100 mM Tris-HCl (pH 8.0), 25 mM EDTA, 2 M NaCl, 0.05% spermidine trihydrochloride, and 2% of β -mercaptoethanol] was added to each sample in 2 mL centrifuge tubes. Tubes were vortexed vigorously for 2 min and then incubated at 65° C. for 30 min. Samples were vortexed every 10 min during incubation. After incubation, samples were transferred to 2 mL Eppendorf tube and centrifugated at 13200 rpm for 15 min at room temperature. The supernatant was transferred to another 2ml Eppendorf tube and an equal volume of chloroform-isoamyl alcohol (24:1) (Sigma-Aldrich Co., St Louis, USA) was added to each tube followed by vigorous vortexing for 2 min. Samples were then centrifuged at 13200 rpm for 15 min at room temperature and this step was repeated one more time. The aqueous supernatant was transferred to an Eppendorf tube and ¼ volume of 10 M LiCl (Sigma) was added to the supernatant. Tubes were mixed well by inverting them several times and then incubated for 2 h at -20° C. The total RNA was pelleted by centrifugation at 14000 rpm for 30 min at 4° C. The supernatant was discarded and the pellet was washed with 0.5 mL of 80% ethanol and centrifuged again at 14000 rpm for 5 min at 4° C. The supernatant was removed and the pellet was dried at room temperature. The RNA pellet was dissolved in RNase free water and the RNA quantity was estimated using a Synergy H1 hybrid reader (BioTek, Oakville, ON, Canada). All RNA samples were treated with DNase and then purified using EZ RNA Clean-Up Plus DNase Kit (EZ BioResearch, St Louis, Missouri, USA). The cDNA was synthesized from 1 µg of DNase-treated RNA using a cDNA synthesis kit (Applied biosystems, Foster City, USA) and according to the manufacturer’s instructions. Quantitative real time-PCR (qRT-PCR) was performed using CFX connect real-time detection System (Bio-Rad, Mississauga, ON), SsoFast™ EvaGreenR Supermix (Bio-Rad) and gene-specific primers. The gene expression of target genes was first normalized to the expression of two house-keeping genes from B. cinerea β-actin and Tublin). The accumulation of gene transcripts in each sample was calculated according to the 2^{-ΔΔ}Cq method and relative to the expression in the minicells-treated sample at 24 h. All the qRT-PCR reactions were performed in three biological and three technical replicates and results were analyzed using the gene study feature of the CFX manager software (Bio-Rad).

Treatments by ME-CHS3a and the combination of ME-CHS3b 1+2 significantly reduced the relative transcript level of Chs3a and Chs3b in fungal mycelia, respectively, at 72 hours post treatment (hpt), as shown in FIG. 3E.

Additionally, the reduction in Chs3a observed in response to ME-CHS3b2 and ME-CHS3b1+2 at 72 hpt (FIG. 3E), which could be explained by the high degree of similarities (60.5%) between Chs3a and Chs3b gene homologs (Choquer et al 2004). Interestingly, it has previously been reported that Chs3b transcripts were not detected in the mycelium of B. cinerea using the semi-quantitative RT-PCR approach (Choquer et al 2003). Likewise, inventors observed significant reduction in Chs3b transcript levels in responses to ME-CHS3b1+2 treatment, however, the level of the Chs3b transcripts was generally low as indicated by the high Cq values (i.e. 35 out of 40 PCR cycles). These datasets together suggest that ME-dsRNA targeting the Chs3b gene of B. cinerea suppressed both Chs3a and Chs3b, and the simultaneous suppression of both genes of chitin synthase class III might be crucial for the complete inhibition of B. cinereal mycelial growth.

Furthermore, the combined application of ME-dsRNA targeting DCL11 and DCL2 at 1000 ng/mL inhibited fungal growth at 72 hpt (FIGS. 3B and 3D). However, the treatment of ME-dsRNA individually targeting DCL1 (ME-DCL1) or DCL2 (ME-DCL2) did not lead to any significant mycelial growth inhibition (FIGS. 3A and 3C). Mutations in DCL1 or DCL2 did not compromise the virulence or growth of B. cinerea, whereas the fungus double mutant dcl1+2 showed reduced virulence. Results presented herewith support that silencing of both DCL1 and DCL2 is required to inhibit B. cinereal mycelial growth. In contrast, when naked-dsRNAs targeting DCL1 and DCL2 were used, no significant mycelial growth inhibition was observed (Data not shown).

In this example, inventors showed that a single ME-dsRNA (500 ng/mL) or a combinational/dual ME-dsRNAs (1000 ng/mL) applications were significantly effective compared to empty-minicells for as long as 72 hpt (FIGS. 3E and 3F). This remarkable efficacy of ME-dsRNA could be explained, at least partially, by the ability of minicells to protect dsRNAs from enzymatic degradation as demonstrated above (FIG. 2B).

The robust efficacy of ME-DCL1 and ME-DCL2 was also confirmed at the molecular level using qRT-PCR. (FIG. 3F). This data not only showed a significant reduction in DCL1 and DCL2 transcript levels when the respective minicells were used, but inventors also observed a potential compensatory relationship between DCL1 and DCL2 expression. The downregulation of DCL1 transcripts, via ME-DCL1 treatment, was paralleled with an upregulation of DCL2 transcripts, and vice versa. The combination of ME-DCL1+2 treatment, resulted in a spike in expression of both genes at the 24 hpt, followed by a gradual, yet significant, decline in transcript levels of both genes at 48 and 72 hpt (FIG. 3F). This finding is consistent with the results in FIGS. 3C and 3D where the combined, but not the single application of ME-DCL 1 and 2 led to a complete inhibition of the mycelial growth of B. cinerea.

### Example 3. Effects of the Minicells Encapsulated dsRNAs (ME-dsRNAs) as a Biofungicide On Other Fruit-Rot Fungal Pathogens

To test the target specificity of the ME-dsRNAs as a biofungicide in other fungal pathogens, the same experiments described above in Example 2 were performed on Alternaria alternata and Penicillium expansum. The effects of the B. cinerea specific-ME-dsRNAs on the growth of two fruit-rot fungi, Alternaria alternata and Penicillium expansum were evaluated following the same methods and doses described above. Mycelial growth-inhibitory rates (IR, %), were calculated following the same equation of IR indicated above. Three biological replicates were used for each assay.

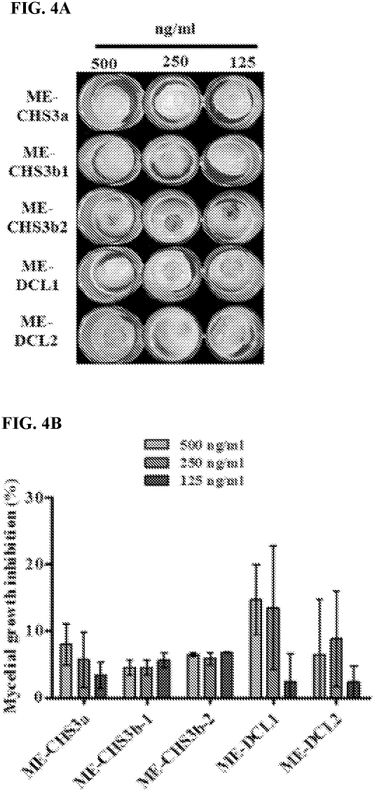

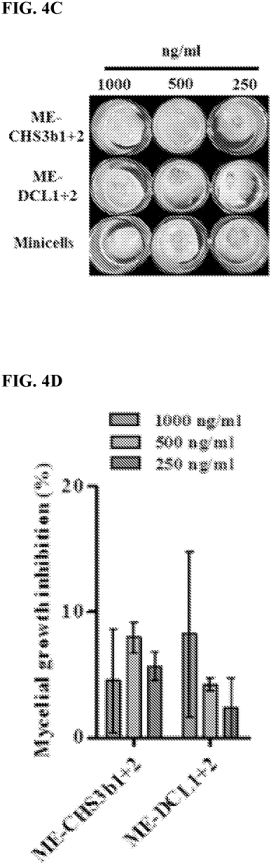

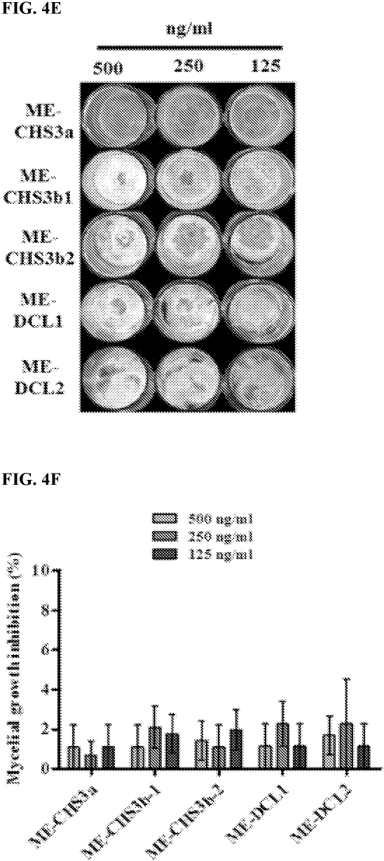

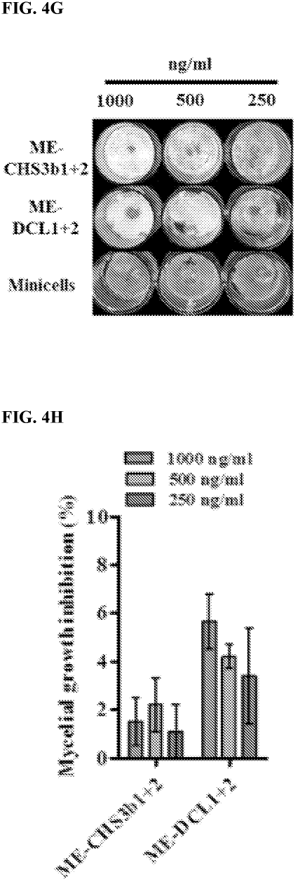

As shown in FIGS. 4A-4H, the target-specific inhibition by ME-dsRNAs was also confirmed in vitro by experimental results showing that ME-dsRNAs for B. cinerea failed to inhibit the mycelial growth of two other fruit-rot fungal pathogens, Alternaria alternata (FIGS. 4A-4D), and Penicillium expansum (FIGS. 4E-4H), which represents a major advantage of the ME-dsRNA as a selective class of biofungicides.

### Example 4. Topical Application of Minicells-Encapsulated dsRNAs (ME-dsRNAs) to Fruit-Bearing Plants under greenhouse Conditions

Exogenous application of ME-dsRNA was tested for crop and/or fruit protection against gray mold. Thus, ME-dsRNAs, targeting different B. cinerea genes, were tested for their efficacy as a topical spray application on fruit-bearing strawberries under greenhouse conditions.

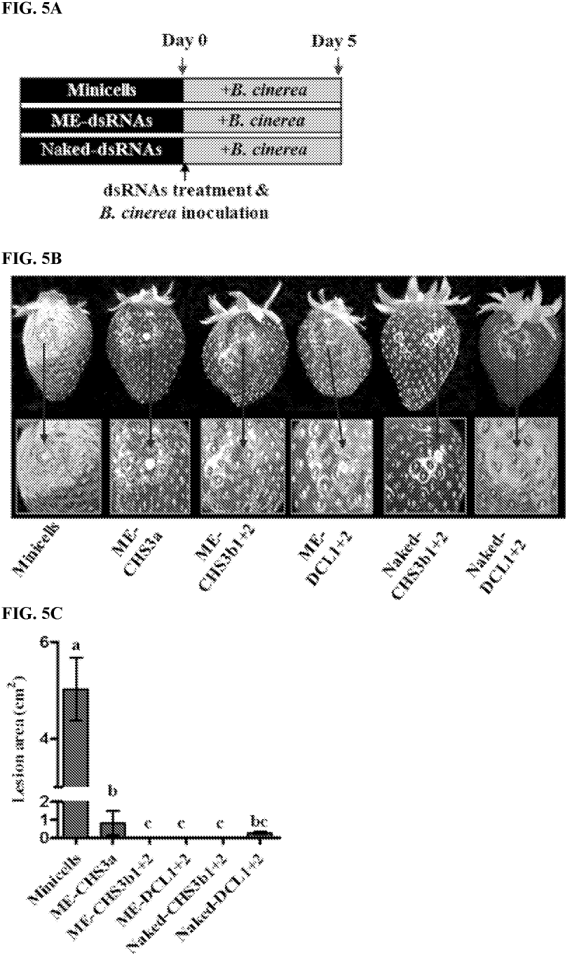

Strawberry (F. × ananassa cv. Earliglow) plant plugs were grown in a greenhouse at 24° C. in 7.5 L pots with natural light. The Botrytis cinerea was cultured in the PDA and mycelial plug (4 mm) from the leading edge of 5-d-old PDA plate was cut using a cork borer and used for the inoculation on strawberry fruits. The experiment was conducted with a completely randomized design with at least three biological replications. Three different pots, with a single plant per pot, were allocated for each treatment and at least one mature fruit/plant/pot was considered as a biological replication. The fruits were sprayed with a suspension containing ME-dsRNA, naked-dsRNA or the empty-minicells (control). The B. cinerea mycelium plug was inoculated 1 hour after the ME-dsRNA treatment in each treatment and at the fifth day post inoculation (FIG. 5A) , the samples were harvested, and the lesion diameter was measured in each fruit using the ImageJ software.

The bio-fungicidal capacity of naked or ME-dsRNA was observed when applied 1h before B. cinerea inoculation. ME-CHS3b1+2 and ME-DCL1+2 completely inhibited disease progression, while ME-CHS3a and naked-DCL1+2 showed minimal fungal growth (FIGS. 5B and 5C).

To assess the long-lasting effects of ME-dsRNA on disease development, the strawberry plants and the fungal mycelial disc were used as described above. However, in this experiment, the strawberries were sprayed first with ME-dsRNAs, the naked in-vitro synthesized dsRNAs or empty minicells 7 days prior to the B. cinerea inoculation. 5 days after the B. cinerea mycelial plugs was inoculated, the relationship of ME-dsRNAs and naked-dsRNAs treatment and the diseases severity was assessed as describe previously for the short-term at the fifth day post inoculation of B. cinerea.

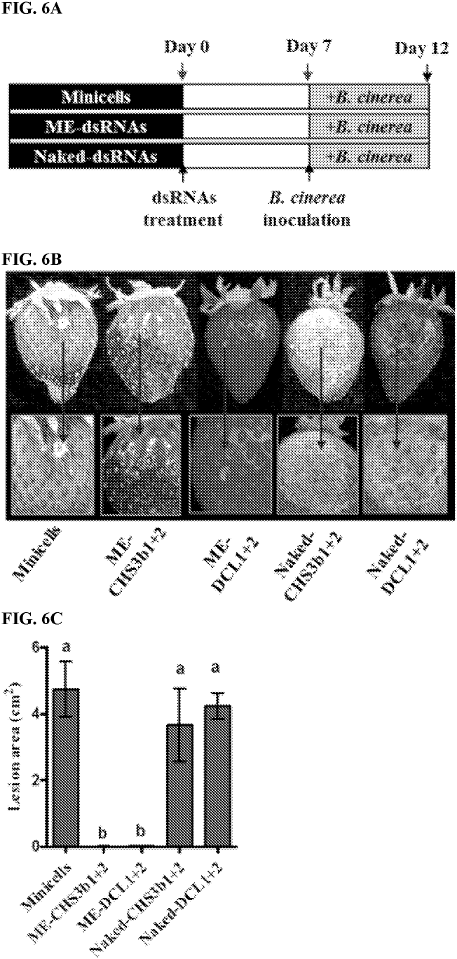

This experiment aimed to investigate the long-term efficacy (e.g. 3-5 days post treatment) of exogenous dsRNAs, as field stability was reported as a major limitation for RNAi-based fungicides (Wang et al 2016; Mitter et al 2017; Gan et al 2010). For this experiment, ME-, naked-dsRNAs or empty-minicells were applied 7 days prior to inoculation of B cinerea, and disease progression was analyzed at 5 days post inoculation (DPI) (FIG. 6A). While naked-dsRNAs failed to provide disease control and protection, showing a similar level of disease progression as the negative control (i.e. empty minicells), ME-dsRNAs provided complete protection against disease outbreaks, with no visible signs of gray mold (FIGS. 6B and 6C). These results demonstrate that ME-dsRNAs can extend the protection window against gray mold diseases by 5 to 12 days compared to naked-dsRNAs.

### SEQUENCES

The sequences described in this application are summarized in the following table.

| SEQ ID NO: | Sequence Type (size) | Description |
|---|---|---|
| 1 | Nucleic Acid (2737 bp) | cDNA of transcript transcribed from B. cinerea Chs3a gene |
| 2 | Nucleic Acid (417 bp) | Start site of B. cinerea Chs3a cDNA |
| 3 | Nucleic Acid | N-terminal region and catalytic site of B. cinerea Chs3a cDNA |
| SEQ ID NO: | Sequence Type (size) | Description |
| | (1344 bp) | |
| 4 | Nucleic Acid (1078 bp) | DsRNA target sequence 1 of B. cinerea Chs3a transcript (BCC1) |
| 5 | Nucleic Acid (956 bp) | DsRNA target sequence 2 of B. cinerea Chs3a transcript (BCC2) |
| 6 | Nucleic Acid (2715 bp) | cDNA of transcript transcribed from B. cinerea Chs3b gene |
| 7 | Nucleic Acid (608 bp) | DsRNA target sequence 1 of B. cinerea Chs3b transcript (BCC3) |
| 8 | Nucleic Acid (664 bp) | DsRNA target sequence 2 of B. cinerea Chs3b transcript (BCC4) |
| 9 | Nucleic Acid (7128 bp) | cDNA of transcript transcribed from B. cinerea Dcl1 gene |
| 10 | Nucleic Acid (508 bp) | Start site of B. cinerea Dcl1 cDNA |
| 11 | Nucleic Acid (244 bp) | Dimerization domain and Ribonuclease III C terminal domain of B. cinerea Dcl1 cDNA |
| 12 | Nucleic Acid (752 bp) | DsRNA target sequence of B. cinerea Dcl1 transcript |
| 13 | Nucleic Acid (5375 bp) | cDNA of transcript transcribed from B. cinerea Dcl2 gene |
| 14 | Nucleic Acid (237 bp) | Start site of B. cinerea Dcl2 cDNA |
| 15 | Nucleic Acid (600 bp) | Dimerization domain and Ribonuclease III C terminal domain of B. cinerea Dcl2 cDNA |
| 16 | Nucleic Acid (837 bp) | DsRNA target sequence of B. cinerea Dcl2 transcript |
| 17 | Nucleic Acid (4020 bp) | Botrytis cinerea B05.10 BcCHS3a (BcCHS3a), mRNA (XM 001557141) |
| 18 | Amino Acid (911 aa) | BcCHS3a Protein (XP_001557191) |
| 19 | Nucleic Acid (3075 bp) | Botrytis cinerea B05.10 BcCHS3b (BcCHS3b), mRNA (XM 001548495) |
| 20 | Amino Acid (905 aa) | BcCHS3b Protein (XP_001548545) |
| 21 | Nucleic Acid (6928 bp) | Botrytis cinerea B05.10 BcdcL1 (BcdcL1), mRNA (XM 024696562) |
| 22 | Amino Acid (1968 aa) | BcdcL1 [Botrytis cinerea B05.10] (XP_024552373) |
| 23 | Nucleic Acid (4872 bp) | Botrytis cinerea B05.10 BcdcL2 (BcdcL2), mRNA (XM 024697207) |
| 24 | Amino Acid (1523 aa) | BcdcL2 [Botrytis cinerea B05.10] (XP_024553022) |

### NUMBERED EMBODIMENTS OF THE DISCLOSURE

Notwithstanding the appended claims, the disclosure sets forth the following numbered embodiments:

1. A biocontrol composition comprising: a minicell comprising at least one polynucleotide encoding an inhibitory RNA molecule that are directed to at least one target sequence in a pathogen,
wherein the inhibitory RNA molecule causes downregulation of said at least one target sequence in the pathogen, and
wherein the pathogen or a disease caused by the pathogen is suppressed upon application of the composition compared to a control pathogen lacking the application of the composition.

2. The biocontrol composition according to embodiment 1, wherein said minicell is applied with at least one agriculturally suitable carrier.

3. The biocontrol composition according to embodiment 1, wherein the minicell is derived from a bacterial cell.

4. The biocontrol composition according to any one of embodiments 1-3, wherein the minicell is derived from a bacterial cell having a mislocalized cell division.

5. The biocontrol composition according to any one of embodiments 1-4, wherein the minicell is derived from a bacterial cell having a mislocalized cell division with deficient RNaseIII activity.

6. The biocontrol composition according to embodiment 1, wherein the inhibitory RNA molecule is an antisense RNA, a single-stranded RNA (ssRNA), a double-stranded RNA (dsRNA), a hairpin RNA (hpRNA), a short-hairpin RNA (shRNA), a small-interfering RNA (siRNA), a microRNA (miRNA), or combination thereof.

7. The biocontrol composition according to any one of embodiments 1-6, wherein the inhibitory RNA molecule is exogenously prepared and encapsulated by the minicell.

8. The biocontrol composition according to any one of embodiments 1-6, wherein the inhibitory RNA molecule is transcribed from at least one heterologous expression cassette comprising a promoter operably linked to the at least one polynucleotide within the minicell.

9. The biocontrol composition according to any one of embodiments 1-8, wherein the target sequence is selected from genes encoding Argonaute family proteins, chitin synthases, dicer-like proteins, proteins encoded by resistance genes, ABC family proteins, proteins encoded by meiotic silencing genes, sex-induced silencing proteins, proteins composed of the RISC Complex, and combination thereof.

10. The biocontrol composition according to embodiment 9, wherein the target sequence is a Chitin Synthase 3a gene, a Chitin Synthase 3b gene, a Dicer-like 1 gene, a Dicer-like 2 gene or combination thereof.

11. The biocontrol composition according to any one of embodiments 1-10, wherein the inhibitory RNA molecule targets a nucleic acid sequence encoding an amino acid sequence at least 90% identical to SEQ ID NO: 18 or 20.

12. The biocontrol composition according to any one of embodiments 1-10, wherein the inhibitory RNA molecule targets a nucleic acid sequence encoding an amino acid sequence at least 90% identical to SEQ ID NO:22 or 24.

13. The biocontrol composition according to embodiment 1, wherein the pathogen is selected from the group consisting of Albugo, Alternaria; Aphanomyces; Aspergillus; Ascochyta; Blumeria; Botrytis; Cercospora; Colletotrichum; Diplodia; Erysiphe; Fusarium; Gaeumannomyces; Helminthosporium; Leptosphaeria: Macrophomina; Magnaporthe; Melampsora; Mycospharella; Nectria; Penecillium; Peronospora; Phakopsora; Phialophora, Phoma; Phymatotrichum; Phytophthora; Plasmopara; Plasmodiophora; Podosphaera; Puccinia; Pythium; Pyrenophora; Pyricularia; Rhizoctonia; Scerotium; Sclerotinia; Septoria; Stemphylium; Thielaviopsis; Uncinula; Uromyces; Ustilago; Venturia; and Verticillium.

14. The biocontrol composition according to embodiment 13, wherein the pathogen is Alternaria alternata, Botrytis cinerea, Fusarium solani, Peronospora berteroae, Phakospora pachyrhizi, Penicillium expansum, Phialophora gregata, Phytophthora sojae, Sclerotinia sclerotiorum or Verticillium dahliae.

15. The biocontrol composition according to embodiment 14, wherein the pathogen is Botrytis cinerea.

16. The biocontrol composition according to embodiment 2, wherein the carrier is a solid, liquid, emulsion or powder form.

17. The biocontrol composition according to embodiment 16, wherein the carrier increases stability, wettability, or dispersability.

18. The biocontrol composition according to any one of embodiments 1-17, wherein the composition is applied to a subject by spraying, injecting, soaking, brushing, dressing, dripping, or coating in a solution.

19. The biocontrol composition according to embodiment 18, wherein the subject is a plant selected from the group consisting of: corn, wheat, rice, barley, oat, rye, sorghum, millet, sugar cane, strawberry, blueberry, raspberry, blackberry, apple, grape, pear, peach, melon, cucumber, pumpkin, squash, soybean, sugar beet, spinach, swiss chard, potato, eggplant, tomato, sunflower, safflower, gladiolus, cotton, canola, alfalfa, cannabis, Brassica, peanut, tobacco, banana, duckweed, pineapple, date, onion, cashew, pistachio, citrus, rose, almond, coffee, bean, legume, watermelon, squash, cabbage, turnip, mustard, cacti, pecan, flax, sweet potato, coconut, avocado, cantaloupe, vegetables, and herbs.

20. The biocontrol composition according to embodiment 19, wherein the subject is a strawberry plant.

21. A biocontrol composition comprising: a minicell comprising at least one polynucleotide encoding an inhibitory RNA molecule that targets a nucleic acid sequence encoding an amino acid sequence at least 90% identical to SEQ ID NO: 18, 20, 22 or 24,
wherein the sequence is present in a pathogen, and
wherein the pathogen or a disease caused by the pathogen is suppressed upon application of the composition compared to a control pathogen lacking the application of the composition.

22. The biocontrol composition according to embodiment 21, wherein said minicell is applied with at least one agriculturally suitable carrier.

23. The biocontrol composition according to embodiment 21, wherein the minicell is derived from a bacterial cell.

24. The biocontrol composition according to any one of embodiments 21-23, wherein the minicell is derived from a bacterial cell having a mislocalized cell division.

25. The biocontrol composition according to any one of embodiments 21-24, wherein the minicell is derived from a bacterial cell having a mislocalized cell division and having suppressed RNaseIII activity.

26. The biocontrol composition according to embodiment 21, wherein the inhibitory RNA molecule is an antisense RNA, a single-stranded RNA (ssRNA), a double-stranded RNA (dsRNA), a hairpin RNA (hpRNA), a short-hairpin RNA (shRNA), a small-interfering RNA (siRNA), a microRNA (miRNA), or combination thereof.

27. The biocontrol composition according to any one of embodiments 21-26, wherein the inhibitory RNA molecule is exogenously prepared and encapsulated by the minicell.

28. The biocontrol composition according to any one of embodiments 21-26, wherein the inhibitory RNA molecule is transcribed from at least one heterologous expression cassette comprising a promoter operably linked to the at least one polynucleotide within the minicell.

29. The biocontrol composition according to embodiment 21, wherein the pathogen is selected from the group consisting of Albugo, Alternaria; Aphanomyces; Aspergillus; Ascochyta; Blumeria; Botrytis; Cercospora; Colletotrichum; Diplodia; Erysiphe; Fusarium; Gaeumannomyces; Helminthosporium; Leptosphaeria: Macrophomina; Magnaporthe; Melampsora; Mycospharella; Nectria; Penecillium; Peronospora; Phakopsora; Phialophora, Phoma; Phymatotrichum; Phytophthora; Plasmopara; Plasmodiophora; Podosphaera; Puccinia; Pythium; Pyrenophora; Pyricularia; Rhizoctonia; Scerotium; Sclerotinia; Septoria; Stemphylium; Thielaviopsis; Uncinula; Uromyces; Ustilago; Venturia; and Verticillium.

30. The biocontrol composition according to embodiment 29, wherein the pathogen is Alternaria alternata, Botrytis cinerea, Fusarium solani, Peronospora berteroae, Phakospora pachyrhizi, Penicillium expansum, Phialophora gregata, Phytophthora sojae, Sclerotinia sclerotiorum or Verticillium dahliae.

31. The biocontrol composition according to embodiment 30, wherein the pathogen is Botrytis cinerea.

32. The biocontrol composition according to embodiment 22, wherein the carrier is a solid, liquid, emulsion or powder form.

33. The biocontrol composition according to embodiment 32, wherein the carrier increases stability, wettability, or dispersability.

34. The biocontrol composition according to any one of embodiments 21-33, wherein the composition is applied to a subject by spraying, injecting, soaking, brushing, dressing, dripping, or coating in a solution.

35. The biocontrol composition according to embodiment 34, wherein the subject is a plant selected from the group consisting of: corn, wheat, rice, barley, oat, rye, sorghum, millet, sugar cane, strawberry, blueberry, raspberry, blackberry, apple, grape, pear, peach, melon, cucumber, pumpkin, squash, soybean, sugar beet, spinach, swiss chard, potato, eggplant, tomato, sunflower, safflower, gladiolus, cotton, canola, alfalfa, cannabis, Brassica, peanut, tobacco, banana, duckweed, pineapple, date, onion, cashew, pistachio, citrus, rose, almond, coffee, bean, legume, watermelon, squash, cabbage, turnip, mustard, cacti, pecan, flax, sweet potato, coconut, avocado, cantaloupe, vegetables, and herbs.

36. The biocontrol composition according to embodiment 35, wherein the subject is a strawberry plant.

37. A method for suppressing, inhibiting, limiting, or controlling growth of or killing a pathogen, the method comprising the steps of:
a. introducing into a minicell at least one polynucleotide encoding an inhibitory RNA molecule that are directed to at least one target sequence in a pathogen,
b. applying the minicell to a subject; wherein the inhibitory RNA molecule causes downregulation of said at least one target sequence in the pathogen, and
wherein the pathogen or a disease caused by the pathogen is suppressed upon application of the composition compared to a control pathogen lacking the application of the composition.

38. The method according to embodiment 37, wherein said minicell is applied with at least one agriculturally suitable carrier.

39. The method according to embodiment 37, wherein the minicell is derived from a bacterial cell.

40. The method according to any one of embodiments 37-39, wherein the minicell is derived from a bacterial cell having a mislocalized cell division.

41. The method according to any one of embodiments 37-40, wherein the minicell is derived from a bacterial cell having a mislocalized cell division with deficient RNaseIII activity.

42. The method according to embodiment 37, wherein the inhibitory RNA molecule is an antisense RNA, a single-stranded RNA (ssRNA), a double-stranded RNA (dsRNA), a hairpin RNA (hpRNA), a short-hairpin RNA (shRNA), a small-interfering RNA (siRNA), a microRNA (miRNA), or combination thereof.

43. The method according to any one of embodiments 37-42, wherein the inhibitory RNA molecule is exogenously prepared and encapsulated by the minicell.

44. The method according to any one of embodiments 37-42, wherein the inhibitory RNA molecule is transcribed from at least one heterologous expression cassette comprising a promoter operably linked to the at least one polynucleotide within the minicell.

45. The method according to embodiment 37, wherein the target sequence is selected from genes encoding Argonaute family proteins, chitin synthases, dicer-like proteins, proteins encoded by resistance genes, ABC family proteins, proteins encoded by meiotic silencing genes, sex-induced silencing proteins, proteins composed of the RISC Complex, and combination thereof.

46. The method according to embodiment 45, the target sequence is a Chitin Synthase 3a gene, a Chitin Synthase 3b gene, a Dicer-like 1 gene, a Dicer-like 2 gene or combination thereof.

47. The method according to any one of embodiments 37-46, wherein the inhibitory RNA molecule targets a nucleic acid sequence encoding an amino acid sequence at least 90% identical to SEQ ID NO: 18 or 20.

48. The method according to any one of embodiments 37-46, wherein the inhibitory RNA molecule targets a nucleic acid sequence encoding an amino acid sequence at least 90% identical to SEQ ID NO:22 or 24.

49. The method according to embodiment 37, wherein the pathogen is selected from the group consisting of Albugo, Alternaria; Aphanomyces; Aspergillus; Ascochyta; Blumeria; Botrytis; Cercospora; Colletotrichum; Diplodia; Erysiphe; Fusarium; Gaeumannomyces; Helminthosporium; Leptosphaeria: Macrophomina; Magnaporthe; Melampsora; Mycospharella; Nectria; Penecillium; Peronospora; Phakopsora; Phialophora, Phoma; Phymatotrichum; Phytophthora; Plasmopara; Plasmodiophora; Podosphaera; Puccinia; Pythium; Pyrenophora; Pyricularia; Rhizoctonia; Scerotium; Sclerotinia; Septoria; Stemphylium; Thielaviopsis; Uncinula; Uromyces; Ustilago; Venturia; and Verticillium.

50. The method according to embodiment 49, wherein the pathogen is Alternaria alternata, Botrytis cinerea, Fusarium solani, Peronospora berteroae, Phakospora pachyrhizi, Penicillium expansum, Phialophora gregata, Phytophthora sojae, Sclerotinia sclerotiorum or Verticillium dahliae.

51. The method according to embodiment 50, wherein the pathogen is Botrytis cinerea.

52. The method according to embodiment 38, wherein the carrier is a solid, liquid, emulsion or powder form.

53. The method according to embodiment 52, wherein t the carrier increases stability, wettability, or dispersability.

54. The method according to embodiment 37, wherein the subject is a plant selected from the group consisting of: corn, wheat, rice, barley, oat, rye, sorghum, millet, sugar cane, strawberry, blueberry, raspberry, blackberry, apple, grape, pear, peach, melon, cucumber, pumpkin, squash, soybean, sugar beet, spinach, swiss chard, potato, eggplant, tomato, sunflower, safflower, gladiolus, cotton, canola, alfalfa, cannabis, Brassica, peanut, tobacco, banana, duckweed, pineapple, date, onion, cashew, pistachio, citrus, rose, almond, coffee, bean, legume, watermelon, squash, cabbage, turnip, mustard, cacti, pecan, flax, sweet potato, coconut, avocado, cantaloupe, vegetables, and herbs.

55. The method according to embodiment 54, wherein the subject is a strawberry plant.

56. The method according to embodiment 37, wherein the minicell is applied to the subject by spraying, injecting, soaking, brushing, dressing, dripping, or coating in a solution.

57. The method according to embodiment 37, further comprising the step of: introducing at least one additional polynucleotide encoding an inhibitory RNA molecule that are directed to at least one different target sequence in a pathogen.

58. The method according to embodiment 37, wherein the suppression lasts at least 1 hour, at least 12 hours, at least 1 day, at least 2 days, at least 3 days, at least 4 days, at least 5 days, at least one week or at least two weeks.

59. A method for preventing, treating or curing a disease or condition in a subject suffering therefrom a pathogen, the method comprising the steps of:
a. introducing into a minicell at least one polynucleotide encoding an inhibitory RNA molecule that are directed to at least one target sequence in a pathogen,
b. applying the minicell to the subject;
wherein the inhibitory RNA molecule causes downregulation of said at least one target sequence in the pathogen, and
wherein the pathogen or a disease caused by the pathogen is suppressed upon application of the composition compared to a control pathogen lacking the application of the composition.

60. The method according to embodiment 59, wherein said minicell is applied with at least one agriculturally suitable carrier.

61. The method according to embodiment 59, wherein the minicell is derived from a bacterial cell.

62. The method according to any one of embodiments 59-61, wherein the minicell is derived from a bacterial cell having a mislocalized cell division.

63. The method according to any one of embodiments 59-62, wherein the minicell is derived from a bacterial cell having a mislocalized cell division with deficient RNaseIII activity.

64. The method according to embodiment 59, wherein the inhibitory RNA molecule is an antisense RNA, a single-stranded RNA (ssRNA), a double-stranded RNA (dsRNA), a hairpin RNA (hpRNA), a short-hairpin RNA (shRNA), a small-interfering RNA (siRNA), a microRNA (miRNA), or combination thereof.

65. The method according to any one of embodiments 59-64, wherein the inhibitory RNA molecule is exogenously prepared and encapsulated by the minicell.

66. The method according to any one of embodiments 59-64, wherein the inhibitory RNA molecule is transcribed from at least one heterologous expression cassette comprising a promoter operably linked to the at least one polynucleotide within the minicell.

67. The method according to embodiment 59, wherein the target sequence is selected from genes encoding Argonaute family proteins, chitin synthases, dicer-like proteins, proteins encoded by resistance genes, ABC family proteins, proteins encoded by meiotic silencing genes, sex-induced silencing proteins, proteins composed of the RISC Complex, and combination thereof.

68. The method according to embodiment 67, the target sequence is a Chitin Synthase 3a gene, a Chitin Synthase 3b gene, a Dicer-like 1 gene, a Dicer-like 2 gene or combination thereof.

69. The method according to any one of embodiments 59-68, wherein the inhibitory RNA molecule targets a nucleic acid sequence encoding an amino acid sequence at least 90% identical to SEQ ID NO: 18 or 20.

70. The method according to any one of embodiments 59-68, wherein the inhibitory RNA molecule targets a nucleic acid sequence encoding an amino acid sequence at least 90% identical to SEQ ID NO:22 or 24.

71. The method according to embodiment 59, wherein the pathogen is selected from the group consisting of Albugo, Alternaria; Aphanomyces; Aspergillus; Ascochyta; Blumeria; Botrytis; Cercospora; Colletotrichum; Diplodia; Erysiphe; Fusarium; Gaeumannomyces; Helminthosporium; Leptosphaeria: Macrophomina; Magnaporthe; Melampsora; Mycospharella; Nectria; Penecillium; Peronospora; Phakopsora; Phialophora, Phoma; Phymatotrichum; Phytophthora; Plasmopara; Plasmodiophora; Podosphaera; Puccinia; Pythium; Pyrenophora; Pyricularia; Rhizoctonia; Scerotium; Sclerotinia; Septoria; Stemphylium; Thielaviopsis; Uncinula; Uromyces; Ustilago; Venturia; and Verticillium.

72. The method according to embodiment 71, wherein the pathogen is Alternaria alternata, Botrytis cinerea, Fusarium solani, Peronospora berteroae, Phakospora pachyrhizi, Penicillium expansum, Phialophora gregata, Phytophthora sojae, Sclerotinia sclerotiorum or Verticillium dahliae.

73. The method according to embodiment 72, wherein the pathogen is Botrytis cinerea.

74. The method according to embodiment 60, wherein the carrier is a solid, liquid, emulsion or powder form.

75. The method according to embodiment 74, wherein t the carrier increases stability, wettability, or dispersability.

76. The method according to embodiment 59, wherein the subject is a plant selected from the group consisting of: corn, wheat, rice, barley, oat, rye, sorghum, millet, sugar cane, strawberry, blueberry, raspberry, blackberry, apple, grape, pear, peach, melon, cucumber, pumpkin, squash, soybean, sugar beet, spinach, swiss chard, potato, eggplant, tomato, sunflower, safflower, gladiolus, cotton, canola, alfalfa, cannabis, Brassica, peanut, tobacco, banana, duckweed, pineapple, date, onion, cashew, pistachio, citrus, rose, almond, coffee, bean, legume, watermelon, squash, cabbage, turnip, mustard, cacti, pecan, flax, sweet potato, coconut, avocado, cantaloupe, vegetables, and herbs.

77. The method according to embodiment 76, wherein the subject is a strawberry plant.

78. The method according to embodiment 59, wherein the minicell is applied to the subject by spraying, injecting, soaking, brushing, dressing, dripping, or coating in a solution.

79. The method according to embodiment 59, further comprising the step of: introducing at least one additional polynucleotide encoding an inhibitory RNA molecule that are directed to at least one different target sequence in a pathogen.

80. The method according to embodiment 59, wherein the suppression lasts at least 1 hour, at least 12 hours, at least 1 day, at least 2 days, at least 3 days, at least 4 days, at least 5 days, at least one week or at least two weeks.

### INCORPORATION BY REFERENCE

All references, articles, publications, patents, patent publications, and patent applications cited herein are incorporated by reference in their entireties for all purposes. However, mention of any reference, article, publication, patent, patent publication, and patent application cited herein is not, and should not, be taken as an acknowledgment or any form of suggestion that they constitute valid prior art or form part of the common general knowledge in any country in the world. Specifically, the following applications are incorporated by reference: (1) PCT/US2018/030328, (2) US16/606,595, (3) PCT/US2018/030329, (4) US16/606,601, (5) PCT/US2018/052690, (6) US16,649,857, and (7) PCT/US2020/066706.

### REFERENCES

https://bsppjournals.onlinelibrary.wiley.com/doi/full/10.1111/j.1364-3703.2011.00783.x https://www.ncbi.nlm.nih.gov/pmc/articles/PMC4096153/

Boddy, L. (2016). Pathogens of autotrophs. The fungi (3rd ed.). London: Academic Press, 245-292.

Farquhar, S., Goff, N. M., Shadbeh, N., Samples, J., Ventura, S., Sanchez, V., & Davis, S.

. Occupational health and safety status of indigenous and Latino farmworkers in Oregon. Journal of Agricultural Safety and Health, 15, 89-102.

Fan, F., Hamada, M. S., Li, N., Li, G. Q., & Luo, C. X. (2017). Multiple fungicide resistance in Botrytis cinerea from greenhouse strawberries in Hubei Province, China. Plant Disease, 101 (4), 601-606.

Joel A. Abbey, David Percival, Lord Abbey, Samuel K. Asiedu, Balakrishnan Prithiviraj & Annemiek Schilder (2018): Biofungicides as alternative to synthetic fungicide control of grey mould (Botrytis cinerea) - prospects and challenges, Biocontrol Science and Technology, DOI: 10.1080/09583157.2018.1548574

Petrasch, S., Knapp, S.J., van Kan, J.A.L. & Blanco-Ulate, B. Grey mould of strawberry, a devastating disease caused by the ubiquitous necrotrophic fungal pathogen Botrytis cinerea. Mol. Plant Pathol. 20, 877-892 (2019).

Rupp, S., Weber, R.W.S., Rieger, D., Detzel, P. & Hahn, M. Spread of Botrytis cinerea strains with multiple fungicide resistance in german horticulture. Front. Microbiol. 7, 2075 (2017).

Fernández-Ortuño, D., Grabke, A., Li, X. & Schnabel, G. Independent emergence of resistance to seven chemical classes of fungicides in Botrytis cinerea. Phytopathology. 105, 424-432 (2015). Amiri, A., Heath, S.M. & Peres, N.A. Phenotypic characterization of multifungicide resistance in Botrytis cinerea isolates from strawberry fields in Florida. Plant Dis. 97, 393-401 (2013). Weber, R.W.S. Resistance of Botrytis cinerea to multiple fungicides in Northern German small-fruit production. Plant Dis. 95, 1263-1269 (2011).

Panebianco, A. et al. Detection of Botrytis cinerea field isolates with multiple fungicide resistance from table grape in Sicily. Crop. Protect. 77, 65-73 (2015).

Konstantinou, S. et al. Population structure, fungicide resistance profile, and sdhB mutation frequency of Botrytis cinerea from strawberry and greenhouse-grown tomato in Greece. Plant Dis. 99, 240-248 (2015).

Fire, A. et al. Potent and specific genetic interference by double-stranded RNA in Caenorhabditis elegans. Nature. 391, 806-811 (1998).

Baulcombe, D.C. VIGS, HIGS and FIGS: small RNA silencing in the interactions of viruses or filamentous organisms with their plant hosts. Curr. Opin. Plant Biol. 26, 141-146 (2015). Wang, M. et al. Bidirectional cross-kingdom RNAi and fungal uptake of external RNAs confer plant protection. Nat. Plants 2, 16151 (2016).

Xiong, F. et al. Host-induced gene silencing of BcTOR in Botrytis cinerea enhances plant resistance to grey mould. Mol. Plant Pathol. 20, 1722-1739 (2019).

Islam, M.T. & Sherif, S.M. RNAi-Based Biofungicides as a promising next-generation strategy for controlling devastating gray mold diseases. Int. J. Mol. Sci. 21, 2072 (2020).

Wang, M., Thomas,N. & Jin, H. Cross-kingdom RNA trafficking and environmental RNAi for powerful innovative pre- and post-harvest plant protection. Curr. Opin. Plant Biol. 38, 133-141 (2017).

Mcloughlin, A.G. et al. Developing new RNA interference technologies to control fungal pathogens. Can. J. Plant Pathol. 40, 325-335 (2018).

Mcloughlin, A.G. et al. Identification and application of exogenous dsRNA confers plant protection against Sclerotinia sclerotiorum and Botrytis cinerea. Sci. Rep. 8, 7320 (2018). Mitter, N. et al. Clay nanosheets for topical delivery of RNAi for sustained protection against plant viruses. Nat. Plants 3, 16207 (2017).

Farley, M.M., Hu, B., Margolin, W. & Liu, J. Minicells, back in fashion. J. Bacteriol. 198, 1186-1195 (2016).

MacDiarmid J.A. et al. Bacterially derived 400 nm particles for encapsulation and cancer cell targeting of chemotherapeutics. Cancer Cell 11, 431-445 (2007).

Carleton, H.A, Lara-Tejero, M., Liu, X. & Galán, J.E. Engineering the type III secretion system in non-replicating bacterial minicells for antigen delivery. Nat. Commun. 4, 1590 (2013). MacDiarmid, J. A. et al. Sequential treatment of drug-resistant tumors with targeted minicells containing siRNA or a cytotoxic drug. Nature Biotech. 27, 643-651 (2009).

Hale T.L., Sansonetti, P.J., Schad, P.A., Austin, S. & Formal, S.B. Characterization of virulence plasmids and plasmid-associated outer membrane proteins in Shigella flexneri, Shigella sonnei, and Escherichia coli. Infect. Immun. 40, 340-350 (1983).

Takiff, H.E., Chen, S. & D. L. Court. Genetic analysis of the rnc operon of Escherichia coli. J. Bacteriol. 171, 2581-2590 (1989).

Soulie, M. C. et al. Botrytis cinerea virulence is drastically reduced after disruption of chitin synthase class III gene (Bcchs3a). Cell Microbiol 8, 1310-1321 (2006).

Choquer, M., Boccara, M., Gonc,alves, I. R., Soulie, M. & Vidal-Cros, A. Survey of the Botrytis cinerea chitin synthase multigenic family through the analysis of six euascomycetes genomes. Eur. J. Biochem. 271, 2153-2164 (2004).

Choquer, M., Boccara, M., & Vidal-Cros, A. A semi-quantitative RT-PCR method to readily compare expression levels into Botrytis cinerea multigenic families. Curr. Genet. 43, 303- 309 (2003).

Nerva, L., Sandrini, M., Gambino, G. & Chitarra, W. Double-stranded RNAs (dsRNAs) as a sustainable tool against gray mold (Botrytis cinerea) in Grapevine: Effectiveness of different application methods in an open-air environment. Biomolecules. 10, 200 (2020).

Czech B. et al. An endogenous small interfering RNA pathway in Drosophila. Nature. 453, 798-802 (2008).

Wang, Z. et al. DrosophilaDicer-2 has an RNA interference-independent function that modulates Toll immune signaling. Sci. Adv. 1, e1500228 (2015).

Catalanotto, C. et al. Redundancy of the two dicer genes in transgene-induced posttranscrip-tional gene silencing in Neurospora crassa. Mol. Cell Biol. 24, 2536-2545 (2004).

Gan, D. et al. Bacterially expressed dsRNA protects maize against SCMV infection. Plant Cell Rep. 29, 1261-1268 (2010).

Nwokeoji, A. O., Kung, A. W., Kilby, P. M., Portwood, D. E., & Dickman, M. J. Purification and characterisation of dsRNA using ion pair reverse phase chromatography and mass spectrometry. J Chromatogr A. 1484, 14-25 (2017).

Sherif, S. et al. Simultaneous induction of jasmonic acid and disease-responsive genes signifies tolerance of American elm to Dutch elm disease. Sci. Rep. 6, 21934 (2016).

Livak, K. & Schmittgen, T. D. Analysis of relative gene expression data using real-time quantitative PCR and the 2^{-ΔΔC}tmethod. Methods, 25, 402-408 (2001).

Datsenko KA, Wanner BL. One-step inactivation of chromosomal genes in Escherichia coli K-12 using PCR products. Proc Natl Acad Sci U S A.2000;97(12):6640-5

What is claimed is:

1. A biocontrol composition comprising: a minicell comprising at least one polynucleotide encoding an inhibitory RNA molecule that are directed to at least one target sequence in a pathogen,
wherein the inhibitory RNA molecule causes downregulation of said at least one target sequence in the pathogen, and
wherein the pathogen or a disease caused by the pathogen is suppressed upon application of the composition compared to a control pathogen lacking the application of the composition.

2. The biocontrol composition according to claim 1, wherein said minicell is applied with at least one agriculturally suitable carrier.

3. The biocontrol composition according to claim 1, wherein the minicell is derived from a bacterial cell.

4. The biocontrol composition according to any one of claims 1-3, wherein the minicell is derived from a bacterial cell having a mislocalized cell division.

5. The biocontrol composition according to any one of claims 1-4, wherein the minicell is derived from a bacterial cell having a mislocalized cell division with deficient RNaseIII activity.

6. The biocontrol composition according to claim 1, wherein the inhibitory RNA molecule is an antisense RNA, a single-stranded RNA (ssRNA), a double-stranded RNA (dsRNA), a hairpin RNA (hpRNA), a short-hairpin RNA (shRNA), a small-interfering RNA (siRNA), a microRNA (miRNA), or combination thereof.

7. The biocontrol composition according to any one of claims 1-6, wherein the inhibitory RNA molecule is exogenously prepared and encapsulated by the minicell.

8. The biocontrol composition according to any one of claims 1-6, wherein the inhibitory RNA molecule is transcribed from at least one heterologous expression contruct comprising a promoter operably linked to the at least one polynucleotide within the minicell.

9. The biocontrol composition according to any one of claims 1-8, wherein the target sequence is selected from genes encoding Argonaute family proteins, chitin synthases, dicer-like proteins, proteins encoded by resistance genes, ABC family proteins, proteins encoded by meiotic silencing genes, sex-induced silencing proteins, proteins composed of the RISC Complex, and combination thereof.

10. The biocontrol composition according to claim 9, wherein the target sequence is a Chitin Synthase 3a gene, a Chitin Synthase 3b gene, a Dicer-like 1 gene, a Dicer-like 2 gene or combination thereof.

11. The biocontrol composition according to any one of claims 1-10, wherein the inhibitory RNA molecule targets a nucleic acid sequence encoding an amino acid sequence at least 90% identical to SEQ ID NO:18 or 20.

12. The biocontrol composition according to any one of claims 1-10, wherein the inhibitory RNA molecule targets a nucleic acid sequence encoding an amino acid sequence at least 90% identical to SEQ ID NO:22 or 24.

13. The biocontrol composition according to claim 1, wherein the pathogen is selected from the group consisting of Albugo, Alternaria; Aphanomyces; Aspergillus; Ascochyta; Blumeria; Botrytis; Cercospora; Colletotrichum; Diplodia; Erysiphe; Fusarium; Gaeumannomyces; Helminthosporium; Leptosphaeria: Macrophomina; Magnaporthe; Melampsora; Mycospharella; Nectria; Penecillium; Peronospora; Phakopsora; Phialophora, Phoma; Phymatotrichum; Phytophthora; Plasmopara; Plasmodiophora; Podosphaera; Puccinia; Pythium; Pyrenophora; Pyricularia; Rhizoctonia; Scerotium; Sclerotinia; Septoria; Stemphylium; Thielaviopsis; Uncinula; Uromyces; Ustilago; Venturia; and Verticillium.

14. The biocontrol composition according to claim 13, wherein the pathogen is Alternaria alternata, Botrytis cinerea, Fusarium solani, Peronospora berteroae, Phakospora pachyrhizi, Penicillium expansum, Phialophora gregata, Phytophthora sojae, Sclerotinia sclerotiorum or Verticillium dahliae.

15. The biocontrol composition according to claim 14, wherein the pathogen is Botrytis cinerea.

16. The biocontrol composition according to claim 2, wherein the carrier is a solid, liquid, emulsion or powder form.

17. The biocontrol composition according to claim 16, wherein the carrier increases stability, wettability, or dispersability.

18. The biocontrol composition according to any one of claims 1-17, wherein the composition is applied to a subject by spraying, injecting, soaking, brushing, dressing, dripping, or coating in a solution.

19. The biocontrol composition according to claim 18, wherein the subject is a plant selected from the group consisting of: corn, wheat, rice, barley, oat, rye, sorghum, millet, sugar cane, strawberry, blueberry, raspberry, blackberry, apple, grape, pear, peach, melon, cucumber, pumpkin, squash, soybean, sugar beet, spinach, swiss chard, potato, eggplant, tomato, sunflower, safflower, gladiolus, cotton, canola, alfalfa, cannabis, Brassica, peanut, tobacco, banana, duckweed, pineapple, date, onion, cashew, pistachio, citrus, rose, almond, coffee, bean, legume, watermelon, squash, cabbage, turnip, mustard, cacti, pecan, flax, sweet potato, coconut, avocado, cantaloupe, vegetables, and herbs.

20. The biocontrol composition according to claim 19, wherein the subject is a strawberry plant.

21. A biocontrol composition comprising: a minicell comprising at least one polynucleotide encoding an inhibitory RNA molecule that targets a nucleic acid sequence encoding an amino acid sequence at least 90% identical to SEQ ID NO:18, 20, 22 or 24,
wherein the sequence is present in a pathogen, and
wherein the pathogen or a disease caused by the pathogen is suppressed upon application of
the composition compared to a control pathogen lacking the application of the composition.

22. The biocontrol composition according to claim 21, wherein said minicell is applied with at least one agriculturally suitable carrier.

23. The biocontrol composition according to claim 21, wherein the minicell is derived from a bacterial cell.

24. The biocontrol composition according to any one of claims 21-23, wherein the minicell is derived from a bacterial cell having a mislocalized cell division.

25. The biocontrol composition according to any one of claims 21-24, wherein the minicell is derived from a bacterial cell having a mislocalized cell division and having suppressed RNaseIII activity.

26. The biocontrol composition according to claim 21, wherein the inhibitory RNA molecule is an antisense RNA, a single-stranded RNA (ssRNA), a double-stranded RNA (dsRNA), a hairpin RNA (hpRNA), a short-hairpin RNA (shRNA), a small-interfering RNA (siRNA), a microRNA (miRNA), or combination thereof.

27. The biocontrol composition according to any one of claims 21-26, wherein the inhibitory RNA molecule is exogenously prepared and encapsulated by the minicell.

28. The biocontrol composition according to any one of claims 21-26, wherein the inhibitory RNA molecule is transcribed from at least one heterologous expression cassette comprising a promoter operably linked to the at least one polynucleotide within the minicell.

29. The biocontrol composition according to claim 21, wherein the pathogen is selected from the group consisting of Albugo, Alternaria; Aphanomyces; Aspergillus; Ascochyta; Blumeria; Botrytis; Cercospora; Colletotrichum; Diplodia; Erysiphe; Fusarium; Gaeumannomyces; Helminthosporium; Leptosphaeria: Macrophomina; Magnaporthe; Melampsora; Mycospharella; Nectria; Penecillium; Peronospora; Phakopsora; Phialophora, Phoma; Phymatotrichum; Phytophthora; Plasmopara; Plasmodiophora; Podosphaera; Puccinia; Pythium; Pyrenophora; Pyricularia; Rhizoctonia; Scerotium; Sclerotinia; Septoria; Stemphylium; Thielaviopsis; Uncinula; Uromyces; Ustilago; Venturia; and Verticillium.

30. The biocontrol composition according to claim 29, wherein the pathogen is Alternaria alternata, Botrytis cinerea, Fusarium solani, Peronospora berteroae, Phakospora pachyrhizi, Penicillium expansum, Phialophora gregata, Phytophthora sojae, Sclerotinia sclerotiorum or Verticillium dahliae.

31. The biocontrol composition according to claim 30, wherein the pathogen is Botrytis cinerea.

32. The biocontrol composition according to claim 22, wherein the carrier is a solid, liquid, emulsion or powder form.

33. The biocontrol composition according to claim 32, wherein the carrier increases stability, wettability, or dispersability.

34. The biocontrol composition according to any one of claims 21-33, wherein the composition is applied to a subject by spraying, injecting, soaking, brushing, dressing, dripping, or coating in a solution.

35. The biocontrol composition according to claim 34, wherein the subject is a plant selected from the group consisting of: corn, wheat, rice, barley, oat, rye, sorghum, millet, sugar cane, strawberry, blueberry, raspberry, blackberry, apple, grape, pear, peach, melon, cucumber, pumpkin, squash, soybean, sugar beet, spinach, swiss chard, potato, eggplant, tomato, sunflower, safflower, gladiolus, cotton, canola, alfalfa, cannabis, Brassica, peanut, tobacco, banana, duckweed, pineapple, date, onion, cashew, pistachio, citrus, rose, almond, coffee, bean, legume, watermelon, squash, cabbage, turnip, mustard, cacti, pecan, flax, sweet potato, coconut, avocado, cantaloupe, vegetables, and herbs.

36. The biocontrol composition according to claim 35, wherein the subject is a strawberry plant.

37. A method for controlling a pathogen, the method comprising the steps of:
a. introducing into a minicell at least one polynucleotide encoding an inhibitory RNA molecule that are directed to at least one target sequence in a pathogen,
b. applying the minicell to a subject;
wherein the inhibitory RNA molecule causes downregulation of said at least one target sequence in the pathogen, and
wherein the pathogen or a disease caused by the pathogen is suppressed upon application of the composition compared to a control pathogen lacking the application of the composition.

38. The method according to claim 37, wherein said minicell is applied with at least one agriculturally suitable carrier.

39. The method according to claim 37, wherein the minicell is derived from a bacterial cell.

40. The method according to any one of claims 37-39, wherein the minicell is derived from a bacterial cell having a mislocalized cell division.

41. The method according to any one of claims 37-40, wherein the minicell is derived from a bacterial cell having a mislocalized cell division with deficient RNaseIII activity.

42. The method according to claim 37, wherein the inhibitory RNA molecule is an antisense RNA, a single-stranded RNA (ssRNA), a double-stranded RNA (dsRNA), a hairpin RNA (hpRNA), a short-hairpin RNA (shRNA), a small-interfering RNA (siRNA), a microRNA (miRNA), or combination thereof.

43. The method according to any one of claims 37-42, wherein the inhibitory RNA molecule is exogenously prepared and encapsulated by the minicell.

44. The method according to any one of claims 37-42, wherein the inhibitory RNA molecule is transcribed from at least one heterologous expression cassette comprising a promoter operably linked to the at least one polynucleotide within the minicell.

45. The method according to claim 37, wherein the target sequence is selected from genes encoding Argonaute family proteins, chitin synthases, dicer-like proteins, proteins encoded by resistance genes, ABC family proteins, proteins encoded by meiotic silencing genes, sex-induced silencing proteins, proteins composed of the RISC Complex, and combination thereof.

46. The method according to claim 45, the target sequence is a Chitin Synthase 3a gene, a Chitin Synthase 3b gene, a Dicer-like 1 gene, a Dicer-like 2 gene or combination thereof.

47. The method according to any one of claims 37-46, wherein the inhibitory RNA molecule targets a nucleic acid sequence encoding an amino acid sequence at least 90% identical to SEQ ID NO:18 or 20.

48. The method according to any one of claims 37-46, wherein the inhibitory RNA molecule targets a nucleic acid sequence encoding an amino acid sequence at least 90% identical to SEQ ID NO:22 or 24.

49. The method according to claim 37, wherein the pathogen is selected from the group consisting of Albugo, Alternaria; Aphanomyces; Aspergillus; Ascochyta; Blumeria; Botrytis; Cercospora; Colletotrichum; Diplodia; Erysiphe; Fusarium; Gaeumannomyces; Helminthosporium; Leptosphaeria: Macrophomina; Magnaporthe; Melampsora; Mycospharella; Nectria; Penecillium; Peronospora; Phakopsora; Phialophora, Phoma; Phymatotrichum; Phytophthora; Plasmopara; Plasmodiophora; Podosphaera; Puccinia; Pythium; Pyrenophora; Pyricularia; Rhizoctonia; Scerotium; Sclerotinia; Septoria; Stemphylium; Thielaviopsis; Uncinula; Uromyces; Ustilago; Venturia; and Verticillium.

50. The method according to claim 49, wherein the pathogen is Alternaria alternata, Botrytis cinerea, Fusarium solani, Peronospora berteroae, Phakospora pachyrhizi, Penicillium expansum, Phialophora gregata, Phytophthora sojae, Sclerotinia sclerotiorum or Verticillium dahliae.

51. The method according to claim 50, wherein the pathogen is Botrytis cinerea.

52. The method according to claim 38, wherein the carrier is a solid, liquid, emulsion or powder form.

53. The method according to claim 52, wherein t the carrier increases stability, wettability, or dispersability.

54. The method according to claim 37, wherein the subject is a plant selected from the group consisting of: corn, wheat, rice, barley, oat, rye, sorghum, millet, sugar cane, strawberry, blueberry, raspberry, blackberry, apple, grape, pear, peach, melon, cucumber, pumpkin, squash, soybean, sugar beet, spinach, swiss chard, potato, eggplant, tomato, sunflower, safflower, gladiolus, cotton, canola, alfalfa, cannabis, Brassica, peanut, tobacco, banana, duckweed, pineapple, date, onion, cashew, pistachio, citrus, rose, almond, coffee, bean, legume, watermelon, squash, cabbage, turnip, mustard, cacti, pecan, flax, sweet potato, coconut, avocado, cantaloupe, vegetables, and herbs.

55. The method according to claim 54, wherein the subject is a strawberry plant.

56. The method according to claim 37, wherein the minicell is applied to the subject by spraying, injecting, soaking, brushing, dressing, dripping, or coating in a solution.

57. The method according to claim 37, further comprising the step of: introducing at least one additional polynucleotide encoding an inhibitory RNA molecule that are directed to at least one different target sequence in a pathogen.

58. The method according to claim 37, wherein the suppression lasts at least 1 hour, at least 12 hours, at least 1 day, at least 2 days, at least 3 days, at least 4 days, at least 5 days, at least one week or at least two weeks.

59. A method for preventing, treating or curing a disease or condition in a subject suffering therefrom a pathogen, the method comprising the steps of:
a. introducing into a minicell at least one polynucleotide encoding an inhibitory RNA molecule that targets a nucleic acid sequence encoding an amino acid sequence at least 90% identical to SEQ ID NO:18, 20, 22 or 24,
b. applying the minicell to the subject;
wherein the inhibitory RNA molecule causes downregulation of said at least one target sequence in the pathogen, and
wherein the pathogen or a disease caused by the pathogen is suppressed upon application of the composition compared to a control pathogen lacking the application of the composition.


SEQUENCE LISTING (24 sequences)

SEQ ID NO: 1 - DNA, 2737 bp, Botrytis cinerea
atggcgtactctggaaacggaggctatgatgagcataagctccaagaccttccacctggagggggcaactaccatcaccctcaagatgaagaggaaggagctttacctttactcgattctggaggacatggcggcccattcaacagcccttacgactctcactcccaaggaggccttcgagcgaatactcctccagtcagacctgtttctgcctacagtcttacagaaacgtatgcgaacgatccacaaccatacagcagcgattacaactcatcccacacatacaatgagcagttagaagataacccatacccacaaaccgacactcctttgtcaagagccgggactacctccacagaggcatggagacaacgacaagcccctcaggctggaggtctcaaacgttatgctacaagaaaggttaagcttgtccagggaagcgtcttgagtgtcgatcatccagtacccagtgctatcaaaaatgcaattcagcaaaaatacagaaatgatttggagggtggcagcgaggaattcacacatatgcgttacaccgctgcaacttgcgatcccgatgaatttactctgaaaaatggttacaatctacgacctgccatgtacaatcgtcatactgagttgctcattgccgttacctattacaacgaagacaagcagttgactgcacgtactcttcacggtgtcatgcaaaacatccgtgatattgtcaacctcaagaaatctgatttctggaatgtcggcggaccagcatggcaaaaaattgttgtctgccttgtttttgatggtatcgatccttgcgacaaagacacactcgatgttcttgctacaattggtatctaccaagatggtgtcatgaagaaagatgttgatggaaaggagactgttgctcacgttttcgagtacacaacacaactttcggttactgccagtcaacaacttatccgcccaaccgagggtgatgccaactgtcttcctccggtgcaaatgatgttttgcttgaaggcaaagaacacaaagaagattaattcccatagatggctgttcaatgcatttggtcgtctccttaaccccgaagtttgcatccttttggatgccggtacgaagccaggacctaaatcgttgttgtctttgtgggaaggtttctacaacgacaaagatctaggaggtgcttgtggagagattcacgctatgttaggaaagggaggcaaaaagcttctgaacccgcttgttgcaggtcaaaacttcgagtataagatcagtaacattcttgacaagcctctagagagttctttcggatatgtcagtgtgttgccgggagctttctctgcataccgattccgagctattatgggacggcctcttgaacaatacttccacggagatcacactttgtccaagattcttggaaagaagggtatcgagggaatgaacattttcaagaagaacatgttcttggccgaggacagaattctctgtttcgagcttgtagccaaggctggtagcaagtggcatttgacttatatcaaggcagctaaaggagaaactgatgtacctgaaggagctgctgaattcattggtcaacgtagaagatggcttaacggttcctttgcagcaggaatgtattccatcatgcatttcggcagaatgtacaagagtggtcacaatattgtccgcatgttcttcttgcacattcaattcttatacaacgtcttctccaactttctcagttggttcatgttatcttctttctggctcactactactgttattatggatcttgtcggtacccctactgctgcttcggacactactgcccttcaccatggctggcctttcggtgatactgctacccccatcatcaacacgattctgaaatatctctacttgggattcctactcgttcaattcattctggctcttggtaatcggcctaagggatccaaggctacttatattgtctcctttgtcgtttttggtgttatcaatctctacgtccttgttctatccatgtatctggtcgttcgtgctttctcgtcaggagatagtctcgatactagtagtgcgaaagatttctttgagagtttcttcggagatagttcgagttccggagctggtatcattattcttgctttagctgctacatttggattgtacttcgttgcatccttcatgtatatggatccatggcacatgttccattctttcggaccgtacttgttgctcatgtcatcttacatcaacattctcatggtgtacgctttcagtaattggcacgatgtttcttggggtaccaagggatctgataaagcagaagctcttccgtcagctaaaactacgaaggttgatggtaaagcagcagtcattgaagaaatcgacagaccgcaagaggatattgatagtcagtttgaggcaactgtcaagcgtgctcttagtccatttgttcctgaagttgaagatgagagtaagaccttggaagattcttacaaatctttcagaaccaagcttttgatctcttgggtattctcaaacgcgttgttggctgttgctatcactagtgacagtgttgatggatttggtttcggaaatactgcatcaataagaactgctaagtttttcgaaattttgctttactcaacggctttcttggcacttattcgattcctcggatgttcttacttcttgttcaagtctggtatcatgtgctgcttcatgagacgttaaa

SEQ ID NO: 2 - DNA, 417 bp, Botrytis cinerea
atggcgtactctggaaacggaggctatgatgagcataagctccaagaccttccacctggagggggcaactaccatcaccctcaagatgaagaggaaggagctttacctttactcgattctggaggacatggcggcccattcaacagcccttacgactctcactcccaaggaggccttcgagcgaatactcctccagtcagacctgtttctgcctacagtcttacagaaacgtatgcgaacgatccacaaccatacagcagcgattacaactcatcccacacatacaatgagcagttagaagataacccatacccacaaaccgacactcctttgtcaagagccgggactacctccacagaggcatggagacaacgacaagcccctcaggctggaggtctcaaacgttatgctacaaga

SEQ ID NO: 3 - DNA, 1344 bp, Artificial Sequence
  Catalytic sites
aaggttaagcttgtccagggaagcgtcttgagtgtcgatcatccagtacccagtgctatcaaaaatgcaattcagcaaaaatacagaaatgatttggagggtggcagcgaggaattcacacatatgcgttacaccgctgcaacttgcgatcccgatgaatttactctgaaaaatggttacaatctacgacctgccatgtacaatcgtcatactgagttgctcattgccgttaccgccaactgtcttcctccggtgcaaatgatgttttgcttgaaggcaaagaacacaaagaagattaattcccatagatggctgttcaatgcatttggtcgtctccttaaccccgaagtttgcatccttttggatgccggtacgaagccaggacctaaatcgttgttgtctttgtgggaaggtttctacaacgacaaagatctaggaggtgcttgtggagagattcacgctatgttaggaaagggaggcaaaaagcttctgaacccgcttgttgcaggtcaaaacttcgagtataagatcagtaacattcttgacaagcctctagagagttctttcggatatgtcagtgtgttgccgggagctttctctgcataccgattccgagctattatgggacggcctcttgaacaatacttccacggagatcacactttgtccaagattcttggaaagaagggtatcgagggaatgaacattttcaagaagaacatgttcttggccgaggacagaattctctgtttcgagcttgtagccaaggctggtagcaagtggcatttgacttatatcaaggcagctaaaggagaaactgatgtacctgaaggagctgctgaattcattggtcaacgtagaagatggcttaacggttcctttgcagcaggaatgtattccatcatgcatttcggcagaatgtacaagagtggtcacaatattgtccgcatgttcttcttgcacattcaattcttatacaacgtcttctccaactttctcagttggttcatgttatcttctttctggctcactactactgttattatggatcttgtcggtacccctactgctgcttcggacactactgcccttcaccatggctggcctttcggtgatactgctacccccatcatcaacacgattctgaaatatctctacttgggattcctactcgttcaattcattctggctcttggtaatcggcctaagggatccaaggctacttatattgtctcctttgtcgtttttggtgttatcaatctctacgtccttgttctatccatgtatctggtcgttcgtgctttctcgtcaggagatagtctcgatactagtagtgcgaaagat

SEQ ID NO: 4 - DNA, 1078 bp, Artificial Sequence
  DsRNA Target Construct 1 (BCC1)
aaggttaagcttgtccagggaagcgtcttgagtgtcgatcatccagtacccagtgctatcaaaaatgcaattcagcaaaaatacagaaatgatttggagggtggcagcgaggaattcacacatatgcgttacaccgctgcaacttgcgatcccggccaactgtcttcctccggtgcaaatgatgttttgcttgaaggcaaagaacacaaagaagattaattcccatagatggctgttcaatgcatttggtcgtctccttaaccccgaagtttgcatccttttggatgccggtacgaagccaggacctaaatcgttgttgtctttgtgggaaggtttctacaacgacaaagatctaggaggtgcttgtggagagattcacgctatgttaggaaagggaggcaaaaagcttctgaacccgcttgttgcaggtcaaaacttcgagtataagatcagtaacattcttgacaagcctctagagagttctttcggatatgtcagtgtgttgccgggagctttctctgcataccgattccgagctattatgggacggcctcttgaacaatacttccacggagatcacactttgtccaagattcttggaaagaagggtatcgagggaatgaacattttcaagaagaacatgttcttggccgaggacagaattctctgtttcgagcttgtagccaaggctggtagcaagtggcatttgacttatatcaaggcagctaaaggagaaactgatgtacctgaaggagctgctgaattcattggtcaacgtagaagatggcttaacggttcctttgcagcaggaatgtattccatcatgcatttcggcagaatgtacaagagtggtcacaatattgtccgcatgttcttcttgcacattcaattcttatacaacgtcttctccaactttctcagttggttcatgttatcttctttctggctcactactactgttattatggatcttgtcggtacccctactgctgcttcggacactactgcccttcaccatggctggcctttcggtgatactgctacccccatcatcaacacgattctgaaatatctctac

SEQ ID NO: 5 - DNA, 956 bp, Artificial Sequence
  DsRNA Target Construct 2 (BCC2)
atggcgtactctggaaacggaggctatgatgagcataagctccaagaccttccacctggagggggcaactaccatcaccctcaagatgaagaggaaggagctttacctttactcgattctggaggacatggcggcccattcaacagcccttacgactctcactcccaaggaggccttcgagcgaatactcctccagtcagacctgtttctgcctacagtcttacagaaacgtatgcgaacgatccacaaccatacagcagcgattacaactcatcccacacatacaatgagcagttagaagataacccatacccacaaaccgacactcctttgtcaagagccgggactacctccacagaggcatggagacaacgacaagcccctcaggctggaggtctcaaacgttatgctacaagatgacaagcctctagagagttctttcggatatgtcagtgtgttgccgggagctttctctgcataccgattccgagctattatgggacggcctcttgaacaatacttccacggagatcacactttgtccaagattcttggaaagaagggtatcgagggaatgaacattttcaagaagaacatgttcttggccgaggacagaattctctgtttcgagcttgtagccaaggctggtagcaagtggcatttgacttatatcaaggcagctaaaggagaaactgatgtacctgaaggagctgctgaattcattggtcaacgtagaagatggcttaacggttcctttgcagcaggaatgtattccatcatgcatttcggcagaatgtacaagagtggtcacaatattgtccgcatgttcttcttgcacattcaattcttatacaacgtcttctccaactttctcagttggttcatgttatcttctttctggctcactactactgttattatggatcttgtcggtacccctactgctgcttcggaca

SEQ ID NO: 6 - DNA, 2715 bp, Artificial Sequence
  Bcchs3b gene Translated Exon DNA (cDNA)
atggaaacatcaaggagctggaatagttcggctgaaaactatcctatgaatgaacttattcaacattcaaacaattctgttactgatcctgctacacagttagataacactgatcatattagtgatgtacatcactcaaatgatgctcatgatgttggtgaaagtttgctatcagatactagtggctaccatccaacaacaccctatgtgactggtactgggcaggtacaacgtcaacgttcaccaaccagctcagtgaacggtgaggaatttttcggggatcaacaccatcttccagatatcgaccaggacttctacagaagccccgaagactcctttaatgcgcaagacatgccgtccgcgaacggtgctcgtggagctcttcggcgccgcgctactcgaaagattaaactgattcaaggcggtcagggatctgtcttgagcgcaaactaccctgtaccaagtgcaatcaagaatgcattacagccccagtacagagatatagagaatggtaccagcgagttctcagagatgcgatacaccgcggtaacttgcgacccgaatgattttaccttgatgaacggttatacactacgccagcacatatacaaccggcacaccgagcttttgattgcaatcacctactataatgaggacaaggtgctattcgctagaaccttgcatggtgtgatgaagaacattcgcgatattgtgaatctgaagaagtccaggttttggacgcagggttccgcagcatgggagaaaattgtcgtctgcctagtgttcgacggtttcgaaaaggcggatccaggtgtgctcgatctcctcacaaccgttggagtatatcaagacggcgtgcgaaaacaggatgtggatggaaaggagacgacagcccatgtcttcgagttcacgacacagttatccatcactcccgacctacagctttctcgtcccaattcggatgtggatgattccagtaacttgcctccagtacagctcctcttctgtctcaaacaaaagaataccaaaaaaatcaactcgcatcgttggttgttcaatgcctttggacggattctcaacccggaagtcaccattttgatcgacgcgggaacaaaacctggacctcgctctttattggccttatgggaggcattctataacgacaagaaccttggtggcgcatgtggtgagatccacgctatgcttgggagccggaactccaagttactaaatccgcttgtggcaatacagaatttcgaatacaagatttcgaatgttcttgacaaaccccttgagagctccttcggctacgtcactgttctccccggcgcattttcagcatatcgctttcgagcgatcatgggccgtcccttggagcaatattctcacggcgaccatacgttgttgtctggcaagaaaagtatcaacaacatgaatatttttaaggagaatatgttccttgcagaagatcgtatcctttgtttcgagctagtagtgaaagaaggacagaattggcacttgtcttatgtcaaagctgccaagggcgagacagacgtaccggaaggccctgctgagtttctcagccaacgccgccgttggctgaatggatcatttgcggcctcgctatactcgcttattcacttcggtcgcatgtacaaatccgggcacagtctgctccggatgataatgtttcatttccagctgctatacaacattgccaatgttatttttagttggttctctttatcatcctattggctcactaccactgtcatcatggatcttgtcggtacacctgtaactgcatcgaactatcacggctggccgtttggcgatactgcgtcccccatattcaaccacgttattgaatatatttaccttgcgtttgtgatcacacaattcattctggcccttggaaatcggccaaagggatcgcaagtgacctatctggtgttgttcgtggtgtttggatttattcagctctacatcatcattctgtcattctatctggtttaccgggcactacggactcccattggggaacagatcgacacgtcttcggccgcagcattctttgagagcatgtttggaggaacgggagtcgccggcgtgattttactagccttggtcaccatttacggcttgaactatgttgcctcgttcctctacttggatccttggcatatgtttcactccttcccgcagtatatcattctcgcttcgacatatattaacattttgatggtgtatgccttcaataactggcatgatgtttcttggggcacaaagggctcagatgaaagcgaaaagctcccttcggccaatgtgatcaaagactcgaaatccggtgtcgagatggtcgaagaagaggagatgcaacaaacagatattgaccagaagtttcaggagaccgtgctaagaactctggcccctgtcgaagtggaagttaccgtagaaaccaaggaggtcgatgacacgtataagtcgtttaggacccgcttggttgtctgctggatcctgtcgaatatgctgctggtcgggattgtcacctcggatgatttcgcatttcttggcgttggtcctctacgacccggaccgccatactacttcaaatttcttctatatgccactgctttcttgtcgattatcagattcttagggtttctttggtttctcggacgaacaaacattatgtactgctttgcgaagaggtaa

SEQ ID NO: 7 - DNA, 608 bp, Artificial Sequence
  DsRNA Target Construct 1
atggaaacatcaaggagctggaatagttcggctgaaaactatcctatgaatgaacttattcaacattcaaacaattctgttactgatcctgctacacagttagataacactgatcatattagtgatgtacatcactcaaatgatgctcatgatgttggtgaaagtttgctatcagatactagtggctaccatccaacaacaccctatgtgactggtactgggcaggtacaacgtcaacgttcaccaaccagctcagtgaacggtgaggaatttttcggggatcaacaccatcttccagatatcgaccaggacttctacagaagccccgaagactcctttaatgcgcaagacatgccgtccgcgaacggtgctcgtggagctcttcggcgccgcgctactcgaaagattaaactgattcaaggcggtcagggatctgtcttgagcgcaaactaccctgtaccaagtgcaatcaagaatgcattacagccccagtacagagatatagagaatggtaccagcgagttctcagagatgcgatacaccgcggtaacttgcgacccgaatgattttaccttgatgaacggttatacactacgccagcacatata

SEQ ID NO: 8 - DNA, 664 bp, Artificial Sequence
  DsRNA Target Construct 2
tcttctgtctcaaacaaaagaataccaaaaaaatcaactcgcatcgttggttgttcaatgcctttggacggattctcaacccggaagtcaccattttgatcgacgcgggaacaaaacctggacctcgctctttattggccttatgggaggcattctataacgacaagaaccttggtggcgcatgtggtgagatccacgctatgcttgggagccggaactccaagttactaaatccgcttgtggcaatacagaatttcgaatacaagatttcgaatgttcttgacaaaccccttgagagctccttcggctacgtcactgttctccccggcgcattttcagcatatcgctttcgagcgatcatgggccgtcccttggagcaatattctcacggcgaccatacgttgttgtctggcaagaaaagtatcaacaacatgaatatttttaaggagaatatgttccttgcagaagatcgtatcctttgtttcgagctagtagtgaaagaaggacagaattggcacttgtcttatgtcaaagctgccaagggcgagacagacgtaccggaaggccctgctgagtttctcagccaacgccgccgttggctgaatggatcatttgcggcctcgctatactcgcttattcacttcggtcgcatgtacaaatccgg

SEQ ID NO: 9 - DNA, 7128 bp, Botrytis cinerea
gagtttacatgttttatatatcttcaagctttctacattgggaaagatcttgggcatatttcaaccgcccgcaggagcacaagcgaatgacgtctcgcagatgacgagagacgcagcagcagcaaaaagtctctaccattggcgttgggtttctcctatcggatgaaacgctgggggtgaggggacgttcacttcttctgaataacacaaccagataacaataacaagaattcaatttctcccgcctctcacctcccaacacgctgaaaatatccattgcatcttggcaaacctctttctcaccctcttttcccttttcccgttcttatctctaccctttgacttctgcctgccagtgttgtgctgtagatctcatctcaccctgcagcatgcgctggaagaggtgcatgaatgcgacatcaaacaattctacactagtctaattgggtttgctcctggtattagcgatggaaactaatagaaaccaacaaacaggaagaaaaggcgtcactccttcagccgaagaggatcttctatcgtttgatgatattgttactgccgttccacctacaatcttgtcttcgtctgtcgctccatatacttctcgagataagataccttctgcatctggcaacggagatgctatagcagatgttagcagtggttacctcaaacaggctaccgtatcttctcattctgctcaagtccgatcatcttcaaacggcaatcaaggtgatgccaaaagttctccctctctttcacctgatagtaaactggaattcatctttgggcctcctttaagggagccagagaagccattctttaataaatcttcttattcgtttcgagattcgagtttgtttgctcaacaacccttcactaattttgggcaaggcctctcctcattgattgtcaacacggctaaaccattacttgaagcaagcttagtagatgagtctttaagatcttcaagtccagatcttattcaattcactcctccaaaacagaatttgcccaaggaggaaagagaaaaagctaaaaccctccaaattcacgcctcgaaatcgaattccagctcgttcgcgttcttatcattgaatcctttttcttcttcaagcttaccccgcacaacaggagggttgagcagaaatcgggcttcttcttctatggaaaattcgagaactctcgatccaaagatactcaaaccagttatcatcaataatcaccagggcgaatgcttccaagaggcttccagaacaggtatacctcaggctgatacttttgataaatcttcccttgctaagactgcggatatggatttgtcaccagtttctcaccatgcggatgtgcttgcgacgacggttagttctatatattcaggctctccgtgctggctaacaggtcctcaacaggtcactgcacagcattctgcaatagccgcccagaacgcagctcaaagctctaagatgccaggtcctgaagcttttttacttgccgaaaaggacgaggcaggttctcccgttgttatatcactgggttctgcaaaccaaattccttctggaaacatttctttgcagcttgattcaccatctctggaaaaccattctccaaatgtgaccccaatcaacaaagtccctacaccattcgcactttctacaaggacaaccgatgacgttttcgcagaacttaggcggcctttgcatccccaagctattcagagccagattgatatcaagacttcctcttgtgttgatagttataacacgaatgatgagattctagacaacaatcaaggttccaatcaaaaagatctgcatgttgttgaaaaggataaggaagaggaagaggaagaggatatgaaccaagccatacccgatatcaaacgtatctcagcacgaaaacaaaagaacgctgccatatttgacgtttttcttaaggaagctaccaaactaccaaagacagaaaagacttcacatgcgaatgatgaagcaattcagtctactaggtggttgattgaccaagcagaaaaacagcatattatagaaagtcccagggactatcaacttgaattgtttgagaaggcaaagaaacagaacattatagctgtacttgatacaggtaagcttttcttcgatcgatctctttttcccagtctgactaatatttttaggatctggcaagacattcattgcagttctcttacttcggtggatcatagaccaagagcttgaagatagagctattggcaagcctcatcgtgtttcattcttcctggtaagtcctacatactacatgacagatcaacactgacatgttagtaaggttgattctgtgaccctttgccatcaacaacactcagtactcagaaataatctcaatcagccaacagacatgatctgtgggcttatgggaactgacttatctgatcacataaagtggaagaaacgactggatacgaatatggtcattgtctgcactgcagaaattttgcgccaatgcctgcaccattcgtttgttacaatggctcaaataaatctgctaattttcgatgaagcccaccatgcaaagaaggatcatccttatgctaggattattaaagatttttatcgcaatgacacggaaaaggatatcgctctgcctaaaatatttgggatgacagcatcaccggtagatgctagagataatgtcaagaaagctgcggaagaacttgaaggtttgctacacagtcaaatatgtactgcagaagatcccagcttgctgcagtactcaatcaaaggtaaacctgagactcttgcctactatgatcccttgggcccgaaattcaatactcctctttatcttcaaatgctcccgcttctaaaagacaatcctatctttcggaagccatttgtatttgggacagaagccagtagaactctaggatcttggtgtgttgaccagatctggactttctgtcttcaagaagaagagtctaagaaactacaagcaaggacggagcaggcgcatcataagaagagagtcccggagccacttgaagtgctagagaaacgcaaggaacaacttgaacaagccaaatccattgtcgaaaatcacactttcgagccaccacactttgcatcaagattattggatgatttcacaacaaaagttcactattcgaataatttatctactaaagtcgttgctctcttgagtattctcaaagatcgtttccaacgacccaccaatgacaagtgtattgtatttgtcaaagaaagatacaccgcacgccttctagcctcacttctctccacacctgaagctgggacaccattcttgaaggctgcaccgctggtatgtaaccgattatatccactgaacacaactcctaacatttttacaggttggtactacgtctgcttcagccggggaaatgcatatcacatttagatcacaaactcttactatgcacaactttcgcaatggtaaaatcaactgccttatcgcaacatcagttgctgaagaaggtcttgacattcctgactgtaacctcgttgtcagattcgatttgtacaatacagtcattcagtacattcaatctagaggtcgtgctaggcatatcaattcaaggtactaccatatggtagagagccacaacgaggaacagattcgtacaatcaaagaggttttgaagcatgagaaaatgctaaagctttttgcttctgctcttccagaagatcgaaaattgaccggaaacaacttcaatatggattacttcctcagaaaagaacgaggccacagaatttaccctgtcccgaatagtgacgcaaaacttacttacagaatgagcttaacggtcctatctgccttcgttgactcacttcctcgagccccagagtcggttcttcgagtggattatgtcgtcacaactgtcgataagcagtttatctgtgaggccattttgccagaagaagcacccatacgcggagcaattggtcggccagcaacaactaaacaagtggccaaatgctcagcagcctttgaaacttgtgtgattctgcaccagaaaggatacatcaacgactacctactttctacatttaaaagatcagcacacatgatgagaaatgcacttttggctgtggatggaaagaagcaagaagcttatgatatgcagactaaaccaactttatggtcttcgaaagggaaacaaggcatattttatatgactgtcttgtctctcaaatctccagataatcttgacagagcatctcagccattgggcttactgacaagatcacccttgcctgatttgccagaatttgttcttcatttcggagcagggcgaaactctccaacctcgtgcgtacctctcgcttcctcaattacgctcgaaaaaaacaagcttgaccaagttaatatgttcaccctatgtttattccaagatgtgttcagtaaagcatacaaatcagatccggatagtatgccatactttctggttcctatcaactgcctgaatgctattgtcgactggaaatcacaaaacccaatgtcaataatcgattgggagacagttgaatatgtccaagacttcgagaataagcaagctgataagccatgggagcacaagccatggttaggaaagcctgacgattatttcaaagacaaattcataactgatccctttgacgggtctcgaaaattgtggtccgttggaatcacaaaagaatacagaccattggatccagtcccaccaaacacggcgcccaggaagggagctagaaagaacaatagtaatatcatggagtatagttgtagtctctgggcaaaggctagagcaaaacgaacttttgatgaagaacagcctgttattgaagcaacctacatttcacttcggagaaatttgcttgatgaatttgatggaggtgagctcgagacttcaaagaagtgttttattattttagaaccattgaaggtatcacctcttccaactaccgtggttgcaatggcctatcttttacctgcaattattcatcgagttgagtcatatctcattgctcttgaagcaacagacttgttacatcttgatatccgtcctgatcttgcgctagaggctgttaccaaggattccgacaattctggagagcatggtgaggaacagacaaactttcaacgtggaatgggcaataattatgaacgattggaatttcttggggactgcttcttgaagatgggaacgtcaatatctctatacggtctaaatcctgatagtgatgaattccgctaccatgttgatcgtatgtgtctgatttgcaacaaaaatctgttcaatacggctttgaaattagagctttacaaatacattcggtcggcagccttcaaccgacgagcttggtatcccgaaggccccgaattattaagaggaaagacagccacggcaccaaatacccacaagctcggcgataagtcagttgcagatgtttgtgaagcaatgattggagctgctttactaagccaccacgaaagcaagtccatggataatgcggttcgcgccgttactgaagttgtcaatagtgacaaccacaatgctgttgtatggtctgattattacaaattgtatgagaaaccaaaatggcaaactgctacagctacagctgcacaaatagatatggcaagacaagttgaaatgaaacatccatatcatttcaaacacccacgcctgttaagatcagctttcatccatccggcatacttgttcatctatgaacaaattccttgttatcaacgtctcgaatttttgggtgattcgctactcgatatggcatgtgtcaacttcctttttcacaaccacccaacaaaagatcctcagtggctcactgagcacaagatggctatagtatccaatcagtttcttggagctctttgtgtcaaattaggcttccacaaacatctactgacactcgattctcaagttcaaaaaatgattgcagattactcctcagatatcaatgaagctctcattcaagccaaaacggacgcaaagagagtcggcaaagtagaagatgattacgctcgtgattattggattgccgtccgtcaacctcctaaatgtcttcccgatattgtagaagcattcattggtgccatttttgtcgactctgagtatgactacggtgaagttgagaagttctttgaaatgcatatcagatggtactttgaggatatgggcatctacgatacctatgctaacaagcacccaaccactttccttactaatttcttgcaaaagaacatgggatgtgaggactgggcaccagttagtaaggaagtacctggagaggatggtagaaagaatgttgtagtttgcggggtcatcatacacaataaggtggtatcaactgccactgccgaaagtatgagatatgctagggtcggagcagcgaggaatgccttgagaaaattggagggaatgagtgtccgagaattcagggatgaatacgggtgctcatgtgaaggtgatgttgttgatgaagagggcaatattgaatttgttgaacgtgaagacgggatggaggggatcggtatgggatattgagaagaggttgttggcaatattttgaagaaagctgaggctgatttgaatggagattaaaaggggaatgaagctgcggggccaccgatagcacaaaaactactgaagatttgaagcacgttaaaattacactcaggaataaacggatggcaagcttttcgatcgcccaaacacggatctacgactacgagttacgcacgacatgatttagccttttgtgtgcaatgatgattagatagcattgcatttctcgaaattgacggcacgacttttacgggcagataatatcaaagattcctagtgagcaagcggtgatgatacgatgtcattccaaaagttttttcctcgcgaattttatttcatttcgaaggcatctttgcttagcagcatattcacctttgatgtcctctgtaggggatggagtctctaatctcgcggtcacaatgagacgtgatgcgctgcgaagtggtgacaatttccctttacttagaatagatcatgcacacatgcatgatgcatagctagctagttttttattcaatgatagtttaatgaca

SEQ ID NO: 10 - DNA, 508 bp, Botrytis cinerea
atggaaactaatagaaaccaacaaacaggaagaaaaggcgtcactccttcagccgaagaggatcttctatcgtttgatgatattgttactgccgttccacctacaatcttgtcttcgtctgtcgctccatatacttctcgagataagataccttctgcatctggcaacggagatgctatagcagatgttagcagtggttacctcaaacaggctaccgtatcttctcattctgctcaagtccgatcatcttcaaacggcaatcaaggtgatgccaaaagttctccctctctttcacctgatagtaaactggaattcatctttgggcctcctttaagggagccagagaagccattctttaataaatcttcttattcgtttcgagattcgagtttgtttgctcaacaacccttcactaattttgggcaaggcctctcctcattgattgtcaacacggctaaaccattacttgaagcaagcttagtagatgagtctttaagatcttcaagtc

SEQ ID NO: 11 - DNA, 244 bp, Artificial Sequence
  Catalytic Sites
ttcacgcctcgaaatcgaattccagctcgttcgcgttcttatcattgaatcctttttcttcttcaagcttaccccgcacaacaggagggttgatatcaagacttcctcttgtgttgatagttataacacgaatgatgagattctagacaacaatcaaggttccaatcaaaaagatctgcatgttgttgaaaaggataaggaagaggaagaggaagaggatatgaaccaagccatacccgatatc

SEQ ID NO: 12 - DNA, 752 bp, Artificial Sequence
  DCL1 Target construct
atggaaactaatagaaaccaacaaacaggaagaaaaggcgtcactccttcagccgaagaggatcttctatcgtttgatgatattgttactgccgttccacctacaatcttgtcttcgtctgtcgctccatatacttctcgagataagataccttctgcatctggcaacggagatgctatagcagatgttagcagtggttacctcaaacaggctaccgtatcttctcattctgctcaagtccgatcatcttcaaacggcaatcaaggtgatgccaaaagttctccctctctttcacctgatagtaaactggaattcatctttgggcctcctttaagggagccagagaagccattctttaataaatcttcttattcgtttcgagattcgagtttgtttgctcaacaacccttcactaattttgggcaaggcctctcctcattgattgtcaacacggctaaaccattacttgaagcaagcttagtagatgagtctttaagatcttcaagtcttcacgcctcgaaatcgaattccagctcgttcgcgttcttatcattgaatcctttttcttcttcaagcttaccccgcacaacaggagggttgatatcaagacttcctcttgtgttgatagttataacacgaatgatgagattctagacaacaatcaaggttccaatcaaaaagatctgcatgttgttgaaaaggataaggaagaggaagaggaagaggatatgaaccaagccatacccgatatc

SEQ ID NO: 13 - DNA, 5375 bp, Botrytis cinerea
gactgcctgtaccgacattctcagcatttcttatcgaaattgagaatgtcgtacaaaggtaaaaaaaaaaaaatgacaaatgaaagtccataaattcccaaaatggacaactattcgcggaaacaaaagaccccggtcgcaaaaacaaacgcctcatcttaaccacgactctctatctctcaaacccacatcgctcttccacaattcctcattcccagttagggtttttagtctaacgaatcattttcgctctccggcttctttcgaggacatggaatacacttcggaacctgacactgacccggatacacgcggtagccttatcgatggtcgagatgggattgaaggggatcttattgctttgacgtctggggaacgacttaatgagactgtagaggatttatgtagtgactcatcaggattgattgttgagaatgaagatgatgataacagcgcaggggagaagggagagattgtgatagtaacaccaagaacataccaactggaaatgttggaagagagtttgaaaaggaatgtcatcgttgcggtgggtgtatctcatacctgttggtggtacctgatgctaaaagttatattatagatggatacaggaagtggcaagacacatgtgtaagtcttctcctgccttggggtcaagattgggacaaatttggaagagatcgagtatactatagaccagatatttgaatatcgagtgctagtagagggcggcgttcacatcatgagtcgcagccatgtctttcttccgaaagcttgctacaatctgaccattcgatcatttagggccgttctccgaatactagcggaacttgagcggatgaagcctggcaaggtgggaagaaagttctaattcctcgttgttctcttcctgatcttgttgttgcagataatatggtttcttgcgcctaccgttgcgctctgtgctcagcatcacgaatatctccagctgaatattccctctgttttgatcaaaatgcttattggtgctgatggtgtggatcgatggacagagcagagacagtgggatacggtcttgaaggatgtcaaggtagtcgtatcttcctatcaagttcttctagatgcccttacacacggattcgtacgcatggggcgtctgtccttgatcatttttgatgaaggtgagtatcatgcacatctctacttgctgaacaagagattttttcagaccctgctaaacgtttatagcacataattgtgtaaataaagcgccaggggctaaaattatgaaatctttctatcatccgtataaatcgatattcccacttccccacattctgggcctctcggccagccctgtcatgagatccagtccacaatctttaagtgatatcgaggagactttggatgccatttgctgcacgccaaaaatacatcgagcagatcttcgccttcgagtaaagctaccacttctatctattatctactataccccagagtcaaatatcatcgtgacgaaaactgtggcgagcctgagaaagattgtgcaaagtctcaacattttcgaagacccctacgttttgacactaaaaaggagtgatagcgaaaaaagtcaacgtgagctggcgaaagtactcaagagttttaagacatatagtcaaacccaattaaagtcaatcgacaaaactagcaacgagattattcttgtagagctaggcccatgggctgcagattactatatctcaacagtggtgacgagatacttgaaggcaatgtcggcaaaggacactttcattgttgaagattcaccagctgccgagaagctatatattgccaaggctctcagacaagtcgaaatctctccttcaactctctcagatacaggcaaaatttctaacaaggttgaaaagctactggggataattgcgcaacagaagcctcccttttccgctattatatttgtccaagaaagagccacggtgtctgtgctagcccatctattatcgcatcatccattgacaaaggatcgttttaagattggaaccatggttggcacatccttaaatggcaagcgtacagaccaaataggagagcttgtcgatgttaatcaacaaaaagacactttgtcaagtttcaagcgtggaaaaattgatatccttatagctacaaatgtattggaagagggaattgatgttcctgcctgtaatctagtgatctgctttaggtaagtttaatgggatgtctaacattgcacccccagacgctatatggagacatttcatcattcacgttctcttcgtcccccctgtagctttagttccgcatatcacaccggaaacgtattcttataggcaatcactgacttaatgatgatatagtaaaccagcaaacctcaaatctttcgtacaaagacgagggcgagcaagacagcaagattctaagctgattcttcttgatgcttcaggtgataaagcgacaaattggcatgagcttgaaagaaaaatgcgagaggagtacggaaaggaaatgcgagaattgcaacacatctacgaaattgagacagctgatgaacagtcggaagatgatagggtcttgcgaatagaaagcactggggctcaattagaccttgacagtgctttaccacatctctatcatttctgttcagtcttaacaacaaaagattttgttgacctcaggccagacttcgtctactcctccgaactgggatcggaatatgttcgagcaaaggtcatcctgcctggatcggtttctaaacccctgcgagtccatgaaagccgcggatcgtggttgagcgagaggtcggctgcaaaagatgcagcgtttgaggcgtattccgcattatacagggggggcttagtgaatgataacctactgcccctgatggtgcacgacaaagtcatcgatgagttgacttcaaagcccgtggatactcgcgcgtctcttctggaggtgaaggaaagattaaatccatggattgacattgctagagcatggaaagaggcagaacaccatgctggaattgttcgcacatcggtaatgatcttcaatgggatgaagctggaactctgtcttccaattgatccaccggcaatacccccattaaagctttattgggatgctgacaccgagttctttgttgactttacaaacgatatcgagatcggcaccagcgagaatatgttggcacaggcgttgaacgataccaatctactattatcagatcgtggtcgtaaagttcacatccagtcacgtcgaacagttgtgcaatttatcttgcttcaagattcgggctcgctcagttcagattgttttccggttgaccccaacggtaatattaaaagtacaggttttatcagagaagtcggtaaactagaatcgccctacatctttgaaaaatggttgcccagtaagccaccgatcaatcaagtcaagaacccttatccagactttccagatgcaccagaagacgtcccatatctagctgtggttaaagtaagtcgccgtgcagactttttgcacaaggtacagaacgaaaaaccctcgtcattcactaaacaattctcgtctgttctacctgcctcgacatgtgtacaggatgtaatgcccgcacagttgtctcggttcggcatgatgattccttccatcacacaccacattgaggtgcaactcgttgtagaccgactatccaggaccatcctcaaggatctcgaaattagtgaccagagtcttattcagaccgccatcacacatgccagttattcgttagactcgaattatcagcgtctcgaatttctgggcgactcaattctcaaattgtgtacatcggtacaattggtggcagagcatctagattggcacgaaggatatttgtcggctatgaaggatcgtatcgtgtccaattcacggtcatcaagagcggcggctgaagtcggtttggatgagtatataatgaccaagaaattcacaggtgcaaaatggcgaccaatgtacgtggatgatctggtcgtcacagaacaaaaaacaagagaaatgtcctccaaaattctttccgacgttgtggaagcactcatcggcgcatgtatggtcgatggcggcattcctaaagcccttaaatctctacaactattcttcccagaactcaactggctccccttagaaactcgacaaatgacactctaccaacaagctgctaatgatctccatctacctatagctctccggcccgtcgagcaaatcctcgcatataccttcaccaaaaaatctctcctcgtcgaagccatgacgcacccctcttacaccagcggcacgcaatccctcgagcgactcgagttcctcggcgattccattctcgacaacatcatcgtcacagccatgtggtcgcactcgacgccgctctcccacttccacatgcatctcctgcgctctgcgctcgtcaacgccgatttcctcgcctttctctgcatggaaatgagcatcgaccaaaacgtcaccaatctgaccgaaggaaaaaaccatcgcatccacgaaacccactcgcgacgccgcgtttccctcgtcagttttctccgtcactcaagcgttcgtctctctatctatcaaaaagaagcgctttctcgccatgcagaattgcgcgatcagatcctcgaggcaatatacaccggtgatacattcccctgggctctattatcccgattggacgcgcggaaatttttctccgatatgattgagagtttgctgggcgcggtatggattgatagcggctcgatggaagtgtgcacgcagctgatcgaaagaatgggcgtcctgagatacatgcgacggattttgaaagatggcgtgcgcatcatgcatccgaaggaggaactgggcatcgtggccgattctgaaaacgtcaggtacgttttgcggcgggagaagatgggtggggatgctaccgaggtaaatgcggacgcggatgaagaggtacgcacggagtaccggtgcacagtatttgtgggcggggaggaaattgtagaggtgaggggtggagcgaggaaagaggagattcaggcaagggctgcggagcaggcggtgcggattttgaaggcgaggggtcatgagaagaggaatgggggtgcgggggaggggaaaaagagaaaatcgctggatgaatagagagcatttgtaggggaaggaggaaaaattgaggaggaggataagatgaattttgataaatttatttcctaacatcaggtcacaatctatgaattacatttgatagtattacgtatgccggtctgtacacaacacaaccatatagtaaggtatcaatcaaatgcgatggatagtcatttcaatttcttagtgaataattacaacgaaccagtaaaatagca

SEQ ID NO: 14 - DNA, 237 bp, Artificial Sequence
  Start Site
atgaaagtacacaagtttcccaagtggaccaccatccgcggtaacaaacggcctagatcccaaaagcaaactcctcatttaaaccacgatagtctttctcttaaacctacatctttatttcacaattctagctttcccgtaagagtgttttcgctgacgaaccattttaggagtccggcctcgtttgaggacatggagtacacaagcgaaccggacacggaccctgacactcgtggt

SEQ ID NO: 15 - DNA, 600 bp, Artificial Sequence
  Catalytic sites
tctctcatcgatggccgtgacggtatcgagggggacttaatagctttaacgtcaggagaacgacttaatgaaaccgttgaagacctctgttcagacagttctgggttaattgttgaaaacgaggacgatgataatagtgcaggtgaaaagggtgagattgtgatcgttactcctagaacatatcaactcgagatgttagaagaaagcctgaaacgcaacgttatcgttgccatggacaccggctcggggaaaacacacgtcgccgtgctccgaatactcgccgagctcgaacggatgaaaccgggcaagattatctggttcctggccccaacagtcgcactgtgtgcacaacatcatgaatacttgcaacttaacatcccgagtgttttaataaaaatgctgattggagccgatggtgtcgacaggtggaccgagcagcgtcaatgggaccgtttgctgcacgtcaaaaatacatcgaacagatcttcgcgtttgaatgatcctaccacttctatttgttgagtacaatcccccaaagtcaaataagttcatggcgtaagctttggagagcatgggaacgtctgtgtaaagtatcaacac

SEQ ID NO: 16 - DNA, 837 bp, Artificial Sequence
  DCL2 Target Construct
atgaaagtacacaagtttcccaagtggaccaccatccgcggtaacaaacggcctagatcccaaaagcaaactcctcatttaaaccacgatagtctttctcttaaacctacatctttatttcacaattctagctttcccgtaagagtgttttcgctgacgaaccattttaggagtccggcctcgtttgaggacatggagtacacaagcgaaccggacacggaccctgacactcgtggttctctcatcgatggccgtgacggtatcgagggggacttaatagctttaacgtcaggagaacgacttaatgaaaccgttgaagacctctgttcagacagttctgggttaattgttgaaaacgaggacgatgataatagtgcaggtgaaaagggtgagattgtgatcgttactcctagaacatatcaactcgagatgttagaagaaagcctgaaacgcaacgttatcgttgccatggacaccggctcggggaaaacacacgtcgccgtgctccgaatactcgccgagctcgaacggatgaaaccgggcaagattatctggttcctggccccaacagtcgcactgtgtgcacaacatcatgaatacttgcaacttaacatcccgagtgttttaataaaaatgctgattggagccgatggtgtcgacaggtggaccgagcagcgtcaatgggaccgtttgctgcacgtcaaaaatacatcgaacagatcttcgcgtttgaatgatcctaccacttctatttgttgagtacaatcccccaaagtcaaataagttcatggcgtaagctttggagagcatgggaacgtctgtgtaaagtatcaacac

SEQ ID NO: 17 - DNA, 4020 bp, Botrytis cinerea
gattcaccatcttcacacattcacattcacattcacaataataataataataagattctggcagtccaccgatgcaacacctttccatcttttcattggttcaaatctctttattccacctcatttcgccttgcttacgatcaaaaatcggtaaagaactctccagttgctttcagggttccgaataccaaaaaatcattgaagggatttgaaaatagcggagcccaagacaacaaactaaatcataaggaatccattccatccctcctatttttttattgacttcaagaatctgaaacagggttgcttttttattaccctttcggagcctttcttcatatcttcaccacttcaccacttcactttttgagaggagaagattcatccaccattcttttcttcttcgacttcttcttctcatcctctcttccacaccttggtaccatcgctggtgctttacattttgactgcgtgttctattcgtgaatcagaataaagaatttcgggttgaactacattcactcttttgcttcgcttgccaaggagagggtcgattcgatctatctgaccgtccgttcctgaagcccacggattgacaccgccatacgagaatatacctctgaatcttggacacaaccaacactcctcttttgttccatttctcatccatcaagtattgataaaatctcaaagattttgtcagtattgatactacacttaacatttgtataatctcgatcactcaagaaggggttcttgattattttctacaaccatggcgtactctggaaacggaggctatgatgagcataagctccaagaccttccacctggagggggcaactaccatcaccctcaagatgaagaggaaggagctttacctttactcgattctggaggacatggcggcccattcaacagcccttacgactctcactcccaaggaggccttcgagcgaatactcctccagtcagacctgtttctgcctacagtcttacagaaacgtatgcgaacgatccacaaccatacagcagcgattacaactcatcccacacatacaatgagcagttagaagataacccatacccacaaaccgacactcctttgtcaagagccgggactacctccacagaggcatggagacaacgacaagcccctcaggctggaggtctcaaacgttatgctacaagaaaggttaagcttgtccagggaagcgtcttgagtgtcgatcatccagtacccagtgctatcaaaaatgcaattcagcaaaaatacagaaatgatttggagggtggcagcgaggaattcacacatatgcgttacaccgctgcaacttgcgatcccgatgaatttactctgaaaaatggttacaatctacgacctgccatgtacaatcgtcatactgagttgctcattgccgttacctattacaacgaagacaagcagttgactgcacgtactcttcacggtgtcatgcaaaacatccgtgatattgtcaacctcaagaaatctgatttctggaatgtcggcggaccagcatggcaaaaaattgttgtctgccttgtttttgatggtatcgatccttgcgacaaagacacactcgatgttcttgctacaattggtatctaccaagatggtgtcatgaagaaagatgttgatggaaaggagactgttgctcacgttttcgagtacacaacacaactttcggttactgccagtcaacaacttatccgcccaaccgagggtgatgccaactgtcttcctccggtgcaaatgatgttttgcttgaaggcaaagaacacaaagaagattaattcccatagatggctgttcaatgcatttggtcgtctccttaaccccgaagtttgcatccttttggatgccggtacgaagccaggacctaaatcgttgttgtctttgtgggaaggtttctacaacgacaaagatctaggaggtgcttgtggagagattcacgctatgttaggaaagggaggcaaaaagcttctgaacccgcttgttgcaggtcaaaacttcgagtataagatcagtaacattcttgacaagcctctagagagttctttcggatatgtcagtgtgttgccgggagctttctctgcataccgattccgagctattatgggacggcctcttgaacaatacttccacggagatcacactttgtccaagattcttggaaagaagggtatcgagggaatgaacattttcaagaagaacatgttcttggccgaggacagaattctctgtttcgagcttgtagccaaggctggtagcaagtggcatttgacttatatcaaggcagctaaaggagaaactgatgtacctgaaggagctgctgaattcattggtcaacgtagaagatggcttaacggttcctttgcagcaggaatgtattccatcatgcatttcggcagaatgtacaagagtggtcacaatattgtccgcatgttcttcttgcacattcaattcttatacaacgtcttctccaactttctcagttggttcatgttatcttctttctggctcactactactgttattatggatcttgtcggtacccctactgctgcttcggacactactgcccttcaccatggctggcctttcggtgatactgctacccccatcatcaacacgattctgaaatatctctacttgggattcctactcgttcaattcattctggctcttggtaatcggcctaagggatccaaggctacttatattgtctcctttgtcgtttttggtgttatcaatctctacgtccttgttctatccatgtatctggtcgttcgtgctttctcgtcaggagatagtctcgatactagtagtgcgaaagatttctttgagagtttcttcggagatagttcgagttccggagctggtatcattattcttgctttagctgctacatttggattgtacttcgttgcatccttcatgtatatggatccatggcacatgttccattctttcggaccgtacttgttgctcatgtcatcttacatcaacattctcatggtgtacgctttcagtaattggcacgatgtttcttggggtaccaagggatctgataaagcagaagctcttccgtcagctaaaactacgaaggttgatggtaaagcagcagtcattgaagaaatcgacagaccgcaagaggatattgatagtcagtttgaggcaactgtcaagcgtgctcttagtccatttgttcctgaagttgaagatgagagtaagaccttggaagattcttacaaatctttcagaaccaagcttttgatctcttgggtattctcaaacgcgttgttggctgttgctatcactagtgacagtgttgatggatttggtttcggaaatactgcatcaataagaactgctaagtttttcgaaattttgctttactcaacggctttcttggcacttattcgattcctcggatgttcttacttcttgttcaagtctggtatcatgtgctgcttcatgagacgttaaacaagacttatatcatacatactctagaaggaaatatcggtactcgtaacattaaaaggttatgagtaatagatgtttctttttcacaaaattgggggggggggcggagaggaggatggaccctttatacggagcgtgagcacctatcattggaaaggaaacagcgcgtgaatctacatcaagcaaccatttttgcatttttgcttcacatatgggagttttcagattttttcttcttcctaggcggtctctctatattgcatgtttgagtaggcgtttccgtttaatactattgaatacccttatgaatgaataatcattcgtatctattgagctagatgacatctttcatgaagaatccaatgtgcactgcgtcaaaccatgagaaatttgcgtaaatgtttataatgatttgaatgtaccaactgaattgagaactttagatggtctgaatgcacagaatgatttgagtatattagatactttgaatacacaaaatgatttgacta

SEQ ID NO: 18 - protein, 911 aa, Botrytis cinerea
MAYSGNGGYDEHKLQDLPPGGGNYHHPQDEEEGALPLLDSGGHGGPFNSPYDSHSQGGLRANTPPVRPVSAYSLTETYANDPQPYSSDYNSSHTYNEQLEDNPYPQTDTPLSRAGTTSTEAWRQRQAPQAGGLKRYATRKVKLVQGSVLSVDHPVPSAIKNAIQQKYRNDLEGGSEEFTHMRYTAATCDPDEFTLKNGYNLRPAMYNRHTELLIAVTYYNEDKQLTARTLHGVMQNIRDIVNLKKSDFWNVGGPAWQKIVVCLVFDGIDPCDKDTLDVLATIGIYQDGVMKKDVDGKETVAHVFEYTTQLSVTASQQLIRPTEGDANCLPPVQMMFCLKAKNTKKINSHRWLFNAFGRLLNPEVCILLDAGTKPGPKSLLSLWEGFYNDKDLGGACGEIHAMLGKGGKKLLNPLVAGQNFEYKISNILDKPLESSFGYVSVLPGAFSAYRFRAIMGRPLEQYFHGDHTLSKILGKKGIEGMNIFKKNMFLAEDRILCFELVAKAGSKWHLTYIKAAKGETDVPEGAAEFIGQRRRWLNGSFAAGMYSIMHFGRMYKSGHNIVRMFFLHIQFLYNVFSNFLSWFMLSSFWLTTTVIMDLVGTPTAASDTTALHHGWPFGDTATPIINTILKYLYLGFLLVQFILALGNRPKGSKATYIVSFVVFGVINLYVLVLSMYLVVRAFSSGDSLDTSSAKDFFESFFGDSSSSGAGIIILALAATFGLYFVASFMYMDPWHMFHSFGPYLLLMSSYINILMVYAFSNWHDVSWGTKGSDKAEALPSAKTTKVDGKAAVIEEIDRPQEDIDSQFEATVKRALSPFVPEVEDESKTLEDSYKSFRTKLLISWVFSNALLAVAITSDSVDGFGFGNTASIRTAKFFEILLYSTAFLALIRFLGCSYFLFKSGIMCCFMRR

SEQ ID NO: 19 - DNA, 3075 bp, Botrytis cinerea
gaattgtcttgaacaaaaacgcatcttaaaggagtcggcttgactatttagctttcgagacttgcacagagtattcatgacggcgtctggctaagcatcctctgaaaacattcattcacatgccgatactatttttttttgcaatcagaagcagaaatttcttctcaaatatcccccgggtcatcttgactgctttttctccattctttccttttctatccttcattcttctcttctttaagagatcaaataaaaatggaaacatcaaggagctggaatagttcggctgaaaactatcctatgaatgaacttattcaacattcaaacaattctgttactgatcctgctacacagttagataacactgatcatattagtgatgtacatcactcaaatgatgctcatgatgttggtgaaagtttgctatcagatactagtggctaccatccaacaacaccctatgtgactggtactgggcaggtacaacgtcaacgttcaccaaccagctcagtgaacggtgaggaatttttcggggatcaacaccatcttccagatatcgaccaggacttctacagaagccccgaagactcctttaatgcgcaagacatgccgtccgcgaacggtgctcgtggagctcttcggcgccgcgctactcgaaagattaaactgattcaaggcggtcagggatctgtcttgagcgcaaactaccctgtaccaagtgcaatcaagaacgcattgcaaccccagtacagagatatagagaatggtaccagcgagttctcagagatgcgatacaccgcggtaacttgtgacccgaatgattttaccttgatgaacggttatacactacgccagcacatatacaaccggcacaccgagcttttgattgcaatcacctactataatgaggacaaggtgctattcgctagaaccttgcatggtgtgatgaagaacattcgcgatattgtgaatctgaagaagtccaggttttggacgcagggttccgcagcatgggagaaaattgtcgtctgcctagtgttcgacggtttcgaaaaggcggatccaggtgtgctcgatctcctcacaaccgttggagtatatcaagacggcgtgcgaaaacaggacgtggatggaaaggagacgacagcccatgtcttcgagttcacgacacagttatccatcactcccgacctacagctttctcgtcccaattcggatgtgcatgattccagtaacttgcctccagtacagctcctcttctgcctcaaacaaaagaatactaaaaagatcaactcgcatcgttggttattcaatgcctttggacggattctcaacccggaagtcaccattttgatcgacgcgggaacaaaacctggacctcgctctttattggccttatgggaggcattctataacgacaagaaccttggtggcgcatgtggtgagatccacgctatgcttgggagccggaactccaagttactaaatccgcttgtggcaatacagaatttcgaatacaagatttcgaatgttcttgacaaaccccttgagagctccttcggctacgtcactgttctccccggtgcattttcagcatatcgctttcgagcgatcatgggccgtcccttggagcaatattttcacggcgaccatacgttgttgtctggcaagaaaagtatcaacaacatgaatatttttaagaagaatatgttccttgcagaagatcgtatcctttgtttcgagctagtagtgaaagaaggacagaattggcacttgtcttatgtcaaagctgccaagggcgagacagacgtaccggaaggccctgctgagtttctcagccaacgccgccgttggctgaatggatcatttgctgcctcgctatactcgcttattcacttcggtcgcatgtacaaatccggacacagtctgctccggatgataatgtttcatttccagctgctatacaacattgccaatgttatttttagttggttctctttatcatcctattggctcaccaccactgtcatcatggatcttgtcggtacgcctgtagctgcatcgaactatcacggctggccgtttggcgatactgcgtcccccatattcaaccacgttattgaatatatttaccttgcgtttgtgatcacacaattcattctggcccttggaaatcggccaaagggatcgcaagtgacctatctggtgtcgtttgtggtgtttggatttattcagctctacatcatcattctgtcattttatctggtttaccgggcactacggactcccattggggaacagatcgacacgtcttcggccgcagcattctttgagagcatgtttggaggaacgggagtcgccggcgtgattttactagccttggtcaccatttacggcttgaactatgttgcctcgttcctctacttggatccttggcatatgtttcactccttcccgcagtatatcattctcgcttcgacatatattaacattttgatggtgtatgccttcaataactggcatgatgtttcttggggcacaaagggctcagatgaaagcgaaaagctcccttcggccaatgtgatcaaagactcgaaatccggtgtcgagatggtcgaagaagaggagatgcaacaaacagatattgaccagaagtttcaggagaccgtgctaagaactctggcccctgtcgaagtggaagttaccgtagaaaccaaggaggtcgatgacacgtataagtcgtttaggacccgcttggttgtctgctggatcctgtcgaatatgctgctggtcgggattgtcacctcgaatgatttcgcatttcttggcgttggtaaagcctctacgacccggacgccatactacttcaaatttcttctatatgccactgctttcttgtcgattatcagattcttagggtttctttggtttctcggacgaacaaacattatgcactgctttgcgaagaggtaatcgcatatacggcatggcatttgaagctggctgaagctagaaacatttcatttcttttaagttctaaagacaagatactcctaataataaacgaatgtagca

SEQ ID NO: 20 - protein, 905 aa, Botrytis cinerea
METSRSWNSSAENYPMNELIQHSNNSVTDPATQLDNTDHISDVHHSNDAHDVGESLLSDTSGYHPTTPYVTGTGQVQRQRSPTSSVNGEEFFGDQHHLPDIDQDFYRSPEDSFNAQDMPSANGARGALRRRATRKIKLIQGGQGSVLSANYPVPSAIKNALQPQYRDIENGTSEFSEMRYTAVTCDPNDFTLMNGYTLRQHIYNRHTELLIAITYYNEDKVLFARTLHGVMKNIRDIVNLKKSRFWTQGSAAWEKIVVCLVFDGFEKADPGVLDLLTTVGVYQDGVRKQDVDGKETTAHVFEFTTQLSITPDLQLSRPNSDVHDSSNLPPVQLLFCLKQKNTKKINSHRWLFNAFGRILNPEVTILIDAGTKPGPRSLLALWEAFYNDKNLGGACGEIHAMLGSRNSKLLNPLVAIQNFEYKISNVLDKPLESSFGYVTVLPGAFSAYRFRAIMGRPLEQYFHGDHTLLSGKKSINNMNIFKKNMFLAEDRILCFELVVKEGQNWHLSYVKAAKGETDVPEGPAEFLSQRRRWLNGSFAASLYSLIHFGRMYKSGHSLLRMIMFHFQLLYNIANVIFSWFSLSSYWLTTTVIMDLVGTPVAASNYHGWPFGDTASPIFNHVIEYIYLAFVITQFILALGNRPKGSQVTYLVSFVVFGFIQLYIIILSFYLVYRALRTPIGEQIDTSSAAAFFESMFGGTGVAGVILLALVTIYGLNYVASFLYLDPWHMFHSFPQYIILASTYINILMVYAFNNWHDVSWGTKGSDESEKLPSANVIKDSKSGVEMVEEEEMQQTDIDQKFQETVLRTLAPVEVEVTVETKEVDDTYKSFRTRLVVCWILSNMLLVGIVTSNDFAFLGVGKASTTRTPYYFKFLLYATAFLSIIRFLGFLWFLGRTNIMHCFAKR

SEQ ID NO: 21 - DNA, 6928 bp, Botrytis cinerea
gagtttacatgttttatatatcttcaagctttctacattgggaaagatcttgggcatatttcaaccgcccgcaggagcacaagcgaatgacgtctcgcagatgacgagagacgcagcagcagcaaaaagtctctaccattggcgttgggtttctcctatcggatgaaacgctgggggtgaggggacgttcacttcttctgaataacacaaccagataacaataacaagaattcaatttctcccgcctctcacctcccaacacgctgaaaatatccattgcatcttggcaaacctctttctcaccctcttttcccttttcccgttcttatctctaccctttgacttctgcctgccagtgttgtgctgtagatctcatctcaccctgcagcatgcgctggaagaggtgcatgaatgcgacatcaaacaattctacactagtctaattgggtttgctcctggtattagcgatggaaactaatagaaaccaacaaacaggaagaaaaggcgtcactccttcagccgaagaggatcttctatcgtttgatgatattgttactgccgttccacctacaatcttgtcttcgtctgtcgctccatatacttctcgagataagataccttctgcatctggcaacggagatgctatagcagatgttagcagtggttacctcaaacaggctaccgtatcttctcattctgctcaagtccgatcatcttcaaacggcaatcaaggtgatgccaaaagttctccctctctttcacctgatagtaaactggaattcatctttgggcctcctttaagggagccagagaagccattctttaataaatcttcttattcgtttcgagattcgagtttgtttgctcaacaacccttcactaattttgggcaaggcctctcctcattgattgtcaacacggctaaaccattacttgaagcaagcttagtagatgagtctttaagatcttcaagtccagatcttattcaattcactcctccaaaacagaatttgcccaaggaggaaagagaaaaagctaaaaccctccaaattcacgcctcgaaatcgaattccagctcgttcgcgttcttatcattgaatcctttttcttcttcaagcttaccccgcacaacaggagggttgagcagaaatcgggcttcttcttctatggaaaattcgagaactctcgatccaaagatactcaaaccagttatcatcaataatcaccagggcgaatgcttccaagaggcttccagaacaggtatacctcaggctgatacttttgataaatcttcccttgctaagactgcggatatggatttgtcaccagtttctcaccatgcggatgtgcttgcgacgacggtcactgcacagcattctgcaatagccgcccagaacgcagctcaaagctctaagatgccaggtcctgaagcttttttacttgccgaaaaggacgaggcaggttctcccgttgttatatcactgggttctgcaaaccaaattccttctggaaacatttctttgcagcttgattcaccatctctggaaaaccattctccaaatgtgaccccaatcaacaaagtccctacaccattcgcactttctacaaggacaaccgatgacgttttcgcagaacttaggcggcctttgcatccccaagctattcagagccagattgatatcaagacttcctcttgtgttgatagttataacacgaatgatgagattctagacaacaatcaaggttccaatcaaaaagatctgcatgttgttgaaaaggataaggaagaggaagaggaagaggatatgaaccaagccatacccgatatcaaacgtatctcagcacgaaaacaaaagaacgctgccatatttgacgtttttcttaaggaagctaccaaactaccaaagacagaaaagacttcacatgcgaatgatgaagcaattcagtctactaggtggttgattgaccaagcagaaaaacagcatattatagaaagtcccagggactatcaacttgaattgtttgagaaggcaaagaaacagaacattatagctgtacttgatacaggatctggcaagacattcattgcagttctcttacttcggtggatcatagaccaagagcttgaagatagagctattggcaagcctcatcgtgtttcattcttcctggttgattctgtgaccctttgccatcaacaacactcagtactcagaaataatctcaatcagccaacagacatgatctgtgggcttatgggaactgacttatctgatcacataaagtggaagaaacgactggatacgaatatggtcattgtctgcactgcagaaattttgcgccaatgcctgcaccattcgtttgttacaatggctcaaataaatctgctaattttcgatgaagcccaccatgcaaagaaggatcatccttatgctaggattattaaagatttttatcgcaatgacacggaaaaggatatcgctctgcctaaaatatttgggatgacagcatcaccggtagatgctagagataatgtcaagaaagctgcggaagaacttgaaggtttgctacacagtcaaatatgtactgcagaagatcccagcttgctgcagtactcaatcaaaggtaaacctgagactcttgcctactatgatcccttgggcccgaaattcaatactcctctttatcttcaaatgctcccgcttctaaaagacaatcctatctttcggaagccatttgtatttgggacagaagccagtagaactctaggatcttggtgtgttgaccagatctggactttctgtcttcaagaagaagagtctaagaaactacaagcaaggacggagcaggcgcatcataagaagagagtcccggagccacttgaagtgctagagaaacgcaaggaacaacttgaacaagccaaatccattgtcgaaaatcacactttcgagccaccacactttgcatcaagattattggatgatttcacaacaaaagttcactattcgaataatttatctactaaagtcgttgctctcttgagtattctcaaagatcgtttccaacgacccaccaatgacaagtgtattgtatttgtcaaagaaagatacaccgcacgccttctagcctcacttctctccacacctgaagctgggacaccattcttgaaggctgcaccgctggttggtactacgtctgcttcagccggggaaatgcatatcacatttagatcacaaactcttactatgcacaactttcgcaatggtaaaatcaactgccttatcgcaacatcagttgctgaagaaggtcttgacattcctgactgtaacctcgttgtcagattcgatttgtacaatacagtcattcagtacattcaatctagaggtcgtgctaggcatatcaattcaaggtactaccatatggtagagagccacaacgaggaacagattcgtacaatcaaagaggttttgaagcatgagaaaatgctaaagctttttgcttctgctcttccagaagatcgaaaattgaccggaaacaacttcaatatggattacttcctcagaaaagaacgaggccacagaatttaccctgtcccgaatagtgacgcaaaacttacttacagaatgagcttaacggtcctatctgccttcgttgactcacttcctcgagccccagagtcggttcttcgagtggattatgtcgtcacaactgtcgataagcagtttatctgtgaggccattttgccagaagaagcacccatacgcggagcaattggtcggccagcaacaactaaacaagtggccaaatgctcagcagcctttgaaacttgtgtgattctgcaccagaaaggatacatcaacgactacctactttctacatttaaaagatcagcacacatgatgagaaatgcacttttggctgtggatggaaagaagcaagaagcttatgatatgcagactaaaccaactttatggtcttcgaaagggaaacaaggcatattttatatgactgtcttgtctctcaaatctccagataatcttgacagagcatctcagccattgggcttactgacaagatcacccttgcctgatttgccagaatttgttcttcatttcggagcagggcgaaactctccaacctcgtgcgtacctctcgcttcctcaattacgctcgaaaaaaacaagcttgaccaagttaatatgttcaccctatgtttattccaagatgtgttcagtaaagcatacaaatcagatccggatagtatgccatactttctggttcctatcaactgcctgaatgctattgtcgactggaaatcacaaaacccaatgtcaataatcgattgggagacagttgaatatgtccaagacttcgagaataagcaagctgataagccatgggagcacaagccatggttaggaaagcctgacgattatttcaaagacaaattcataactgatccctttgacgggtctcgaaaattgtggtccgttggaatcacaaaagaatacagaccattggatccagtcccaccaaacacggcgcccaggaagggagctagaaagaacaatagtaatatcatggagtatagttgtagtctctgggcaaaggctagagcaaaacgaacttttgatgaagaacagcctgttattgaagcaacctacatttcacttcggagaaatttgcttgatgaatttgatggaggtgagctcgagacttcaaagaagtgttttattattttagaaccattgaaggtatcacctcttccaactaccgtggttgcaatggcctatcttttacctgcaattattcatcgagttgagtcatatctcattgctcttgaagcaacagacttgttacatcttgatatccgtcctgatcttgcgctagaggctgttaccaaggattccgacaattctggagagcatggtgaggaacagacaaactttcaacgtggaatgggcaataattatgaacgattggaatttcttggggactgcttcttgaagatgggaacgtcaatatctctatacggtctaaatcctgatagtgatgaattccgctaccatgttgatcgtatgtgtctgatttgcaacaaaaatctgttcaatacggctttgaaattagagctttacaaatacattcggtcggcagccttcaaccgacgagcttggtatcccgaaggccccgaattattaagaggaaagacagccacggcaccaaatacccacaagctcggcgataagtcagttgcagatgtttgtgaagcaatgattggagctgctttactaagccaccacgaaagcaagtccatggataatgcggttcgcgccgttactgaagttgtcaatagtgacaaccacaatgctgttgtatggtctgattattacaaattgtatgagaaaccaaaatggcaaactgctacagctacagctgcacaaatagatatggcaagacaagttgaaatgaaacatccatatcatttcaaacacccacgcctgttaagatcagctttcatccatccggcatacttgttcatctatgaacaaattccttgttatcaacgtctcgaatttttgggtgattcgctactcgatatggcatgtgtcaacttcctttttcacaaccacccaacaaaagatcctcagtggctcactgagcacaagatggctatagtatccaatcagtttcttggagctctttgtgtcaaattaggcttccacaaacatctactgacactcgattctcaagttcaaaaaatgattgcagattactcctcagatatcaatgaagctctcattcaagccaaaacggacgcaaagagagtcggcaaagtagaagatgattacgctcgtgattattggattgccgtccgtcaacctcctaaatgtcttcccgatattgtagaagcattcattggtgccatttttgtcgactctgagtatgactacggtgaagttgagaagttctttgaaatgcatatcagatggtactttgaggatatgggcatctacgatacctatgctaacaagcacccaaccactttccttactaatttcttgcaaaagaacatgggatgtgaggactgggcaccagttagtaaggaagtacctggagaggatggtagaaagaatgttgtagtttgcggggtcatcatacacaataaggtggtatcaactgccactgccgaaagtatgagatatgctagggtcggagcagcgaggaatgccttgagaaaattggagggaatgagtgtccgagaattcagggatgaatacgggtgctcatgtgaaggtgatgttgttgatgaagagggcaatattgaatttgttgaacgtgaagacgggatggaggggatcggtatgggatattgagaagaggttgttggcaatattttgaagaaagctgaggctgatttgaatggagattaaaaggggaatgaagctgcggggccaccgatagcacaaaaactactgaagatttgaagcacgttaaaattacactcaggaataaacggatggcaagcttttcgatcgcccaaacacggatctacgactacgagttacgcacgacatgatttagccttttgtgtgcaatgatgattagatagcattgcatttctcgaaattgacggcacgacttttacgggcagataatatcaaagattcctagtgagcaagcggtgatgatacgatgtcattccaaaagttttttcctcgcgaattttatttcatttcgaaggcatctttgcttagcagcatattcacctttgatgtcctctgtaggggatggagtctctaatctcgcggtcacaatgagacgtgatgcgctgcgaagtggtgacaatttccctttacttagaatagatcatgcacacatgcatgatgcatagctagctagttttttattcaatgatagtttaatgaca

SEQ ID NO: 22 - protein, 1968 aa, Botrytis cinerea
METNRNQQTGRKGVTPSAEEDLLSFDDIVTAVPPTILSSSVAPYTSRDKIPSASGNGDAIADVSSGYLKQATVSSHSAQVRSSSNGNQGDAKSSPSLSPDSKLEFIFGPPLREPEKPFFNKSSYSFRDSSLFAQQPFTNFGQGLSSLIVNTAKPLLEASLVDESLRSSSPDLIQFTPPKQNLPKEEREKAKTLQIHASKSNSSSFAFLSLNPFSSSSLPRTTGGLSRNRASSSMENSRTLDPKILKPVIINNHQGECFQEASRTGIPQADTFDKSSLAKTADMDLSPVSHHADVLATTVTAQHSAIAAQNAAQSSKMPGPEAFLLAEKDEAGSPVVISLGSANQIPSGNISLQLDSPSLENHSPNVTPINKVPTPFALSTRTTDDVFAELRRPLHPQAIQSQIDIKTSSCVDSYNTNDEILDNNQGSNQKDLHVVEKDKEEEEEEDMNQAIPDIKRISARKQKNAAIFDVFLKEATKLPKTEKTSHANDEAIQSTRWLIDQAEKQHIIESPRDYQLELFEKAKKQNIIAVLDTGSGKTFIAVLLLRWIIDQELEDRAIGKPHRVSFFLVDSVTLCHQQHSVLRNNLNQPTDMICGLMGTDLSDHIKWKKRLDTNMVIVCTAEILRQCLHHSFVTMAQINLLIFDEAHHAKKDHPYARIIKDFYRNDTEKDIALPKIFGMTASPVDARDNVKKAAEELEGLLHSQICTAEDPSLLQYSIKGKPETLAYYDPLGPKFNTPLYLQMLPLLKDNPIFRKPFVFGTEASRTLGSWCVDQIWTFCLQEEESKKLQARTEQAHHKKRVPEPLEVLEKRKEQLEQAKSIVENHTFEPPHFASRLLDDFTTKVHYSNNLSTKVVALLSILKDRFQRPTNDKCIVFVKERYTARLLASLLSTPEAGTPFLKAAPLVGTTSASAGEMHITFRSQTLTMHNFRNGKINCLIATSVAEEGLDIPDCNLVVRFDLYNTVIQYIQSRGRARHINSRYYHMVESHNEEQIRTIKEVLKHEKMLKLFASALPEDRKLTGNNFNMDYFLRKERGHRIYPVPNSDAKLTYRMSLTVLSAFVDSLPRAPESVLRVDYVVTTVDKQFICEAILPEEAPIRGAIGRPATTKQVAKCSAAFETCVILHQKGYINDYLLSTFKRSAHMMRNALLAVDGKKQEAYDMQTKPTLWSSKGKQGIFYMTVLSLKSPDNLDRASQPLGLLTRSPLPDLPEFVLHFGAGRNSPTSCVPLASSITLEKNKLDQVNMFTLCLFQDVFSKAYKSDPDSMPYFLVPINCLNAIVDWKSQNPMSIIDWETVEYVQDFENKQADKPWEHKPWLGKPDDYFKDKFITDPFDGSRKLWSVGITKEYRPLDPVPPNTAPRKGARKNNSNIMEYSCSLWAKARAKRTFDEEQPVIEATYISLRRNLLDEFDGGELETSKKCFIILEPLKVSPLPTTVVAMAYLLPAIIHRVESYLIALEATDLLHLDIRPDLALEAVTKDSDNSGEHGEEQTNFQRGMGNNYERLEFLGDCFLKMGTSISLYGLNPDSDEFRYHVDRMCLICNKNLFNTALKLELYKYIRSAAFNRRAWYPEGPELLRGKTATAPNTHKLGDKSVADVCEAMIGAALLSHHESKSMDNAVRAVTEVVNSDNHNAVVWSDYYKLYEKPKWQTATATAAQIDMARQVEMKHPYHFKHPRLLRSAFIHPAYLFIYEQIPCYQRLEFLGDSLLDMACVNFLFHNHPTKDPQWLTEHKMAIVSNQFLGALCVKLGFHKHLLTLDSQVQKMIADYSSDINEALIQAKTDAKRVGKVEDDYARDYWIAVRQPPKCLPDIVEAFIGAIFVDSEYDYGEVEKFFEMHIRWYFEDMGIYDTYANKHPTTFLTNFLQKNMGCEDWAPVSKEVPGEDGRKNVVVCGVIIHNKVVSTATAESMRYARVGAARNALRKLEGMSVREFRDEYGCSCEGDVVDEEGNIEFVEREDGMEGIGMGY

SEQ ID NO: 23 - DNA, 4872 bp, Botrytis cinerea
gactgcctgtaccgacattctcagcatttcttatcgaaattgagaatgtcgtacaaaggtaaaaaaaaaaaaatgacaaatgaaagtccataaattcccaaaatggacaactattcgcggaaacaaaagaccccggtcgcaaaaacaaacgcctcatcttaaccacgactctctatctctcaaacccacatcgctcttccacaattcctcattcccagttagggtttttagtctaacgaatcattttcgctctccggcttctttcgaggacatggaatacacttcggaacctgacactgacccggatacacgcggtagccttatcgatggtcgagatgggattgaaggggatcttattgctttgacgtctggggaacgacttaatgagactgtagaggatttatgtagtgactcatcaggattgattgttgagaatgaagatgatgataacagcgcaggggagaagggagagattgtgatagtaacaccaagaacataccaactggaaatgttggaagagagtttgaaaaggaatgtcatcgttgcgatggatacaggaagtggcaagacacatgtggccgttctccgaatactagcggaacttgagcggatgaagcctggcaagataatatggtttcttgcgcctaccgttgcgctctgtgctcagcatcacgaatatctccagctgaatattccctctgttttgatcaaaatgcttattggtgctgatggtgtggatcgatggacagagcagagacagtgggatacggtcttgaaggatgtcaaggtagtcgtatcttcctatcaagttcttctagatgcccttacacacggattcgtacgcatggggcgtctgtccttgatcatttttgatgaagcacataattgtgtaaataaagcgccaggggctaaaattatgaaatctttctatcatccgtataaatcgatattcccacttccccacattctgggcctctcggccagccctgtcatgagatccagtccacaatctttaagtgatatcgaggagactttggatgccatttgctgcacgccaaaaatacatcgagcagatcttcgccttcgagtaaagctaccacttctatctattatctactataccccagagtcaaatatcatcgtgacgaaaactgtggcgagcctgagaaagattgtgcaaagtctcaacattttcgaagacccctacgttttgacactaaaaaggagtgatagcgaaaaaagtcaacgtgagctggcgaaagtactcaagagttttaagacatatagtcaaacccaattaaagtcaatcgacaaaactagcaacgagattattcttgtagagctaggcccatgggctgcagattactatatctcaacagtggtgacgagatacttgaaggcaatgtcggcaaaggacactttcattgttgaagattcaccagctgccgagaagctatatattgccaaggctctcagacaagtcgaaatctctccttcaactctctcagatacaggcaaaatttctaacaaggttgaaaagctactggggataattgcgcaacagaagcctcccttttccgctattatatttgtccaagaaagagccacggtgtctgtgctagcccatctattatcgcatcatccattgacaaaggatcgttttaagattggaaccatggttggcacatccttaaatggcaagcgtacagaccaaataggagagcttgtcgatgttaatcaacaaaaagacactttgtcaagtttcaagcgtggaaaaattgatatccttatagctacaaatgtattggaagagggaattgatgttcctgcctgtaatctagtgatctgctttagtaaaccagcaaacctcaaatctttcgtacaaagacgagggcgagcaagacagcaagattctaagctgattcttcttgatgcttcaggtgataaagcgacaaattggcatgagcttgaaagaaaaatgcgagaggagtacggaaaggaaatgcgagaattgcaacacatctacgaaattgagacagctgatgaacagtcggaagatgatagggtcttgcgaatagaaagcactggggctcaattagaccttgacagtgctttaccacatctctatcatttctgttcagtcttaacaacaaaagattttgttgacctcaggccagacttcgtctactcctccgaactgggatcggaatatgttcgagcaaaggtcatcctgcctggatcggtttctaaacccctgcgagtccatgaaagccgcggatcgtggttgagcgagaggtcggctgcaaaagatgcagcgtttgaggcgtattccgcattatacagggggggcttagtgaatgataacctactgcccctgatggtgcacgacaaagtcatcgatgagttgacttcaaagcccgtggatactcgcgcgtctcttctggaggtgaaggaaagattaaatccatggattgacattgctagagcatggaaagaggcagaacaccatgctggaattgttcgcacatcggtaatgatcttcaatgggatgaagctggaactctgtcttccaattgatccaccggcaatacccccattaaagctttattgggatgctgacaccgagttctttgttgactttacaaacgatatcgagatcggcaccagcgagaatatgttggcacaggcgttgaacgataccaatctactattatcagatcgtggtcgtaaagttcacatccagtcacgtcgaacagttgtgcaatttatcttgcttcaagattcgggctcgctcagttcagattgttttccggttgaccccaacggtaatattaaaagtacaggttttatcagagaagtcggtaaactagaatcgccctacatctttgaaaaatggttgcccagtaagccaccgatcaatcaagtcaagaacccttatccagactttccagatgcaccagaagacgtcccatatctagctgtggttaaagtaagtcgccgtgcagactttttgcacaaggtacagaacgaaaaaccctcgtcattcactaaacaattctcgtctgttctacctgcctcgacatgtgtacaggatgtaatgcccgcacagttgtctcggttcggcatgatgattccttccatcacacaccacattgaggtgcaactcgttgtagaccgactatccaggaccatcctcaaggatctcgaaattagtgaccagagtcttattcagaccgccatcacacatgccagttattcgttagactcgaattatcagcgtctcgaatttctgggcgactcaattctcaaattgtgtacatcggtacaattggtggcagagcatctagattggcacgaaggatatttgtcggctatgaaggatcgtatcgtgtccaattcacggtcatcaagagcggcggctgaagtcggtttggatgagtatataatgaccaagaaattcacaggtgcaaaatggcgaccaatgtacgtggatgatctggtcgtcacagaacaaaaaacaagagaaatgtcctccaaaattctttccgacgttgtggaagcactcatcggcgcatgtatggtcgatggcggcattcctaaagcccttaaatctctacaactattcttcccagaactcaactggctccccttagaaactcgacaaatgacactctaccaacaagctgctaatgatctccatctacctatagctctccggcccgtcgagcaaatcctcgcatataccttcaccaaaaaatctctcctcgtcgaagccatgacgcacccctcttacaccagcggcacgcaatccctcgagcgactcgagttcctcggcgattccattctcgacaacatcatcgtcacagccatgtggtcgcactcgacgccgctctcccacttccacatgcatctcctgcgctctgcgctcgtcaacgccgatttcctcgcctttctctgcatggaaatgagcatcgaccaaaacgtcaccaatctgaccgaaggaaaaaaccatcgcatccacgaaacccactcgcgacgccgcgtttccctcgtcagttttctccgtcactcaagcgttcgtctctctatctatcaaaaagaagcgctttctcgccatgcagaattgcgcgatcagatcctcgaggcaatatacaccggtgatacattcccctgggctctattatcccgattggacgcgcggaaatttttctccgatatgattgagagtttgctgggcgcggtatggattgatagcggctcgatggaagtgtgcacgcagctgatcgaaagaatgggcgtcctgagatacatgcgacggattttgaaagatggcgtgcgcatcatgcatccgaaggaggaactgggcatcgtggccgattctgaaaacgtcaggtacgttttgcggcgggagaagatgggtggggatgctaccgaggtaaatgcggacgcggatgaagaggtacgcacggagtaccggtgcacagtatttgtgggcggggaggaaattgtagaggtgaggggtggagcgaggaaagaggagattcaggcaagggctgcggagcaggcggtgcggattttgaaggcgaggggtcatgagaagaggaatgggggtgcgggggaggggaaaaagagaaaatcgctggatgaatagagagcatttgtaggggaaggaggaaaaattgaggaggaggataagatgaattttgataaatttatttcctaacatcaggtcacaatctatgaattacatttgatagtattacgtatgccggtctgtacacaacacaaccatatagtaaggtatcaatcaaatgcgatggatagtcatttcaatttcttagtgaataattacaacgaaccagtaaaatagca

SEQ ID NO: 24 - protein, 1523 aa, Botrytis cinerea
MKVHKFPKWTTIRGNKRPRSQKQTPHLNHDSLSLKPTSLFHNSSFPVRVFSLTNHFRSPASFEDMEYTSEPDTDPDTRGSLIDGRDGIEGDLIALTSGERLNETVEDLCSDSSGLIVENEDDDNSAGEKGEIVIVTPRTYQLEMLEESLKRNVIVAMDTGSGKTHVAVLRILAELERMKPGKIIWFLAPTVALCAQHHEYLQLNIPSVLIKMLIGADGVDRWTEQRQWDTVLKDVKVVVSSYQVLLDALTHGFVRMGRLSLIIFDEAHNCVNKAPGAKIMKSFYHPYKSIFPLPHILGLSASPVMRSSPQSLSDIEETLDAICCTPKIHRADLRLRVKLPLLSIIYYTPESNIIVTKTVASLRKIVQSLNIFEDPYVLTLKRSDSEKSQRELAKVLKSFKTYSQTQLKSIDKTSNEIILVELGPWAADYYISTVVTRYLKAMSAKDTFIVEDSPAAEKLYIAKALRQVEISPSTLSDTGKISNKVEKLLGIIAQQKPPFSAIIFVQERATVSVLAHLLSHHPLTKDRFKIGTMVGTSLNGKRTDQIGELVDVNQQKDTLSSFKRGKIDILIATNVLEEGIDVPACNLVICFSKPANLKSFVQRRGRARQQDSKLILLDASGDKATNWHELERKMREEYGKEMRELQHIYEIETADEQSEDDRVLRIESTGAQLDLDSALPHLYHFCSVLTTKDFVDLRPDFVYSSELGSEYVRAKVILPGSVSKPLRVHESRGSWLSERSAAKDAAFEAYSALYRGGLVNDNLLPLMVHDKVIDELTSKPVDTRASLLEVKERLNPWIDIARAWKEAEHHAGIVRTSVMIFNGMKLELCLPIDPPAIPPLKLYWDADTEFFVDFTNDIEIGTSENMLAQALNDTNLLLSDRGRKVHIQSRRTVVQFILLQDSGSLSSDCFPVDPNGNIKSTGFIREVGKLESPYIFEKWLPSKPPINQVKNPYPDFPDAPEDVPYLAVVKVSRRADFLHKVQNEKPSSFTKQFSSVLPASTCVQDVMPAQLSRFGMMIPSITHHIEVQLVVDRLSRTILKDLEISDQSLIQTAITHASYSLDSNYQRLEFLGDSILKLCTSVQLVAEHLDWHEGYLSAMKDRIVSNSRSSRAAAEVGLDEYIMTKKFTGAKWRPMYVDDLVVTEQKTREMSSKILSDVVEALIGACMVDGGIPKALKSLQLFFPELNWLPLETRQMTLYQQAANDLHLPIALRPVEQILAYTFTKKSLLVEAMTHPSYTSGTQSLERLEFLGDSILDNIIVTAMWSHSTPLSHFHMHLLRSALVNADFLAFLCMEMSIDQNVTNLTEGKNHRIHETHSRRRVSLVSFLRHSSVRLSIYQKEALSRHAELRDQILEAIYTGDTFPWALLSRLDARKFFSDMIESLLGAVWIDSGSMEVCTQLIERMGVLRYMRRILKDGVRIMHPKEELGIVADSENVRYVLRREKMGGDATEVNADADEEVRTEYRCTVFVGGEEIVEVRGGARKEEIQARAAEQAVRILKARGHEKRNGGAGEGKKRKSLDE